(19) United States
(12) Patent Application Publication
(10) Pub. No.: US 2006/0211736 A1
(43) Pub. Date: Sep. 21, 2006

(54) GLUTARIMIDE-CONTAINING POLYKETIDE ANALOGS AND METHODS THEREOF

(75) Inventor: Ben Shen, Verona, WI (US)

Correspondence Address: GODFREY & KAHN, S.C., 780 N. WATER STREET, MILWAUKEE, WI, 53202, US

(73) Assignee: Wisconsin Alumni Research Foundation, Madison, WI (US)

(21) Appl. No.: 11/275,556

(22) Filed: Jan. 13, 2006

Related U.S. Application Data
(60) Provisional application No. 60/593,434, filed on Jan. 13, 2005.

(51) Int. Cl. A61K 31/452; A61K 31/445; C07H 21/04; C12P 17/00; C07D 405/02; C12N 1/21; C07D 211/76

(52) U.S. Cl. 514/326; 514/327; 435/117; 435/252.35; 536/23.2; 546/207; 546/243

(57) ABSTRACT

The present invention provides library of glutarimide-containing polyketide analogs, such as analogs of migrastatin, iso-migrastatin, dorrigocin A and B, epi-dorrigocin, NK30424 A and B and lactimidomycin, methods of synthesizing and using these analogs and further methods of creating a combinatorial library of these compounds through chemical modifications.

### RELATED APPLICATION

The present application seeks priority from U.S. Provisional Application No. 60/593,434 filed on Jan. 13, 2005 which is incorporated herein by reference for all purposes.

### STATEMENT AS TO RIGHTS TO INVENTIONS MADE UNDER FEDERALLY SPONSORED RESEARCH AND DEVELOPMENT

This work was supported in part by a grant from the National Institutes of Health grants CA106150 and A1051689. The Government of the United States of America may have certain rights in this invention.

### FIELD OF INVENTION

The present invention generally relates to glutarimide-containing polyketide compounds and related gene clusters and specifically to migrastatin and dorrigocin analogs, related gene clusters and methods of synthesis and uses of these compounds and gene clusters.

### BACKGROUND:

Migrastatin (MGS) (FIG. 1) was first isolated from a Streptomyces species in 2000 and revealed to be a 14-membered macrolide with a glutarimide side chain. Nakae, K.; Yoshimoto, Y.; Sawa, T.; Homma, Y.; Hamada, M.; Takeuchi, T.; Imoto, M. “Migrastatin, a new inhibitor of tumor cell migration from Streptomyces sp. MK929-43F1: taxonomy, fermentation, isolation and biological activities” J. Antibiot. 2000, 53, 1130-1136; Nakae, K.; Yoshimoto, Y.; Ueda, M.; Sawa, T.; Takahashi, Y.; Naganawa, H.; Takeuchi, T.; Imoto, M. “Migrastatin, a novel 14-membered lactone from from Streptomyces sp. MK929-43F1” J. Antibiot. 2000, 53, 1228-1230. Its relative and absolute stereochemistry was subsequently determined by X-ray crystallography analysis of a derivative of MGS. Nakamura, H.; Takahashi, Y.; Naganawa, H.; Nakae, K.; Imoto, M.; Shiro, M.; Matsumura, K.; Watanabe, H.; Kitahara, T. “Absolute configuration of migrastatin, a novel 14-membered ring macrolide” J. Antibiot. 2002, 55, 442-444. MGS displays a remarkable inhibitory effect on human tumor cell migration (IC₅₀=1.0 pg/ml). Nakae, K.; Yoshimoto, Y.; Ueda, M.; Sawa, T.; Takahashi, Y.; Naganawa, H.; Takeuchi, T.; Imoto, M. “Migrastatin, a novel 14-membered lactone from from Streptomyces sp. MK929-43F1” J. Antibiot. 2000, 53, 1228-1230. Takwmoto, Y.; Nakae, K.; Kawatani, N.; Takahashi, Y.; Naganawa, H.; Imoto, M. “Migrastatin, a novel 14-membered ring macrolide, inhibits anchorage-independent growth of human small cell lung carcinoma Ms-1 cells” J. Anbtibiot. 2001, 54, 1104-1107.

Since development of metastatic lesions remains the predominant cause of death for most cancer patients and cell migration is essential for invasion of the extracellular matrix and for cell dissemination during metastasis, inhibition of tumor cell migration represents a potential therapeutic approach for the treatment of tumor metastasis. MGS could therefore be a valuable tool for elucidating the signal transduction pathways involved in cell migration or serve as a lead for the development of therapeutic agents for treating tumor metastasis. Gaul, C.; Danishefsky, S. J. “Synthesis of the macrolide core of migrastatin” Tetrahedron Lett. 2002, 43, 9039-9042; Njardarson, J. T.; Gaul, C.; Shan, D.; Huang, X.-Y.; Danishefsky, S. J. “Discovery of potent cell migration inhibitors through total synthesis: lessons from structure-activity studies of (+)-migrastatin” J. Am. Chem. Soc. 2004, 126, 1038-1040.

Dorrigocins (DGNs) were first isolated from Streptomyces platensis in 1994, and their structures were established to be glutarimide-containing linear polyketides with DGN A and B being regio-isomers. Karwowski, J. P.; Jackson, M.; Sunga, G.; Sheldon, P.; Poddig, J. B.; Kohl, W. L.; Kadam, S. “Dorrigocins: novel antifungal antibiotics that change the morphology of ras-transformed NIH/3T3 cells to that of normal cells: Taxonomy of the producing organism, fermentation and biological activity” J. Antibiot. 1994, 47, 862-869. Hochlowski, J. E.; Whittern, D. N.; Hill, P.; McAlpine, J. B. “Dorrigocins: novel antifungal antibiotics that change the morphology of ras-transformed NIH/3T3 cells to that of normal cells: Isolation and elucidation of structures” J. Antibiot. 1994, 47, 870-874. The stereochemistry of DGNs remains to be established. The DGNs were described as the first natural product inhibitors of the carboxyl methyltransferase involved in the processing of Ras-related proteins, affecting signal transduction in eukaryotic cells (e.g. 0.4 ∝g/ml DGN A caused 74% reduction of the number of foci produced by K-ras transformed NIH/3T3 mouse fibroblasts). Kadam, S.; McAlpine, J. B. “Dorrigocins: novel antifungal antibiotics that change the morphology of ras-transformed NIH/3T3 cells to that of normal cells: Biological properties and mechanism of action” J. Antibiot. 1994, 47, 875-880. Winter-Vann, A. M.; Kamen, B. A.; Bergo, M. O.; Young, S. G.; Melnyk, S.; James, J.; Casey, P. J. “Targeting Ras signaling through inhibition of carboxyl methylation: an unexpected property of methotrexate” Proc. Natl. Acad. Sci. 2003, 100, 6529-6534. Since Ras oncoproteins have been implicated in the pathogenesis of many types of cancers, inhibition of the latter enzyme represents a potential therapeutic approach for cancer treatment. Downward, J. “Targeting Ras signaling pathways in cancer therapy” Nat. Rev. Cancer 2003, 3, 11-22; Dancey, J. E. “Agents targeting Ras signaling pathways” Curr. Pharm. Des. 2002, 8, 2259-2267. DGNs, therefore, provide a valuable tool for further studies on the role of carboxyl methylation in the processing of Ras-related proteins and cellular signal transduction. The ultimate utility of DGNs will depend on the generation of DGN analogs with improved selectivity for carboxyl methyltransferases required for the processing of Ras-related proteins. Dancey, J. E. “Agents targeting Ras signaling pathways” Curr. Pharm. Des. 2002, 8, 2259-2267.

DGN A can be viewed as an acyclic isomer of MGS, leading to the hypothesis that DGNs and MGS might be biosynthesized by the same pathway. Careful reinvestigation of all metabolites produced by S. platensis, which was previously only known as a DGN producer, indeed confirmed the production of MGS as well as a new MGS analog, iso-MGS. Woo, E. J.; Starks, C. M.; Carney, J. R.; Arslanian, R.; Cadapan, L.; Zavala, S.; Licari, P. “Migrastatin and a new compound, isomigrastatin, from Streptomyces platensis” J. Antibiot. 2002, 55, 141-146. Consistent with the structural relationship between DGN A and MGS, DGN B is an acyclic isomer of iso-MGS. Although the precise mechanism of how these regio-isomers are biosynthesized remains to be established, co-isolation of these metabolites supports the hypothesis that MGS and DGN share the same biosynthetic pathway.

Lactimidomycin, LTM was isolated from Streptomyces amphibiosporus ATCC53964 in 1992. Sugawara, K.; Nishiyama, Y.; Toda, S.; Komiyama, N.; Hatori, M.; Moriyama, T.; Sawada, Y, Kamei, H.; Konishi, M.; Oki, T. “Lactimidomycin, a new glutarimide group antibiotic: production, isolation, structure and biological activity” J. Antibiot. 1992, 45, 1433-1441. Its structure was established to be a 12-membered macrolide with a glutarimide side chain, and its polyketide biosynthetic origin was confirmed by feeding experiments with ¹³C-labelled precursors. The absolute stereochemistry of LTM remains to be determined. LTM exhibits potent cytoxicity and in vivo antitumor activity. For example, LTM showed potency ca. 20˜600 times superior to MTM C in terms of IC₅₀ value with various tumor cell lines in vitro and prolonged the survival time of mice transplanted with experimental tumors with efficacy comparable to or better than MTM C in vivo (e.g. 1.4 ng/ml for LTM in comparison to 800 ng/ml for MTM C under the same assay conditions with HCT-116 cells). In addition, it showed antifungal activity and inhibited both DNA and protein syntheses. Sugawara, K.; Nishiyama, Y.; Toda, S.; Komiyama, N.; Hatori, M.; Moriyama, T.; Sawada, Y.; Kamei, H.; Konishi, M.; Oki, T. J. Antibiot. 1992, 45, 1433-1441.

Aimed at preparing structural analogs with improved biological profiles, synthetic strategies towards the MGS scaffold have appeared, leading to the synthesis of the macrolide core as well as a small library of MGS analogs. Gaul, C.; Danishefsky, S. J. “Synthesis of the macrolide core of migrastatin” Tetrahedron Lett. 2002, 43, 9039-9042. Njardarson, J. T.; Gaul, C.; Shan, D.; Huang, X.-Y.; Danishefsky, S. J. “Discovery of potent cell migration inhibitors through total synthesis: lessons from structure-activity studies of (+)-migrastatin” J. Am. Chem. Soc. 2004, 126, 1038-1040. Strikingly, analogs with IC₅₀ in the cell migration assay at low nM range, which was reduced by ca. 3 orders of magnitude relative to MGS, have been synthesized, supporting the strategy to discover novel cell migration inhibitors building on the MGS molecular scaffold. No synthesis for DGNs and LTM is known.

Two additional natural products structurally related to MGS and DGN are NK30424 A and B, which were isolated from Streptomyces sp. NA30424 by Takayasu and co-workers in 2001. Takayasu, Y.; Tsuchiya, K.; Aoyama, T.; Sukenaga, Y. “NK30424A and B, novel inhibitors of lipopolysaccharide-induced tumor necrosis factor alpha production, produced by Streptomyces sp. NA30424” J. Antibiot. 2001, 54, 111-1115. Spectroscopic analyses revealed that NK30424 A and B were new members of the glutarimide-containing macrolide family of antibiotics. NK30424 A and B are stereoisomer, and the stereochemistry for both compounds remains to be established. Interestingly, NK30424 share the same glutarimide side chain as LTM and the same 12-membered macrolide core as iso-MGS upon conceptual Michael addition of a cysteine to the C-3 of the marolide core. NK30424 A and B inhibit lipopolysaccharide (LPS)-induced tumor necrosis factor alpha (TNF-α) production by suppression of the NF-κB signaling pathway. The NF-κB signaling pathway is thought to be the target for anti-inflammatory agents because it activates various gene expressions involved in inflammation. Increasing evidence also supports that NF-κB might play a role in regulating cell growth, oncogenesis, and protection from cell death. Cordie, S. R.; Donald, R.; Read, M. A.; Hawiger, J. “Lopopolysaccharide induces phosphorylation of MAD3 and activation of c-Rel and related NK-κB proteins in human monocytic THF-1” J. Biol Chem. 1988, 268, 11803-11810; Karin, M. “How NF-κB is activated: the role of the IκB kinase (IKK) complex” Oncogene 1999, 18, 6867-6874; Rayet, B; Gelinas, C. “Aberrant rel/nfkb genes and activity in human cancer” Oncogene 1999, 18, 6938-6947. Therefore, inhibitors of LPS-induced TNF-α production could have the potential for the treatment of NF-κB signaling pathway-mediated pathophysiological diseases.

Taken together, MGS, DGN, LTM, and NK30424 A and B encompass a broad spectrum of biological activities, supporting the wisdom of making structural analogs based on these molecular scaffolds for new drug discovery and development. Development of strategies and methods to manufacture these analogs, particularly of those that are unavailable or extremely difficult to prepare by chemical synthesis, therefore, is an important research goal to develop these leads into clinically useful drugs.

### SUMMARY OF THE INVENTION

The present invention generally provides a biocombinatorial library of glutarimide compounds and methods related to synthesis and use of glutarimide-containing polyketide analogs. In this invention the glutarimide-containing polyketide analogs created by the library generally refer to iso-migrastatin analogs, migrastatin analogs, lactimidomycin analogs, dorrigocin A and B analogs, epi-dorrigocin analogs, NK30424 A and B analogs, and the like.

In one preferred embodiment, the present invention provides a glutarimide-containing polyketide analog selected from the group consisting of:

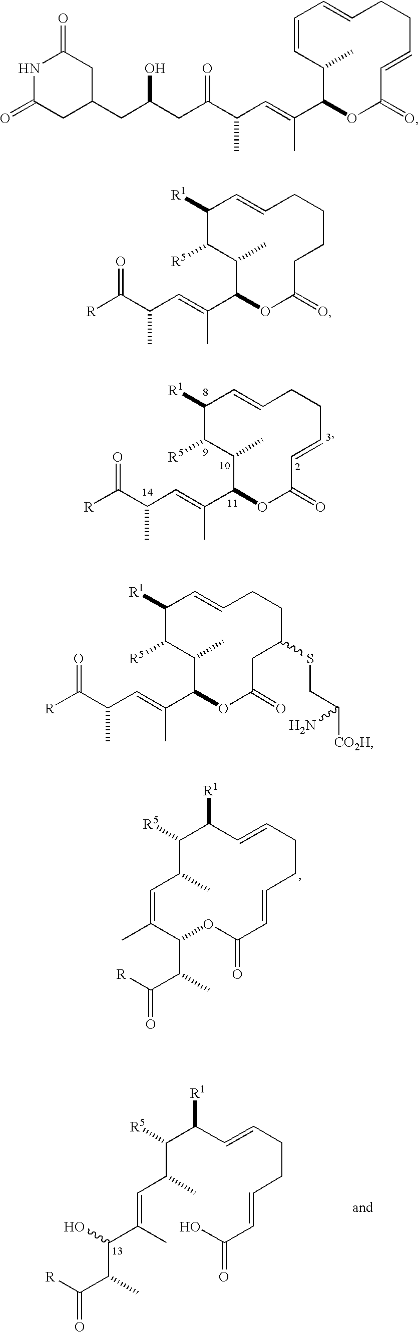

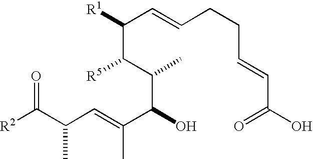

wherein R is selected from the group consisting of:

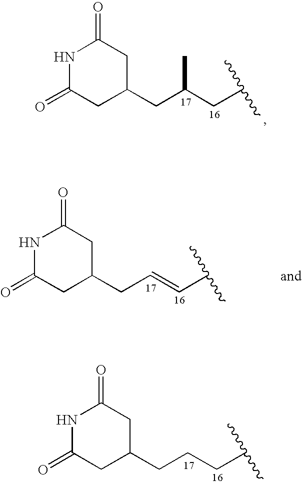

wherein R¹ is selected from the group consisting of H, OH, OCH₃; and wherein R⁵ is selected from the group consisting of H, OH, OCH₃, with the provisio that in compound

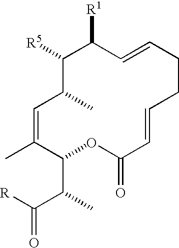

R is not

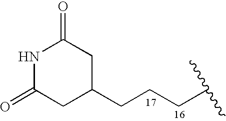

when R¹ is OCH₃ and R⁵ is OH.

Another embodiment of the present invention provides a method of synthesizing a glutarimide-containing polyketide analog. One such method comprises the steps of: (a) fermenting a wild type strain of Streptomyces amphibiosporus or Streptomyces platensis in a culture medium under conditions whereby said wild type strain expresses said glutarimide-containing polyketide analog; and (b) isolating and purifying at least one glutarimide-containing polyketide analog. Another such method comprises the steps of: (a) fermenting a mutant strain of Streptomyces amphibiosporus or Streptomyces platensis in a culture medium under conditions whereby said mutant strain expresses said glutarimide-containing polyketide analog; and (b) isolating and purifying at least one glutarimide-containing polyketide analog. In this method, the mutant strain of Streptomyces amphibiosporus or Streptomyces platensis comprises a nucleic acid selected from the group consisting of SEQ ID. 1, SEQ ID. 2, SEQ ID. 3, SEQ ID. 4, and combinations thereof.

Yet another method for synthesizing a glutarimide-containing polyketide analog comprises the steps of (a) fermenting a recombinantly modified iso-migrastatin or lactimidomycin gene cluster under conditions whereby said gene cluster expresses said glutarimide-containing polyketide analog; and (b) isolating at least one glutarimide-containing polyketide analog. In this method, the gene cluster is present in a bacterium selected from the group consisting Streptomyces amphibiosporus and Streptomyces platensis.

The invention further teaches methods for chemically modifying the glutarimide-containing polyketide analog thus creating a library of polyketide analogs. In a preferred embodiment, the present invention provides a method of chemically modifying a glutarimide-containing polyketide analog said method comprising: (I) obtaining an isolated glutarimide-containing polyketide analog, as described above; (II) conducting at least one of the modification to said glutarimide-containing polyketide analog to result in a chemically modified glutarimide-containing polyketide analog, wherein the modification is selected from the group consisting of: (a) a water mediated ring opening of the glutarimide-containing polyketide analog; (b) 1,4-addition of the glutarimide-containing polyketide analog by a sulfur-containing nucleophile, such as by Michael's addition or other methods known to one of ordinary skill in the art; (c) regioselective 1,4-reduction of the glutarimide-containing polyketide analog, such as with Stryker's reagent or other reducing agents known to one of ordinary skill in the art; and (d) N-acylation of the glutarimide moiety of the glutarimide-containing polyketide analog; and (III) isolating and purifying at least one chemically modified glutarimide-containing polyketide analog.

Yet another embodiment of the invention provides a glutarimide-containing polyketide analog produced by the methods of described above.

Another embodiment of the present invention provides a pharmaceutical composition comprising a glutarimide-containing polyketide analog as described above; or its pharmaceutically-acceptable salt; and a pharmaceutically-acceptable carrier.

The present invention also provides an isolated, purified or enriched nucleic acid comprising a sequence selected from the group consisting of SEQ ID. Nos. 1, 2, 3 and 4; the sequences complimentary to SEQ ID. Nos. 1, 2, 3 and 4 and fragments comprising at least 10 consecutive nucleotides of the sequences complementary to SEQ ID. Nos. 1, 2, 3 and 4. In another embodiment, the present invention provides an isolated, purified or enriched nucleic acid capable of hybridizing to the nucleic acid comprising a sequence selected from the group consisting of SEQ ID. Nos. 1, 2, 3 and 4 under conditions of high stringency.

Further objects, features and advantages of the invention will be apparent from the following detailed description, drawings and appended claims.

### BRIEF DESCRIPTION OF FIGURES

FIG. 1. Glutarimide-containing polyketide natural/synthetic products.

FIG. 2. (A) Structures of iso-migrastatin (4) and its shunt metabolites migrastatin (1 ), dorrigocin A (2), B (3), and 13-epi-dorrigocin A (5) and HPLC profiles of (B) EtOAc extract of fermentation broth in the absence of resin, (C) MeOH eluent of XAD-16 resin harvested from fermentation broth, (D) purified 4, and (E) incubation of 4 [1 mM solution of 4 in H₂O-DMSO (9:1)] at 37° C. for 2 hrs. (♦) and (∇), metabolites whose structures have not been fully established.

FIG. 3. Proposed mechanism for H₂O-mediated rearrangement of 4 to 1, 2, 3, and 5 as supported by the incorporation of 180 from H₂¹⁸O.

FIG. 4. Structures of selected glutarimide-containing polyketide natural products and isolation of iso-migrastatin (110) and its congeners (112-119) and their conversion into a glutarimide polyketide library featuring the dorrigocin (102, 103, 111 ) lactimidomycin (104), migrastatin (101), and NK30442 A and B (105, 106) scaffolds via H₂O-mediated rearrangement or cysteine 1,4-addition, respectively.

FIG. 5. Post-PKS processing steps in the biosynthesis of iso-migrastatin (10) from the nascent polyketide intermediate (119) in S. platensis NRRL18993 (SAM, S-adenosylmethionine and NH₃, an unspecified amino donor). DH, dehydratase; ER, enoyl reductase; MT, methyltransferase; OH, hydroxylase.

FIG. 6. HPLC-UV (PDA)-ESI-MS analyses of resin extracts from fermentations of strain S. platensis with 110 and its eight congeners, 112-119, shown as distinct peak. (A) HPLC profile with UV detection at 205 nm. (B) Total ion chromatograms (TIC) from LC-ESI-MS (+). (C) TIC from LC-ESI-MS (−). (D) LC-ESI-MS (−) selected for amu=490, corresponding to 116. Peak identification: 110 (isomigrastatin), 112 (16,17-didehydro-isomigrastatin), 113 (17-hydroxy-isomigrastatin), 114 (8-desmethyl-isomigrastatin), 115 (16,17-didehydro-8-desmethyl-isomigrastatin), 116 (17-hydroxy-8-desmethyl-isomigrastatin), 117 (8-desmethoxy-isomigrastatin), 118 (16, 17-didehydro-8-desmethoxy-isomigrastatin), 119 (17-hydroxy-8-desmethoxy-isomigrastatin).

FIG. 7. Selected COSY (bold) and gHMBC (arrow) correlations observed in compounds 112-115 and 117-119.

FIG. 8. HPLC chromatograms of purified 112-114 and 117-119, together with their corresponding H₂O-mediated rearrangement products. (A), products of 110; (B), pure 112; (C), products of 112; (D), pure 113; (E), products of 113; (F), pure 114; (G), products of 114; (H), pure 117; (I), products of 117; (j), pure 118; (K), products of 118; (L), pure 119; (M), products of 119. Peak identification: see table 6.

FIG. 9. Selected COSY (bold) and gHMBC (arrow) correlations observed in compounds 120-125.

FIG. 10. HPLC chromatograms of products resulted from L-cysteine 1,4-Michael addition of 110 (A and F), 113 (B and G), 114 (C), 117 (D) and 119 (E). While panels A-G were developed with a linear gradient of 15% CH₃CN to 80% CH₃CN in H₂O containing 0.1% HOAc over 20 min, panels F and G were developed with a linear gradient of 20% MeOH to 80% MeOH in H₂O over 20 min.

FIG. 11. 12-membered macrolactones purified from wild-type and recombinant mutant strains.

FIG. 12. H₂O-mediated rearrangement of 203 into 13-epi-dorrigocin A (203a), dorrigocin B (203b), dorrigocin A (203c), and migrastatin (203d).

FIG. 13. 1,4-Michael addition of cysteine to the C2/C3 double bond.

FIG. 14. 1,4-reduction of C2/C3 double bond by Stryker's reagent.

FIG. 15. N-acylation of the glutarimide moiety.

FIG. 16. 14-membered migrastatin derivatives generated via H₂O-mediated rearrangements.

FIG. 17. 13-epi-dorrigocin A derivatives generated via H₂O-mediated rearrangements.

FIG. 18. Dorrigocin A derivatives generated via H₂O-mediated rearrangements.

FIG. 19. Dorrigocin B derivatives generated via H₂O-mediated rearrangements.

### DETAILED DISCUSSION OF PREFERRED EMBODIMENTS

### I. GENERAL DESCRIPTION OF THE INVENTION

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

### DEFINITIONS

As defined herein, the term “isomer” includes, but is not limited to optical isomers and analogs, structural isomers and analogs, conformational isomers and analogs, and the like. In one embodiment, this invention encompasses the use of different optical isomers of a glutarimide-containing polyketide compounds. It will be appreciated by those skilled in the art that the compounds useful in the present invention may contain at least one chiral center. Accordingly, the compounds used in the methods of the present invention may exist in, and be isolated in, optically-active or racemic forms. Some compounds may also exhibit polymorphism.

It is to be understood that the present invention may encompass the use of any racemic, optically-active, polymorphic, or stereroisomeric form, or mixtures thereof, which form possesses properties useful in the treatment of tumor-related and other conditions described and claimed herein. In one embodiment, the polyketide compounds may include pure (R)-isomers. In another embodiment, the poyketide compounds may include pure (S)-isomers. Of course, these polyketide compounds may have more than one chiral center, in which case, in one embodiment, the polyketide compound may have chiral centers with R/S combinations. In another embodiment, the compounds may include a mixture of the (R) and the (S) isomers. In another embodiment, the compounds may include a racemic mixture comprising both (R) and (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes the use of pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also he prepared from the phenolic compounds by treatment with inorganic bases, for example, sodium hydroxide. Also, esters of the phenolic compounds can be made with aliphatic and aromatic carboxylic acids, for example, acetic acid and benzoic acid esters. As used herein, the term “pharmaceutically acceptable salt” refers to a compound formulated from a base compound which achieves substantially the same pharmaceutical effect as the base compound.

This invention further includes method utilizing derivatives of the polyketide compounds. The term “derivatives” includes but is not limited to ether derivatives, acid derivatives, amide derivatives, ester derivatives and the like. In addition, this invention further includes methods utilizing hydrates of the polyketide compounds. The term “hydrate” includes but is not limited to hemihydrate, monohydrate, dihydrate, trihydrate and the like.

This invention further includes methods of utilizing metabolites of the polyketide compounds. The term “metabolite” means any substance produced from another substance by metabolism or a metabolic process.

As defined herein, “contacting” means that the polyketide compound used in the present invention is introduced into a sample containing the receptor in a test tube, flask, tissue culture, chip, array, plate, microplate, capillary, or the like, and incubated at a temperature and time sufficient to permit binding of the compound to a receptor. Methods for contacting the samples with the compound or other specific binding components are known to those skilled in the art and may be selected depending on the type of assay protocol to be run. Incubation methods are also standard and are known to those skilled in the art.

In another embodiment, the term “contacting” means that the polyketide compound used in the present invention is introduced into a patient receiving treatment, and the compound is allowed to come in contact in vivo.

As used herein, the term “treating” includes preventative as well as disorder remittent treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing a patient, tissue, organ or cells in contact with a polyketide compound. As used herein, administration can be accomplished in vitro, i.e. in a test tube, or in vivo, i.e. in cells or tissues of living organisms, for example, humans. In certain embodiments, the present invention encompasses administering the compounds useful in the present invention to a patient or subject. A “patient” or “subject”, used equivalently herein, refers to a mammal, preferably a human, that either: (1) has a disorder remediable or treatable by administration of the polyketide compound or (2) is susceptible to a disorder that is preventable by administering the polyketide compound.

As used herein, “pharmaceutical composition” means therapeutically effective amounts of the polyketide compound together with suitable diluents, preservatives, solubilizers, emulsifiers, and adjuvants, collectively “pharmaceutically-acceptable carriers.” As used herein, the terms “effective amount” and “therapeutically effective amount” refer to the quantity of active therapeutic agent sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in one or more of the following: (a) the prevention of a disease; and (b) the reversal or stabilization of such disease. The optimum effective amounts can be readily determined by one of ordinary skill in the art using routine experimentation.

Pharmaceutical compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween (Polysorbate) 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

Also encompassed by the invention are methods of administering particulate compositions coated with polymers (e.g., poloxamers or poloxamines). Other embodiments of the compositions incorporate particulate forms protective coatings, protease inhibitors or permeation enhancers for various routes of administration, including topical, parenteral, pulmonary, nasal and oral. In one embodiment the pharmaceutical composition is administered parenterally, paracancerally, transmucosally, tansdermally, intramuscularly, intravenously, intradermally, subcutaneously, intraperitonealy, intraventricularly, intracranially and intratumorally.

Further, as used herein “pharmaceutically acceptable carriers” are well known to those skilled in the art and include, but are not limited to, 0.01-0.1 M and preferably 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions administerable according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Other embodiments of the compositions administered according to the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another method according to the invention, a pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, for example liver, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The pharmaceutical preparation can comprise the anti-tumor compound alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the polyketide compound can be administered to a patient by, for example, subcutaneous implantation of a pellet. In a further embodiment, a pellet provides for controlled release of the compound over a period of time. The preparation can also be administered by intravenous, intra-arterial, or intramuscular injection of a liquid preparation oral administration of a liquid or solid preparation, or by topical application. Administration can also be accomplished by use of a rectal suppository or a urethral suppository.

The pharmaceutical preparations administerable by the invention can be prepared by known dissolving, mixing, granulating, or tablet-forming processes. For oral administration, the polyketide compounds or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intra-arterial, or intramuscular injection), the polyketide compounds or their physiologically tolerated derivatives such as salts, esters, N-oxides, and the like are converted into a solution, suspension, or expulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts, which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the polyketide compounds or their physiologically tolerated derivatives such as salts, esters, N-oxides, and the like are prepared and applied as solutions, suspensions, or emulsions in a physiologically acceptable diluent with or without a pharmaceutical carrier.

In another method according to the invention, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein ibid., pp. 317-327; see generally ibid).

For use in medicine, the salts of the polyketide compound may be pharmaceutically acceptable salts. Other salts may, however, be useful in the preparation of the compounds according to the invention or of their pharmaceutically acceptable salts. Suitable pharmaceutically acceptable salts of the compounds include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid.

The terms “open reading frame”, and “ORF” refer to an open reading frame in the glutarimide-containing polyketide such as migrastatin biosynthesis gene cluster as isolated from Streptomyces platensis or Streptomyces amphibiosporus. The term also embraces the same open reading frames as present in other glutarimide-containing polyketide-synthesizing organisms (e.g. other strains and/or species of Streptomyces and the like). The term encompasses allelic variants and single nucleotide polymorphisms (SNPs). In certain instances the migrastatin ORF is used synonymously with the polypeptide encoded by the migrastatin ORF and may include conservative substitutions in that polypeptide. The particular usage will be clear from context.

The terms “isolated” “purified” or “biologically pure” refer to material which is substantially or essentially free from components which normally accompany it as found in its native state. With respect to nucleic acids and/or polypeptides the term can refer to nucleic acids or polypeptides that are no longer flanked by the sequences typically flanking them in nature.

Further, the term “isolated” means that the material is removed from its original environment, e.g. the natural environment if it is naturally occurring. For example, a naturally-occurring polynucleotide or polypeptide present in a living organism is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The term “purified” does not require absolute purity; rather, it is intended as a relative definition. Individual nucleic acids obtained from a library have been conventionally purified to electrophoretic homogeneity. The sequences obtained from these clones could not be obtained directly from a large insert library, such as a cosmid library, or from total organism DNA. The purified nucleic acids of the present invention have been purified from the remainder of the genomic DNA in the organism. However, the term “purified” also includes nucleic acids which have been purified from the remainder of the genomic DNA or from other sequences in a library or other environment by at least one order of magnitude, preferably two or three orders of magnitude, and more preferably four or five orders of magnitude.

“Recombinant” means that the nucleic acid is adjacent to “backbone” nucleic acid to which it is not adjacent in its natural environment. “Enriched” nucleic acids represent 5% or more of the number of nucleic acid inserts in a population of nucleic acid backbone molecules. “Backbone” molecules include nucleic acids such as expression vectors, self-replicating nucleic acids, viruses, integrating nucleic acids, and other vectors or nucleic acids used to maintain or manipulate a nucleic acid of interest. Preferably, the enriched nucleic acids represent 15% or more, more preferably 50% or more, and most preferably 90% or more, of the number of nucleic acid inserts in the population of recombinant backbone molecules.

The terms “polypeptide”, “peptide” and “protein” are used interchangeably herein to refer to a polymer of amino acid residues. The terms apply to amino acid polymers in which one or more amino acid residue is an artificial chemical analog of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49:1925) and references therein; Letsinger (1970) J. Org. Chem. 35:3800; Sprinzl et al. (1977) Eur. J. Biochem. 81: 579; Letsinger et al (1986) Nucl. Acids Res. 14: 3487; Sawai et al (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “heterologous” as it relates to nucleic acid sequences such as coding sequences and control sequences, denotes sequences that are not normally associated with a region of a recombinant construct, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

A “coding sequence” or a sequence which “encodes” a particular polypeptide (e.g. a PKS, etc.), is a nucleic acid sequence which is ultimately transcribed and/or translated into that polypeptide in vitro and/or in vivo when placed under the control of appropriate regulatory sequences. In certain embodiments, the boundaries of the coding sequence are determined by a start codon at the 5′ (amino) terminus and a translation stop codon at the 3′ (carboxy) terminus. A coding sequence can include, but is not limited to, cDNA from procaryotic or eucaryotic mRNA, genomic DNA sequences from procaryotic or eucaryotic DNA, and even synthetic DNA sequences. In preferred embodiments, a transcription termination sequence will usually be located 3′ to the coding sequence.

Expression “control sequences” refers collectively to promoter sequences, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Recombination” refers to the reassortment of sections of DNA or RNA sequences between two DNA or RNA molecules. “Homologous recombination” occurs between two DNA molecules which hybridize by virtue of homologous or complementary nucleotide sequences present in each DNA molecule.

The terms “stringent conditions” or “hybridization under stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 Overview of principles of hybridization and the strategy of nucleic acid probe assays, Elsevier, N.Y. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al. (1989) Molecular Cloning—A Laboratory Manual (2nd ed.) Vol. 1-3, Cold Spring Harbor Laboratory, Cold Spring Harbor Press, NY, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

Expression vectors are defined herein as nucleic acid sequences that are direct the transcription of cloned copies of genes/cDNAs and/or the translation of their mRNAs in an appropriate host. Such vectors can be used to express genes or cDNAs in a variety of hosts such as bacteria, bluegreen algae, plant cells, insect cells and animal cells. Expression vectors include, but are not limited to, cloning vectors, modified cloning vectors, specifically designed plasmids or viruses. Specifically designed vectors allow the shuttling of DNA between hosts, such as bacteria-yeast or bacteria-animal cells. An appropriately constructed expression vector preferably contains: an origin of replication for autonomous replication in a host cell, a selectable marker, optionally one or more restriction enzyme sites, optionally one or more constitutive or inducible promoters. In preferred embodiments, an expression vector is a replicable DNA construct in which a DNA sequence encoding a one or more PKS and/or NRPS domains and/or modules is operably linked to suitable control sequences capable of effecting the expression of the products of these synthase and/or synthetases in a suitable host. Control sequences include a transcriptional promoter, an optional operator sequence to control transcription and sequences which control the termination of transcription and translation, and so forth.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

“Single nucleotide polymorphism” or “SNPs are defined by their characteristic attributes. A central attribute of such a polymorphism is that it contains a polymorphic site, “X,” most preferably occupied by a single nucleotide, which is the site of the polymorphism's variation (Goelet and Knapp U.S. patent application Ser. No. 08/145,145). Methods of identifying SNPs are well known to those of skill in the art (see, e.g., U.S. Pat. No. 5,952,174).

The present invention generally provides a combinatorial library of glutarimide-containing polyketide compounds using basic scaffolds described here and methods related to synthesis and use of these analogs. In this invention the glutarimide-containing polyketide compounds and or analogs generally refer to migrastatin analogs, iso-migrastatin analogs, lactimidomycin analogs, dorrigocin A and B analogs, epi-dorrigocin analogs, NK30424 A and B analogs, and the like.

In one preferred embodiment, the present invention provides a glutarimide-containing polyketide analog selected from the group consisting of:

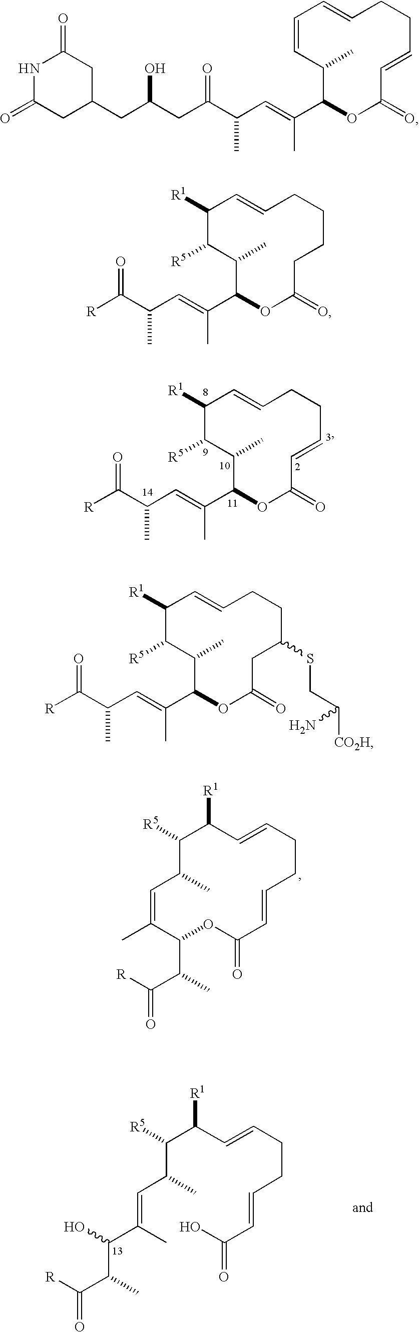

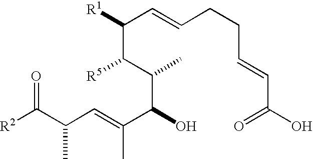

wherein R is selected from the group consisting of:

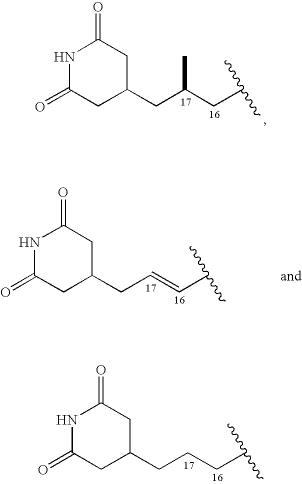

wherein R¹ is selected from the group consisting of H, OH, OCH₃; and wherein R⁵ is selected from the group consisting of H, OH, OCH₃, with the provisio that in compound

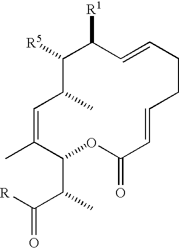

R is not

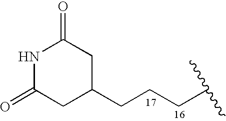

when R¹ is OCH₃ and R⁵ is OH.

Another embodiment of the present invention provides a method of synthesizing a glutarimide-containing polyketide analog. One such method comprises the steps of: (a) fermenting a wild type strain of Streptomyces amphibiosporus or Streptomyces platensis in a culture medium under conditions whereby said wild type strain expresses said glutarimide-containing polyketide analog; and (b) isolating and purifying at least one glutarimide-containing polyketide analog. Another such method comprises the steps of: (a) fermenting a mutant strain of Streptomyces amphibiosporus or Streptomyces platensis in a culture medium under conditions whereby said mutant strain expresses said glutarimide-containing polyketide analog; and (b) isolating and purifying at least one glutarimide-containing polyketide analog. In this method, the mutant strain of Streptomyces amphibiosporus or Streptomyces platensis comprises a nucleic acid sequence selected from the group consisting of SEQ ID. 1, SEQ ID. 2, SEQ ID. 3, SEQ ID. 4, and combinations thereof. Following is a list of sequences appended in the application:
(1) SEQ ID. 1: DNA sequence of the ItmK gene from S. amphibiosporus;
(2) SEQ ID. 2: DNA sequence of the mgsK gene from S. platensis;
(3) SEQ ID. 3: DNA sequence of the mgsJ gene from S. platensis
(4) SEQ ID. 4: DNA sequence of the mgsI gene from S. platensis

Yet another method for synthesizing a glutarimide-containing polyketide analog comprises the steps of (a) fermenting a recombinantly modified iso-migrastatin or lactimidomycin gene cluster under conditions whereby said gene cluster expresses said glutarimide-containing polyketide analog; and (b) isolating at least one glutarimide-containing polyketide analog. In this method, the gene cluster is present in a bacterium selected from the group consisting Streptomyces amphibiosporus and Streptomyces platensis. Generally various genes and proteins for the biosynthesis of glutarimide-containing polyketides, including iso-migrastatin or lactimidomycin gene cluster are described in U.S. Patent Publication US 2003/0171562, which is incorporated herein by reference for all purposes.

The invention further teaches methods for chemically modifying the glutarimide-containing polyketide analogs thus creating a library of glutarimide-containing polyketides analogs. In a preferred embodiment, the present invention provides a method of chemically modifying a glutarimide-containing polyketide analog said method comprising: (I) obtaining a glutarimide-containing polyketide analog, as described above; (II) conducting at least one of the modification to said glutarimide-containing polyketide analog to result in a chemically modified glutarimide-containing polyketide analog, wherein the modification is selected from the group consisting of: (a) a water mediated ring opening of the glutarimide-containing polyketide analog; (b) 1,4-addition of the glutarimide-containing polyketide analog by a sulfur-containing nucleophile, such as cysteine or β-mecaptoethanol, such as by Michael's additions or other methods known to one of ordinary skill in the art; (c) regioselective 1,4-reduction of the glutarimide-containing polyketide analog with agents such as Stryker's reagent, or other reducing reagents known to one of ordinary skill in the art; and (d) N-acylation of the glutarimide moiety of the glutarimide-containing polyketide analog; and (III) isolating and purifying at least one chemically modified glutarimide-containing polyketide analog.

Yet another embodiment of the invention provides a glutarimide-containing polyketide analog produced by the methods of described above and below in the Examples that follow.

Another embodiment of the present invention provides a pharmaceutical composition comprising a glutarimide-containing polyketide analog as described above; or its pharmaceutically-acceptable salt; and a pharmaceutically-acceptable carrier.

The present invention also provides an isolated, purified or enriched nucleic acid comprising a sequence selected from the group consisting of SEQ ID. Nos. 1, 2, 3 and 4; the sequences complimentary to SEQ ID. Nos. 1, 2, 3 and 4 and fragments comprising at least 10 consecutive nucleotides of the sequences complementary to SEQ ID. Nos. 1, 2, 3 and 4. In another embodiment, the present invention provides an isolated, purified or enriched nucleic acid capable of hybridizing to the nucleic acid comprising a sequence selected from the group consisting of SEQ ID. Nos. 1, 2, 3 and 4 under conditions of high stringency.

Certain other embodiments are described in the following examples. These examples are for illustrative purposes only and should not be deemed to limit the scope of the invention.

### EXAMPLE I

Migrastation (1) has emerged as a novel natural product lead for anticancer drug discovery because of its potent inhibitory effect on human tumor cell migration. (a) Nakae, K.; Yoshimoto, Y.; Sawa. T.; Homma, Y.; Hamada, M.; Takeuchi, T.; Imoto, M. J. Antibiot. 2000, 53, 1130-1136. (b) Takemoto, Y.; Nakae, K.; Kawatani, M.; Takahashi, Y.; Naganawa, H.; Imoto, M. J. Antibiot. 2001, 54, 1104-1107; Gaul, C., Njardarson, J. T., Shan, D.; Dorn, D. C.; Wu, K. D.; Tong, W. P.; Huang, X. Y.; Moore, M. A. S.; Danishefsky, S. J. J. Am. Chem. Soc. 2004, 26, 11326-11337 and references cited thereafter. Dorrigocin A (2) and B (3) were described as the first natural product inhibitors of the carboxyl methyltransferase involved in the processing of Ras-related proteins, serving as a valuable tool to study cellular signal transduction. Kadam, S.; McAlpine, J. B. J. Antibiot. 1994, 47, 875-880. Here we report that neither 1 nor 2 and 3 are nascent natural products but shunt metabolites of iso-migrastatin (4). These findings shed new light into the stability, stereochemistry, and biosynthetic relationship of this family of metabolites and should now be taken into consideration in evaluating their biological activities.

Imoto and co-workers first isolated 1 from Streptomyces sp. MK929-43F1 and revealed its structure as a 14-membered macrolide with a glutarimide side chain. See also Nakae, K.; Yoshimoto, Y.; Ueda, M.; Sawa, T.; Takahashi, Y.; Takeuchi, T.; Imoto, M. J. Antibiot. 2000, 53, 1228-1230. The relative and absolute stereochemistry of 1 was subsequently determined by X-ray crystallographic analysis. Nakamura, H.; Takahashi, Y.; Naganawa, H.; Nakae, K.; Imoto, M.; Shiro, M.; Matsumura, K; Watanabe, H.; Kitahara, T. J. Antibiot. 2002, 55, 442-444. Karwowski and co-workers first isolated 2 and 3 from S. platensis NRRL18993 and established their structures as glutarimide-containing linear polyketides. (a) Karwowski, J. P.; Jackson, M.; Sunga, G.; Sheldon, P.; Poddig, J. B.; Kohl, W. L.; Kadan, S. J. Antibiot. 1994, 47, 862-869. (b) Hochlowski, J. E.; Whittern, D. N.; Hill, P.; Mcalpine, J. B. J. Antibiot. 1994, 47, 870-874. The stereochemistry of 2 and 3 was not determined. Viewing 2 as an acyclic geometric isomer (at the C-11/C-12 double bond) of 1, Licari and co-workers re-investigated the fermentation of S. platensis and confirmed that this strain also produced 1, in additional to 2 and 3, as well as a new analog 4, which could be viewed as the cyclic form of 3. Woo, E. J.; Starks, C. M.; Carney, J. R.; Arslanin, R.; Cadapan, L.; Zavala, S.; Licari, P. J. Antibiot. 2002, 55, 141-146.The stereochemistry of 4 was not reported.

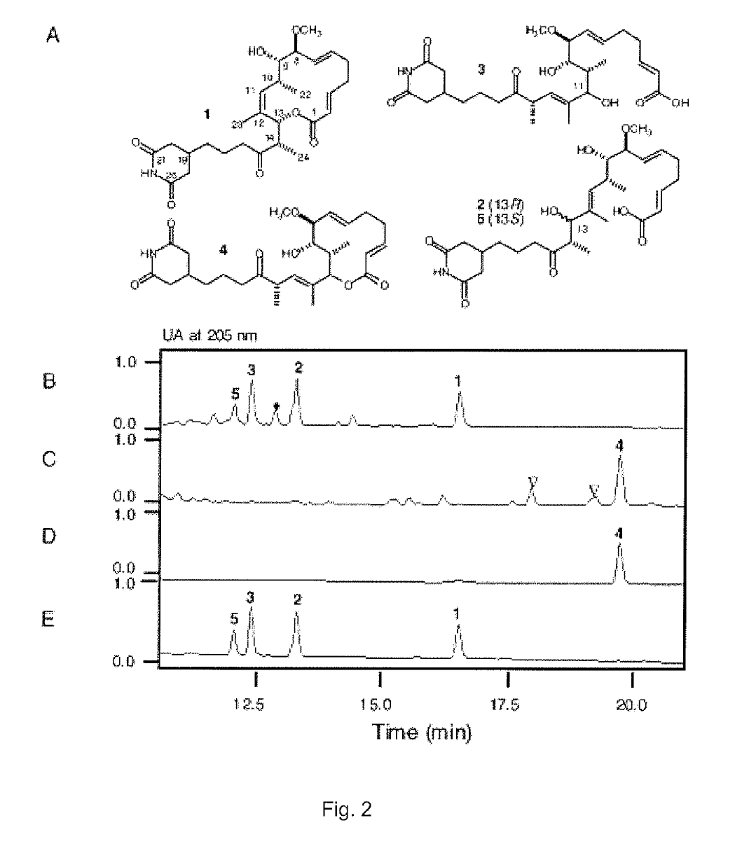

In our effort to clone and characterize secondary metabolite biosynthetic pathways in microorganisms, we identified multiple glutarimide-containing polyketide biosynthetic gene clusters in S. platensis. Du, L.; Sanchez, C.; Shen, B. Met. Engineer. 2001, 3, 78-95; Shen, B. Curr. Opinion Chem. Biol. 2003, 7, 285-295; Shen, B.; Liu, W.; Nonaka, K. Curr. Med. Chem. 2003, 10, 2317-2325. Surprisingly, inactivating one of the pathways abolished the production of all five metabolites (1 to 5), suggesting that these metabolites share the same biosynthetic machinery. This is unprecedented to polyketide biosynthesis, prompting us to further examine the fermentation behavior of this strain. We now present evidence supporting that 4 is the only nascent natural product biosynthesized by S. platensis and 1, 2, and 3 as well as a new member of this family, 13-epi-dorrigocin A (5) are shunt metabolites of 4 (FIG. 2A).

Fermentation was initially carried out as reported in the absence of resin. Nakae, K.; Yoshimoto, Y.; Sawa. T.; Homma, Y.; Hamada, M.; Takeuchi, T.; Imoto, M. J. Antibiot. 2000, 53, 1130-1136; Takemoto, Y.; Nakae, K.; Kawatani, M.; Takahashi, Y.; Naganawa, H.; Imoto, M. J. Antibiot. 2001, 54, 1104-1107; Nakae, K.; Yoshimoto, Y.; Ueda, M.; Sawa, T.; Takahashi, Y.; Takeuchi, T.; Imoto, M. J. Antibiot. 2000, 53, 1228-1230. HPLC and HPLC-MS analyses of the EtOAc extract of the fermentation broth indicated the presence of minimally five migrastatin and dorrigocin analogs (FIG. 2A), four of which were isolated for structural elucidation (˜60 mg/L). Extensive spectroscopic analyses confirmed that three of the four compounds are 1, 2 and 3, and compound 5 is a new analog that has a molecular formula of C₂₇H₄₁NO₈, identical to 2 and 3, upon HR-MALDI-MS analysis. A series of 2D NMR experiments (¹H—¹H COSY, TOCSY, HMQC, HMQC-TOCSY and gHMBC) for 5 and 2 allowed the full assignment of all ¹H and ¹³C NMR signals, establishing 5 as the C-13 epimer of 2.

Fermentation was then performed in the presence of resin (XAD-16) for its ability to sequester hydrophobic metabolites thereby increasing metabolite stability and production titer. Woo, E. J.; Starks, C. M.; Carney, J. R.; Arslanin, R.; Cadapan, L.; Zavala, S.; Licari, P. J. Antibiot. 2002, 55, 141-146. Strikingly, HPLC and HPLC-MS analyses of the MeOH eluent of XAD-16 resin harvested from the fermentation broth revealed a new set of metabolites (at least three) whose identities as migrastatin and dorrigocin analogs are apparent upon HR-MALDI-MS and NMR analyses, but the metabolites identified from fermentation in the absence of XAD-16 were surprisingly not detected at a significant level (FIG. 2B). We isolated the major metabolite (˜80 mg/L) and confirmed it to be 4 on the basis of extensive spectroscopic analysis. The sharp difference in metabolite profiles under the two fermentation conditions inspired us to further investigate the biosynthetic relationship among these metabolites.

We first verified that 1, 2, 3, and 5 are Table under the fermentation condition, and solutions of these compounds in CHCl₃, DMSO, MeOH, and MeOH—H₂0 kept at 25° C. for 90 days showed no detectable change upon HPLC and ¹H NMR analyses. In contrast, while relatively Table in anhydrous solvents such as DMF or DMSO, 4 undergoes rapid conversion to 1, 2, 3, and 5 in aqueous solution as exemplified in FIG. 2E. To verify the identity of the shunt metabolites, purified 4 (30 mg) was incubated in H₂O-DMSO (9:1), and the resultant products were isolated and subjected to spectroscopic analysis. The MS and ¹H NMR data as well as optical rotation data were identical to those of 1, 2, 3, and 5 isolated from fermentation.⁹ Taken together, these findings unambiguously demonstrate that 4 is the only nascent natural product produced by S. platensis and 1, 2, 3, and 5 are shunt metabolites of 4.

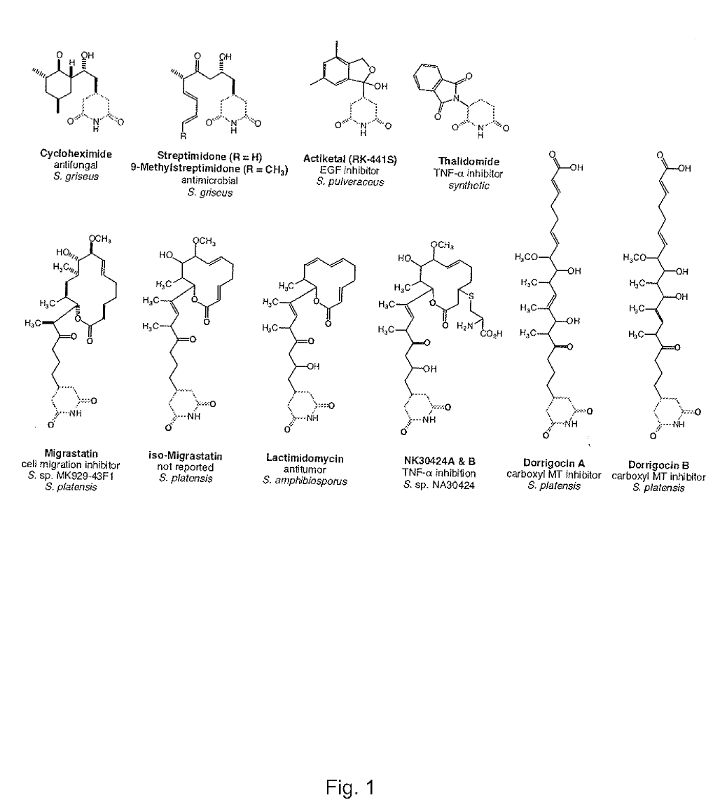

Since the absolute stereochemistry of 1 is known, conversion of 4 into 1, 2, 3, and 5 enabled us to conclude that 2, 3, 4, and 5 have the same configuration as 1 at C-8, -9, -10, and -14 (i.e., 8S, 9S, 10R, 14S for 2, 3, and 5 and 8S, 9S, 10S, 14S for 4) (FIG. 1A). This leaves the stereogenic centers at C-13 for 5 and 2 and at C-11 for 3 and 4 unassigned. While the latter are yet to be established, the configuration at C-13 for 5 and 2 can be deduced upon close inspection of their ¹H NMR data. Thus, conformation analysis by MM2 energy minimization revealed that the dihedral angle between H14-C14-C13-H13 is −48° and the —CH₃ group at C-24 and the —OH group at C-13 are in approximate syn-parallel disposition (80.8°) if C-13 assumes S configuration. In contrast, the dihedral angle between H14-C14-C13-H13 is −168.3°, and the —CH₃ group at C-24 and the —OH group at C-13 are in anti-parallel disposition (−170.9°) should C-13 assume R configuration. The former scenario would be consistent with the observed downfield shift of the —CH₃ group at C-24 (δ1.09) of 5 by 0.26 ppm compared with the analogous signal (δ0.83) of 2, as well as the splitting patterns and coupling constants of H13 (d, J=7.0 Hz in 5 and J=10.0 Hz in 2) and H14 (quintet, J=7.0 Hz in 5 and dq, J=10.0, 7.0 Hz in 2). On the basis of these analyses, the absolute stereochemistry of 2 (8S, 9S, 10R, 13R, 14S) and 5 (8S, 9S, 10R, 13S, 14S) were assigned, respectively (FIG. 2A).

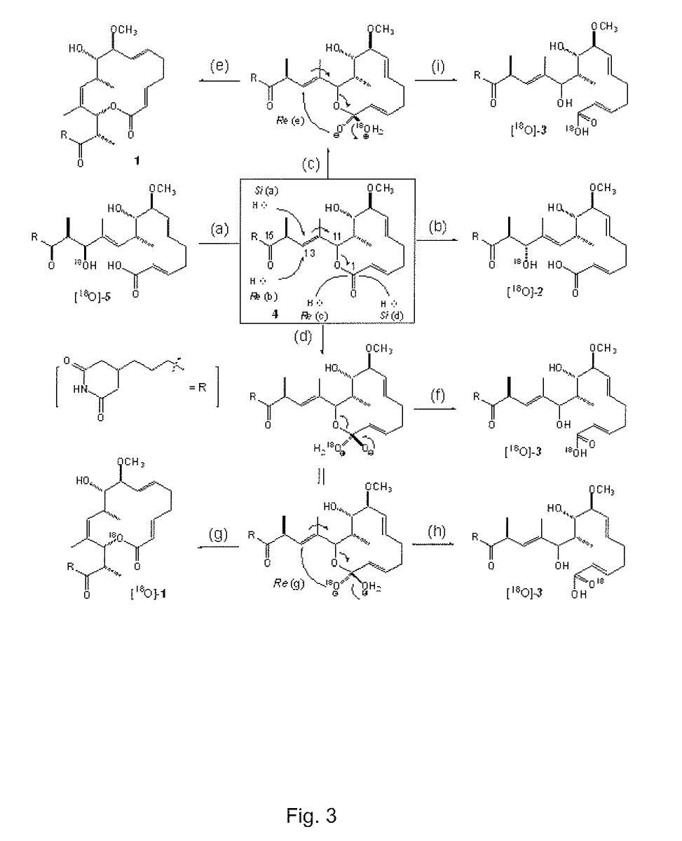

Isolation of 3 as single diastereomer and 2 and 5 as a pair of diastereomers suggests that the C—O bond at C-11 is intact during the hydrolysis of 4 to 3 (paths f, h, or i, FIG. 3) while 2 and 5 most likely result from H₂O attack at C-13 of 4 from either the Re or Si faces in an SN2′ mechanism (paths a and b, FIG. 3). Similarly, detection of 1 as the only diastereomer is indicative that the rearrangement of 4 to 1 must proceed in a Re face-specific, concerted mechanism (paths e or g, FIG. 3). To provide evidence for this mechanism, we carried out the reaction of 4 in H₂¹⁸O-DMSO (9:1), and the resultant products of 1, 2, 3, and 5 were subjected to HPLC-ESI-MS analysis to determine ¹⁸O incorporation. Two [M−H]⁻ ions at m/z of 488.2 and 490.3 (with a ratio of approximately 2:1 ) were observed for 1, corresponding to the incorporation of no and one ¹⁸O atom. This pattern agrees with the proposed mechanism, in which H₂¹⁸O attacks C-1 from the Re face (path c) and the resultant tetrahedral intermediate then undergoes Re face-specific rearrangement with the concomitant elimination of H₂¹⁸O (path e) to yield 1 with no net incorporation of ¹⁸O atom (FIG. 3). Incorporation of one ¹⁸O into 1 can be interpreted by either non-specific exchange of 1 (at C-15) in H₂¹⁸0 or H₂¹⁸O attacking C-1 from the Si face (path d) followed by the same Re face-specific rearrangement but with the concomitant elimination of H₂O (path g) to afford [¹⁸O]-1 (FIG. 3). In contrast, 3 yielded predominantly a [M−H]⁻ ion at m/z of 508.3 with an additional [M−H]⁻ ion at m/z of 510.2 with less than 10% of the intensity, representing the incorporation of one and two ¹⁸O atoms, respectively. The former agrees with paths f, h, or i (FIG. 3), while the latter can result from similar additional non-specific exchange in H₂¹⁸O. Finally, both 2 and 5 showed two [M−H]⁻ ions at m/z of 508.3 and 510.2 with a ratio of approximately 2:3, indicative of the incorporation of one and two ¹⁸O atoms, respectively. Again, the incorporation of one ¹⁸O atom into 5 and 2 agrees with paths a and b, respectively (FIG. 3), while additional non-specific exchange in H₂¹⁸O can account for the incorporation of two ¹⁸O atoms.

General Experimental Procedures.

Optical rotations were determined on a Perkin-Elmer 241 instrument at the sodium D line (589 nm). Electrospray ionization mass spectra (ESI−MS) and LC-MS data were obtained on an Agilent 1100 HPLC-MSD SL quadrupole mass spectrometer equipped with both orthogonal pneumatically assisted electrospray and atmospheric pressure chemical ionization sources. High-resolution (HR) MS analyses were acquired on an IonSpec HiResMALDI FT-Mass spectrometer. ¹H and ¹³C NMR spectra were recorded on Varian Unity Inova 500 MHz instruments operating at 500 MHz for ¹H and 125 MHz for ¹³C nuclei. ¹H and ¹³C NMR chemical shifts were referenced to residual solvent signals: δ_{H} 7.27 and δ_{C} 77.23 for CDCl₃ and δ_{H} 3.31 and δ_{C} 49.15 for MeOH-d₄. ¹H—¹H COSY, TOCSY (mixing time for 80 ms), HMQC, HMQC-TOCSY, and gHMBC (optimized ⁿJ_{XH} for 8.0 Hz) were performed using standard VARIAN pulse sequences. Silica gel 60A (Fisher Chemical 200-425 mesh) was used for flash column chromatography. Amberlite XAD-16 resin was purchased from Sigma. ¹⁸O-water (¹⁸O, >95%) was purchased from Cambridge Isotope Laboratories, Inc.

HPLC was carried out a Varian HPLC system equipped with ProStar 210 pumps and a photodiode detector. Mobile phases used were buffer A (15% CH₃CN in H₂O containing 0.1% HOAc) and buffer B (80% CH₃CN in H₂O containing 0.1% HOAc). Separation was achieved on a semi-preparative C18 column (Microsob C18, 250×10 mm, 5 pm), developed with a linear gradient from buffer A/buffer B (88:15) to buffer A/buffer B (10:90) in 20 min and continued at buffer A/buffer B (10:90) for an additional 5 min, at a flow rate of 2.5 ml/min and monitored by UV detection at 205 nm. Crude fermentation extracts, fractions obtained during isolation, reaction mixture, and the final purified compounds were all analyzed by HPLC (and LC-MS if necessary) on an analytical Cl 8 column (Prodigy ODS-2, 150×4.6 mm, 5 μm), developed with a linear gradient from buffer A/buffer B (100:0) to buffer A/buffer B (20:80) in 20 min and continued at buffer A/buffer B (20:80) for an additional 5 min, at flow rate of 1 ml/min and monitored by UV detection at 205 nm.

Bacterial Strain and Growth Condition. Streptomyces platensis NRRL18993 strain was grown on oatmeal agar plate (prepared by dissolving 3 g of oatmeal and 2 g of agar in 100 ml H₂O, boiling the resultant solution for 30 min, and autoclaving for 20 min) at 28° C. until it well sporulated (˜15 days). Spores were then harvested and stored in 20% glycerol at −80° C.

Fermentation, Extraction, and Isolation of Compounds 1, 2, 3, and 5. Considerable effort has been devoted to optimize the fermentation conditions of S. platensis NRRL18993 for metabolite production. The condition that produced the most amounts of metabolites was found to be the same as that reported for migrastatin (1) from the Streptomyces sp. MK9929-43F1 strain rather than the one used for 1 production from S. platensis. Nakae, K.; Yoshimoto, Y.; Sawa. T.; Homma, Y.; Hamada, M.; Takeuchi, T.; Imoto, M. J. Antibiot. 2000, 53, 1130-1136; Karwowski, J. P.; Jackson, M.; Sunga, G.; Sheldon, P.; Poddig, J. B.; Kohl, W. L.; Kadan, S. J. Antibiot. 1994, 47, 862-869; Hochlowski, J. E.; Whittern, D. N.; Hill, P.; Mcalpine, J. B. J. Antibiot. 1994, 47, 870-874; Woo, E. J.; Starks, C. M.; Carney, J. R.; Arslanin, R.; Cadapan, L.; Zavala, S.; Licari, P. J. Antibiot. 2002, 55, 141-146. A two-stage fermentation procedure was utilized. Seed medium (50 ml in 250-mL baffled flask) was inoculated with spores, and the flasks were incubated on a rotary shaker at 250 rpm and 28° C. for 2 days. This seed culture (50 ml) was then transferred into the fermentation medium (500 ml in 2-L baffled flask), and the flasks were incubated on a rotary shaker at 250 rpm and 28° C. for 5 days. Both seed and production media consisted of 2% glycerol, 2% dextrin, 1% soytone peptone, 0.3% yeast extract, 0.2% (NH₄)₂SO₄, 0.2% CaCO₃, pH 7.0, and were sterilized by autoclaving at 121° C. for 30 min.

The production fermentation culture (13 liters) was centrifuged (3,000 rpm at 4° C. for 16 min) to pellet the mycelia, and the broth was collected and filtrated to afford a clear supernatant. The latter was extracted twice with equal volumes of EtOAc. The combined EtOAc extracts were concentrated under reduced pressure to dryness to yield an oily residue (3.5 g). HPLC and LC-ESI-MS analysis of this crude mixture showed minimally three compounds with a [M−H]⁻ ion at m/z 506, corresponding to dorrigocin A (2), B (3) and 13-epi-dorrigocin A (5) and one compound with a [M−H]⁻ ion at m/z 488, corresponding to 1. Compounds 1, 2, 3, and 5 all showed end UV absorption.

This crude mixture was then subjected to silica gel flash column chromatography (3.5×40 cm), and the column was developed by stepwise elution with EtOAc-hexane containing 0.1% HOAc (from 3:7 to 10:0) to EtOAc-MeOH containing 0.1% HOAc (from 10:0 to 8:2), yielding 25 fractions each of which was analyzed by HPLC. Fractions 9-11 were combined and further purified by silica gel flash column chromatography (1.5×30 cm) eluted with CHCl₃-MeOH (from 10:0 to 9.5:0.5) followed by EtOAC-Hexane (from 4:6 to 10:0) to yield 1 (250 mg or 20 mg/L). Fraction 16 was subjected to silica gel flash column chromatography (1.5×30 cm) eluted with CHCl₃-MeOH (from 10:0 to 9.5:0.5) to give 3 (180 mg or 14 mg/L). Fractions 17-19 were combined and further resolved by semi-preparative HPLC to afford 2 (120 mg or 10 mg/L) and 5 (63 mg or 5 mg/L).

Fermentation, Extraction and Isolation of 4. The fermentation procedure for 4 was the same as that for compounds 1, 2, 3, and 5 except that the production medium contained 70 g/l XAD-16 resin, and fermentation time was increased to 10 days. The XAD-16 resins from the production fermentation culture (4 liters) were collected by filtration through gauze sponges and air-dried. The latter was then eluted with anhydrous ethanol and concentrated and concentrated in vacuo. The resultant residue was subjected to silica gel flash column chromatography (3.5×30 cm) eluted with CHCl₃-MeOH (from 10:0 to 9.5:0.5). The fraction eluted at CHCl₃-MeOH (9.7:0.3) was further resolved by repeated silica gel flash column chromatography (1.5×30 cm) developed by EtOAC-Hexane (from 4:6 to 10:0) to yield 4 (320 mg or 80 mg/L).

Physicochemical Properties of Compounds 1-5.

Migrastatin (1): Colorless oil; [α]²⁵_{D}+12.5° (c 3.18, MeOH); ¹H NMR, see Table 1; ¹³C NMR, see Table 2; ESI−MS, [M−H]⁻ ion at m/z488.3; HR-MALDI-MS, [M+Na]⁺ ion at m/z 512.2633 (calcd for [C₂₇H₃₉NO₇Na]⁺, 512.2624).

Dorrigocin A (2): Colorless oil; [α]²⁵_{D}+88.5° (c 0.15, MeOH); ¹H NMR See Table 1; ¹³C NMR, see Table 2; ESI−MS, [M−H]⁻ ion at m/z 506.3; HR-MALDI-MS, [M+Na]⁺ ion at m/z 530.2736 (calcd for [C₂₇H₄₁NO₈Na]⁺, 530.2730.

Dorrigocin B (3). Colorless oil; [α]²⁵_{D}+18.4° (c 0.14, MeOH); ¹H NMR See Table 1; ¹³C NMR, see Table 2; ESI−MS, [M−H]⁻ ion at m/z 506.3; HR-MALDI-MS, [M+Na]⁺ ion at m/z 530.2733 (calcd for [C₂₇H₄₁NO₈Na]⁺, 530.2730).

iso-Migrastatin (4). Colorless oil; [α]²⁵_{D} +123.9° (c 0.18, MeOH); ¹H NMR see Table 1; ¹³C NMR, see Table 2; ESI−MS, [M−H]⁻ ion at m/z488.3; HR-MALDI-MS, [M+Na]⁺ ion at m/z 512.2633 (calcd for [C₂₇H₃₉NO₇Na]⁺, 512.2624).

13-epi-Dorrigocin A (5). Colorless oil; [α]²⁵_{D}+14.6° (c 0.13, MeOH); ¹H NMR, see Table 1; ¹³C NMR, see Table 2; ESI−MS, [M−H]⁻ ion at m/z 506.3; HR-MALDI-MS, [M+Na]⁺ ion at m/z 530.2736 (calcd for [C₂₇H₄₁NO₈Na]⁺, 530.2730).

Large-scale Conversion of 4 to 1, 2, 3 and 5 in H₂O. The purity of compound 4 was first analyzed by HPLC showing a single peak (purity >98%) and then confirmed by ¹H NMR spectrum (in CDCl₃) showing no detectable contaminant. A solution of 4 (30 mg in 6 ml of DMSO) was then added to 55 ml Milli-Q H₂O, and the resultant mixture was incubated at 37° C. for 2 hours. The reaction mixture was evaporated under high vacuo. The residue was subjected to silica gel flash column chromatography eluted with CHCl₃-MeOH (from 10:0 to 9.4:0.6) to yield pure 1 (8 mg), 3 (9 mg), and a mixture of 2 and 5. The latter was further resolved by semi-preparative to afford pure 2 (6 mg) and 5 (4 mg). The ESI−MS, ¹H NMR and optical rotation data of compounds 1, 2, 3, and 5 were identical to those obtained from the corresponding compounds isolated from fermentation (the ¹³C NMR spectrum of 1 was also measured, which is identical to that of 1 obtained from fermentation).

Conversion of 4 to 1, 2, 3 and 5 in H₂¹⁸O. In a 1.5 ml microcentrifuge tube, 10 μl of 4 (10 mM in DMSO, purity >98%) and 90 pl of H₂¹⁸O were mixed and incubated at 37° C. for 2 hours. The resultant reaction mixture was lyophilized, and the residue was dissolved in 0.4 ml of MeOH for LC-MS analysis.

Table 1 depcits ¹H NMR (500 MHz) spectral data of migrastatin (1), dorrigocin A (2) and B (3), iso-migrastatin (4), and 13-epi-dorrigocin A (5)

TABLE 1

| Position | 1^{a,c} | 2^{b,d} | 3^{b,d} | 4^{a,c} | 5^{b,d} |
|---|---|---|---|---|---|
| 2 | 5.55(16.0, 1.5) | 5.83 d(15.5) | 5.85 d(15.5) | 5.69 d(16.0) | 5.86 d(15.0) |
| 3 | 6.47 ddd(16.0, | 6.96 dt(15.5, 7.0) | 6.96 dt(15.5, 6.5) | 6.65 ddd(16.0, 8.5, | 6.96 dt(15.0, |
| | 10.0, 3.5) | | | 7.5) | 6.5) |
| 4 | 2.20 m, 2.41 m | 2.36, 2H, m | 2.38, 2H, m | 2.15 m; 2.45 m | 2.37, 2H, m |
| 5 | 2.20 m, 2.41 m | 2.30, 2H, m | 2.32, 2H, m | 1.96 m, 2.60 m | 2.31, 2H, m |
| 6 | 5.50 ddd(15.5, | 5.73 dt(15.5, 6.5) | 5.79 dt(15.5, 6.5) | 5.60 ddd(15.5, | 5.76 dt(15.5, |
| | 10.0, 5.0) | | | 11.0, 4.5) | 6.5) |
| 7 | 5.22 dd(15.5, 5.0) | 5.51 brdd(16.0, | 5.25 brdd(15.5, 8.5) | 5.10(15.5, 3.5) | 5.50 dd(15.5, |
| | | 8.5) | | | 8.5) |
| 8 | 3.46 dd(8.5, 5.0) | 3.55 dd(8.5, 4.0) | 3.50 dd(8.5, 4.0) | 3.43 m | 3.46 dd(8.5, 4.0) |
| 9 | 3.02 dd(8.5, 1.5) | 3.20 dd(7.0, 4.0) | 3.36 dd(8.5, 2.0) | 3.75(9.5) | 3.13 dd(7.5, 4.0) |
| 10 | 2.91 m | 2.72 dq(10.0, 7.0) | 1.75 m | 1.85 m | 2.71 dq(10.0, |
| | | | | | 7.0) |
| 11 | 5.62 dd(10.5, 1.5) | 5.29 d(10.0) | 3.99 d(8.5) | 5.20(overlap) | 5.30 d(10.0) |
| 13 | 5.06 d(10.0) | 4.01 d(10.0) | 5.29 d(10.0) | 5.20(overlap) | 4.20 d(7.0) |
| 14 | 2.95 dd(10.0, 8.5) | 2.78 dq(10.0, 7.0) | 3.53 m | 3.43 m | 2.81 quintet(7.0) |
| 16 | 2.48, 2H, t(7.0) | 2.63, 2H, m | 2.51, 2H, m | 2.39m; 2.62 m | 2.58, 2H, m |
| 17 | 1.59, 2H, m | 1.62, 2H, m | 1.59, 2H, m | 1.59, 2H, m | 1.59, 2H, m |
| 18 | 1.32, 2H, m | 1.39, 2H, m | 1.37, 2H, m | 1.36, 2H, m | 1.37, 2H, m |
| 19 | 2.10 m | 2.14 m | 2.13 m | 2.10 m | 2.12 m |
| 20 | 2.20 m; 2.66 m | 2.31 m; 2.66 m | 2.32 m; 2.64 m | 2.25 m; 2.70 m | 2.31 m; 2.64 m |
| 22 | 0.94 d(7.0) | 0.97 d(6.5) | 0.93 d(7.0) | 0.87(7.5) | 0.98 d(6.5) |
| 23 | 1.83, 3H, d(1.5) | 1.64 d(1.0) | 1.67 d(1.0) | 1.90(1.5) | 1.64 d(1.0) |
| 24 | 1.10, 3H, d(8.5) | 0.83 d(7.0) | 1.13 d(7.0) | 1.14(7.0) | 1.09 d(7.0) |
| 25 | 2.20 m; 2.66 m | 2.31 m; 2.66 m | 2.32 m; 2.64 m | 2.25 m; 2.70 m | 2.31 m; 2.64 m |
| 8-OCH₃ | 3.28, 3H, s | 3.22 s | 3.26 s | 3.34 s | 3.20 s |

^{a}Signals were assigned with aid of ¹H—¹H COSY and HMQC experiments.
^{b}Signals were assigned with aid of ¹H—¹H COSY, TOCSY, HMQC, HMQC-TOCSY, and gHMBC experiments.
^{c}Sample measured in CDCl₃.
^{d}Sample measured in CD₃OD.

Table 2 depicts ¹³C NMR (125 MHz) spectral data of migraststin (1) dorrigocin A (2) and B (3), iso-migraststin (4), and 13-epi-dorrigocin A (5)

TABLE 2

| Position | 1^{a,c} | 2^{b,d} | 3^{b,d} | 4^{a,c} | 5^{b,d} |
|---|---|---|---|---|---|
| 1 | 164.0 | 170.1 | 170.0 | 167.8 | 170.0^{e} |
| 2 | 122.3 | 123.4 | 123.3 | 125.2 | 123.8^{e} |
| 3 | 150.1 | 150.2 | 150.2 | 150.8 | 150.3 |
| 4 | 30.5 | 32.8 | 32.8 | 30.3* | 32.9 |
| 5 | 31.2 | 32.0 | 32.0 | 32.9 | 32.0 |
| 6 | 130.6 | 135.3 | 136.6 | 129.3 | 135.3 |
| 7 | 128.1 | 130.4 | 129.1 | 130.5 | 130.5 |
| 8 | 82.7 | 84.5 | 86.3 | 81.8 | 84.4 |
| 9 | 78.0 | 79.1 | 75.8 | 73.4 | 79.2 |
| 10 | 32.1 | 36.0 | 38.3 | 38.3 | 35.9 |
| 11 | 133.2 | 133.7 | 80.7 | 82.4 | 131.6 |
| 12 | 131.3 | 136.0 | 140.3 | 134.3 | 136.3 |
| 13 | 77.1 | 82.0 | 128.0 | 128.2 | 78.6 |
| 14 | 51.2 | 50.4 | 47.3 | 46.2 | 51.5 |
| 15 | 211.0 | 216.7 | 213.9 | 211.0 | 215.0 |
| 16 | 40.3 | 43.8 | 41.5 | 40.1 | 42.1 |
| 17 | 20.3 | 21.2 | 21.7 | 20.5 | 21.6 |
| 18 | 34.3 | 35.3 | 35.4 | 34.4 | 35.4 |
| 19 | 30.2 | 31.6 | 31.6 | 30.3* | 31.8 |
| 20, 25 | 37.8* | 38.7 | 38.6 | 37.9 | 38.7 |
| 21, 26 | 172.5 | 175.7 | 175.7 | 172.3 | 175.6 |
| 22 | 13.5 | 16.5 | 8.6 | 10.7 | 16.4 |
| 23 | 26.1 | 10.9 | 12.4 | 13.5 | 13.0 |
| 24 | 13.5 | 14.0 | 16.6 | 15.9 | 12.4 |
| 8-OCH₃ | 57.0 | 56.5 | 56.7 | 57.3 | 56.5 |

^{a}Signals were assigned with aid of ¹H—¹H COSY and HMQC experiments.
^{b}Signals were assigned with aid of ¹H—¹H COSY, TOCSY, HMQC, HMQC-TOCSY, and gHMBC experiments.
^{c}Samples measured in CDCl₃.
^{d}Samples measured in CD₃OD.
^{e}Not observed in ¹³C NMR spectrum but observed as a cross-peak in HMQC or gHMBC spectra.
*Overlapped signals.

### EXAMPLE II

Iso-Migrastatin Congeners from Streptomyces platensis and Generation of a Glutarimide Polyketide Library Featuring the Dorrigocin, Lactimidomycin, Migrastatin, and NK30424 Scaffolds

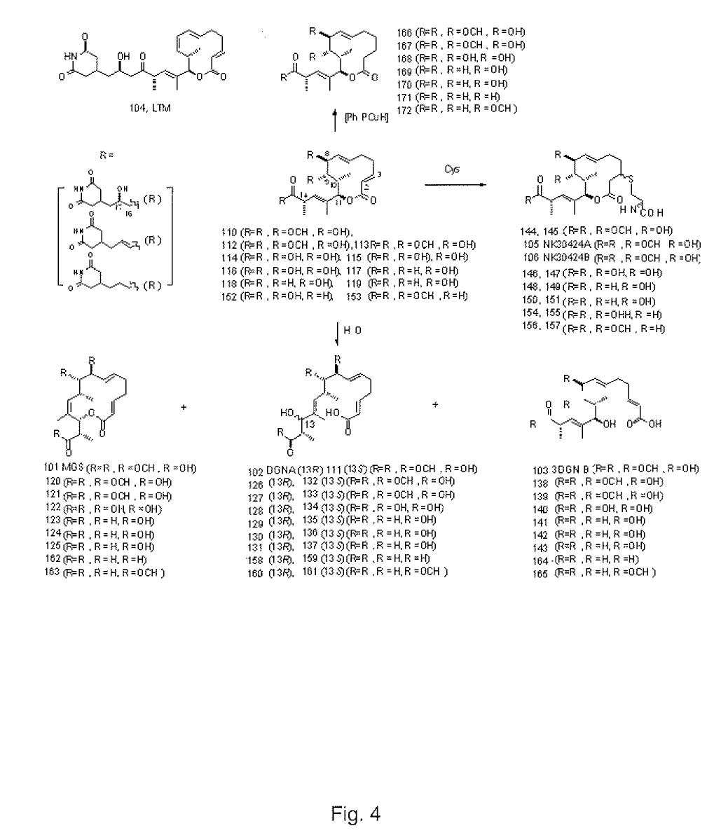

As discussed above, and further as shown in FIG. 4, Migrastatin (101) is a tumor cell migration specific inhibitor and synthetic analogs of 101 have been prepared as therapeutic candidates for treating tumor metastasis. Gaul, C.; Njardarson, J. T.; Shan, D.; Dorn, D. C.; Wu, K. D.; Tong, W. P.; Huang, X. Y.; Moore, M. A.; Danishefsky, S. J. J. Am. Chem. Soc. 2004, 126, 11326-11337; Shan, D.; Chen, L.; Njardarson, J. T.; Gaul, C.; Ma, X.; Danishefsky, S. J.; Huang, X.-Y. Proc. Natl. Acad. Sci. USA. 2005, 102, 3772-3776 and references cited therein. Dorrigocin A (102) and B (103) are novel natural product inhibitors of carboxyl methyltransferase involved in the processing of Ras-related proteins, serving as a valuable tool to study cellular signal transduction. Structurally related to 101, 102, and 103 are lactimidomycin (104) and NK30424 A (105) and B (106). Sugawara, K.; Nishiyama, Y.; Toda, S.; Komiyama, N.; Hatori, M.; Moriyama, T.; Sawada, Y.; Kamei, H.; Konishi, M.; Oki, T. J. Antibiot. 1992, 45, 1433-1441; Takayasu, Y.; Tsuchiya, K.; Aoyama, T.; Sukenaga, Y. J. Antibiot. 2001, 54, 1111-1115; Takayasu, Y.; Tsuchiya, K.; Sukenaga, Y. J. Antibiot. 2002, 55, 337-340. While 104 exhibits potent antitumor activity, 105 and 106 inhibit LPS-induced TNF-α production by suppressing the NF-κB signaling pathway, a property that could be exploited for the treatment of cancer and inflammation. These natural products belong to the glutarimide polyketide family, which also include antifungal antibiotic cycloheximide (107), streptimidone (108), and 9-methylstreptimidone (109). Allen, M. S.; Becker, A. M.; Rickards, R. W. Aust. J. Chem. 1976, 29, 673-679; Jeffs, P. W.; McWilliams, D. J. Am. Chem. Soc. 1981, 103, 6185-6192. (c) Kim, B. S.; Moon, S. S.; Hwang, B. K. J. Agric. Food Chem. 1999, 47, 3372-3380 and references cited therein.

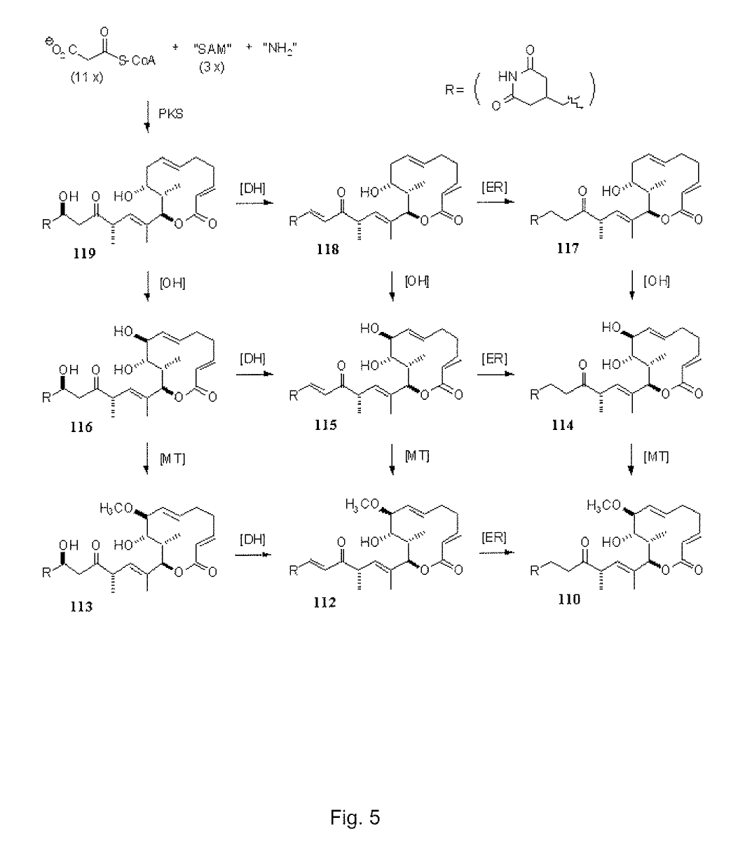

As discussed above, iso-migrastatin (110) is the main natural product of Streptomyces platensis NRRL18993 and 101, 102, and 103 as well as 13-epi-dorrigocin A (111) can be readily derived from 110 via a facile, H₂O-mediated rearrangement. Ju, J.; Lim, S.-K.; Jiang, H.; Shen, B. J. Am. Chem. Soc. 2005, 127, 1622-1623. Here we report the isolation and structural elucidation of eight new congeners (112-119) of 110 and generation of a forty seven-member library of glutarimide polyketides featuring the molecular scaffolds of 101-106, 110, and 111 (FIG. 4), setting the stage to investigate the structure and activity relationship for this family of natural products. These results also established the absolute stereochemistry of 105 and 106 and shed new light into the post-polyketide synthase (PKS) steps for the biosynthesis of 110 (FIG. 5).

In our early effort to characterize 110 from S. platensis fermentation, we noticed a plethora of minor metabolites whose identity as biosynthetic congeners of 110 is apparent upon HPLC-UV and LC-ESI-MS analyses. Ju, J.; Lim, S.-K.; Jiang, H.; Shen, B. J. Am. Chem. Soc. 2005, 127, 1622-1623. Since the relative abundance of these congeners appeared to be fermentation condition dependent, we optimized their production by altering the age of the seed culture, level of dissolving oxygen, time of production fermentation, and nature of hydrophobic resin (XAD-16 vs HP-20) supplemented, as discussed below. Under the optimized conditions, S. platensis was typically fermented in the presence of XAD-16 for its ability to sequester, hence stabilize the metabolites and increase their production titer. The resin was harvested by filtration and air-dried, and the metabolites were eluted with anhydrous ethanol and concentrated in vacuo to afford a crude mixture. Purification of these compounds proved to be challenging, since they undergo a rapid, H₂O-mediated rearrangement, precluding any aqueous solvent-based preparative HPLC method. They were eventually separated by repeated flash silica gel column chromatography, eluted alternatively with two different solvent systems (CHCl₃/MeOH and hexane/EtOAc), and water was avoided throughout the purification. These procedures resulted in the identification of eight new compounds (112-119), together with 110 as the major metabolite (FIG. 4). With the exception of 115 and 116, the isolated yields of the new compounds range from 5-35 mg/L.

The ¹H and ¹³C NMR spectra of the purified compounds were fully assigned on the basis of extensive 1D and 2D NMR (COSY, TOCSY, HMQC, and gHMBC) and ESI−MS and high-resolution MALDI-FT-MS analyses. These studies established the structures of 112-115 and 117-119 as shown in FIG. 4. Although we were not able to purify 116 due to its low abundance, its structure was supported by LC-ESI-MS analysis; its short HPLC retention time is also consistent with 116 being the most polar congener among the nine compounds identified.

The stereochemistry of these compounds was deduced from their ¹H and ¹³C NMR spectra data in comparison with those of 110. The C-16/C-17 trans double bond (for 112, 115 and 118) was based on its diagnostic coupling constant. The HO— group at C-17 (for 113 and 119) was assigned R configuration on the basis of its near identical splitting patterns and chemical shifts at C-15, C-16, C-17, C-18, and C-19 to those of 108 (all measured in CDCl₃), the absolute R configuration of which had been determined previously. Allen, M. S.; Becker, A. M.; Rickards, R. W. Aust. J. Chem. 1976, 29, 673-679; Jeffs, P. W.; McWilliams, D. J. Am. Chem. Soc. 1981, 103, 6185-6192; Kim, B. S.; Moon, S. S.; Hwang, B. K. J. Agric. Food Chem. 1999, 47, 3372-3380 and references cited therein. Other asymmetric carbon centers (8S, 9S, 10S, 11R, and 14S) were postulated to be identical to those of 110 by comparative analyses of their ¹H and ¹³C NMR splitting patterns and chemical shifts. Ju, J.; Lim, S.-K.; Jiang, H.; Shen, B. J. Am. Chem. Soc. 2005, 127, 1622-1623; Woo, E. J.; Starks, C. M.; Carney, J. R.; Arslanian, R.; Cadapan, L.; Zavala, S.; Licari, P. J. Antibiot. 2002, 55, 141-146. The isolation of these congeners not only further supports the early conclusion of 110 as the main metabolite of S. platensis but also provides new insight into the post-PKS steps for the biosynthesis of 110. Four minimal steps, hydroxylation at C-8, O-methylation at HO—C-8, dehydration at HO—C-17/C-16, and enoyl reduction at C-16/C-17, could be conceptually envisaged to convert the nascent polyketide intermediate 119 to 110 (FIG. 5). The fact that all possible intermediates have been identified strongly supports this proposal. The current study unveiled the substrate promiscuity of the enzymes for the post-PKS steps, a property that could be exploited to further expand the structural diversity for this family of natural products by processing additional analogs with modified polyketide scaffolds.

The availability of the new congeners inspired us to investigate if they can undergo the same H₂O-mediated rearrangement into the corresponding 101, 102, 103, and 111 scaffolds as 110. Incubation of 112, 113, 114, 117, 118, or 119 in H₂O indeed afforded, in quantitative yield, the corresponding congeners of 101 (120-125), 102 (126-131), 111 (132-137), and 103 (138-143), respectively, whose structures were confirmed upon extensive 1D and 2D NMR as well as MS analyses (FIG. 4).

Isolation of 113 also prompted us to examine the biosynthetic relationship between 101 and 105 and 106. Takayasu and co-workers first isolated 105 and 106 from Streptomyces sp. NA30424 and established their structures as two stereoisomers on the basis of NMR and MS data, with their stereochemistry undetermined. Takayasu, Y.; Tsuchiya, K.; Aoyama, T.; Sukenaga, Y. J. Antibiot. 2001, 54, 1111-1115; Takayasu, Y.; Tsuchiya, K.; Sukenaga, Y. J. Antibiot. 2002, 55, 337-340. Viewing 105 and 106 as a cysteine 1,4-adduct at C-2/C-3 of 113, we incubated 113 with cysteine. The reaction was completed in 2 minutes at room temperature, yielding quantitatively two diasteromers at C-3 in an approximately 2:3 ratio whose identity as 115 and 116 was confirmed by their identical ¹H and ¹³C NMR spectra to those reported previously. Since all chiral centers in 113 remain intact during the 1,4-Michael addition, this result also established the absolute stereochemistry of 105 and 106 (i.e., the same configuration of 8S, 9S, 10S, 11R, 14S, and 17R as in 113). The apparent intrinsic reactivity of the C-2/C-3 double bond as Michael acceptor further inspired us to test if 110 and its other congeners (114, 117, and 119) can undergo the same 1,4-addtion. Incubation of these compounds with cysteine indeed afforded the corresponding adducts in quantitative yields, the structures of which as 144-151 were established by MS and 1D and 2D NMR analyses (FIG. 4). Rapid addition was also observed between cysteine and 112, 115, or 118, but yielded a mixture of up to four adducts due to the presence of the second Michael acceptor at C-16/C-17. This facile reactivity also raises the question if 113 is the main metabolite of S. sp. NA30424 and 105 and 106 in fact are the result of adventitious addition between 113 and cysteine during fermentation or isolation.

Synthetic analogs of 101, 102, 103, 105, and 106 with improved biological activities have been prepared, validating the utility of this privileged molecular scaffold in lead optimization. Gaul, C.; Njardarson, J. T.; Shan, D.; Dorn, D. C.; Wu, K. D.; Tong, W. P.; Huang, X. Y.; Moore, M. A.; Danishefsky, S. J. J. Am. Chem. Soc. 2004, 126, 11326-11337; Shan, D.; Chen, L.; Njardarson, J. T.; Gaul, C.; Ma, X.; Danishefsky, S. J.; Huang, X.-Y. Proc. Natl. Acad. Sci. USA. 2005, 102, 3772-3776 and references cited therein; Takayasu, Y.; Tsuchiya, K.; Aoyama, T.; Sukenaga, Y. J. Antibiot. 2001, 54, 1111-1115; Takayasu, Y.; Tsuchiya, K.; Sukenaga, Y. J. Antibiot. 2002, 55, 337-340. None of the compounds produced in this study have been prepared previously, however, and nor can they be readily accessed via total synthesis. The current study therefore complements the synthetic effort and opens an alternative way to further diversify the glutarimide polyketide molecular scaffold. Combination of both biosynthetic and synthetic efforts holds the greatest promise to fully realize the potential of this family of natural products in drug discovery.

General Experimental Procedures.

¹H and ¹³C NMR spectra were recorded at 25° C. on Varian Unity Inova 500 instruments operating at 500 MHz for ¹H and 125 MHz for ¹³C nuclei. ¹H and ¹³C NMR chemical shifts were referenced to residual solvent signals: δ_{H} 7.27 and δ_{C} 77.23 for CDCl₃, and δ_{H} 3.31 and δ_{C} 49.15 for MeOH-d₄. ¹H—¹H COSY, ¹H—¹H TOCSY (mixing time=80 ms), HMQC (¹J_{CH}=140 Hz), and gHMBC (²⁻³J_{XH}=8.0 Hz) were performed using standard VARIAN pulse sequences. Electrospray ionization-mass spectrometry (ESI−MS) and LC-MS were carried out on an Agilent 1100 HPLC-MSD SL quadrupole mass spectrometer equipped with both orthogonal pneumatically assisted electrospray and atmospheric pressure chemical ionization sources. High-resolution MS analyses were acquired on an IonSpec HiResMALDI FT-Mass spectrometer with a 7 tesla superconducting magnet. A saturated solution of 2,5-dihydroxybenzoic acid in methanol was used for matrix preparation, and the spectra were peak-matched using m/z 273.03936 ([2M-2H₂O+H]⁺) as a reference peak. Optical rotations were measured in CHCl₃ on a Perkin-Elmer 241 instrument at the sodium D line (589 nm).

HPLC was carried out on a Varian system equipped with Prostar 210 pumps and a photodiode array (PDA) detector. Unless otherwise stated, the mobile phase used comprises of buffer A (15% CH₃CN in H₂O containing 0.1% HOAc) and buffer B (80% CH₃CN in H₂O containing 0.1% HOAc). Analytical HPLC and LC-MS was conducted using a Prodigy ODS-2 column (150×4.6 mm, 5 μm, Phenomenex, Torrance, Calif.) eluted with a linear gradient of 100% buffer A and 0% buffer B to 20% buffer A and 80% buffer B over 20 min, followed by 5 min at 20% buffer A and 80% buffer B at a flow rate of 1.0 mL/min with UV detection at 205 nm. Semi-preparative HPLC was conducted using a C18 column (250×10 mm, 5 μm, Microsob, Varian) eluted with a linear gradient of 80% buffer A and 20% buffer B to 10% buffer A and 90% buffer B over 20 min, followed by 10 min at 10% buffer A and 90% buffer B at a flow rate of 2.5 mL/min with UV detection at 205 nm.

Silica gel 60A (200-425 mesh, Fisher Chemical) was used for flash column chromatography. Amberlite XAD-4, XAD-16, and Diaion HP-20 resins were purchased from Sigma. Crude fermentation extracts, fractions obtained during isolation, compounds finally purified, and reaction mixtures were all monitored by either analytical HPLC or TLC on E. Merck silica gel-60F plates using CHCl₃/MeOH (9:1) or EtOAc/Hexane (7:3) as developing solvents and iodide as visualizing agent.

Examination of the metabolite profiles of Streptomyces platensis NRRL18993 under different fermentation conditions by HPLC and LC-MS analyses. It is well known that the culture conditions (e.g. medium composition, aeration, culture vessel, temperature, pH, etc.) can affect the secondary metabolite profiles of the microorganism. The relative abundance of each component in the mixture of metabolites obtained from S. platensis cultures was found to be variable, depending upon the culture conditions. Our work has been focusing on further optimizing the culture condition to improve the titers of the whole set of metabolites. The general fermentation procedure utilized was a two-stage process. Spore suspension (50 μL) of S. platensis was inoculated into 50 mL seed medium in a 250-mL flask. The seed culture was incubated on a rotary shaker at 250 rpm and 28° C. for 30-48 hr. The resultant seed culture (2.5 mL, i.e., 5% of the production medium) was inoculated into 50 mL production medium in a 250-mL flask, which was incubated on a rotary shaker at 250 rpm and 28° C. for 1.5-10 days. The seed medium consisted of 2% glycerol, 2% dextrin, 1% soytone peptone, 0.3% yeast extract, 0.2% (NH₄)₂SO₄, and 0.2% CaCO₃, pH 7.0. The production medium contained the same ingredients as those in seed medium but with additional 20-150 g/L resins. Both the seed and production media were sterilized for 30 min at 121° C. in an autoclave.

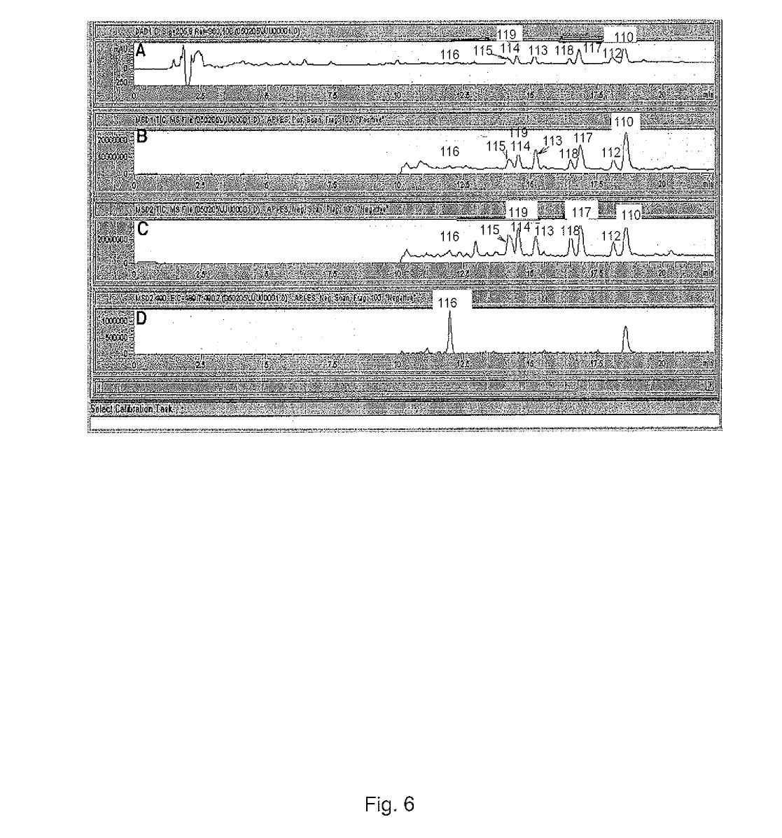

Metabolite titers were optimized by altering the seed culture, amount of adsorbent resins supplemented, duration of fermentation time, and level of oxygen supplied. Highest yields were obtained by the combination of (a) relatively young seed culture (30 hr), (b) production medium supplemented with high percentage of resins, (c) relatively short fermentation time, and (d) reduced oxygen supply. Thus, no adverse effect on S. platensis growth was observed upon supplementation to the production medium with 2%-15% (w/v) XAD-16 or 2%-7% (w/v) HP-20 resins. While 110 is the major metabolite during the course of 4 to 10 day fermentation, the relative abundance of the other congeners (112-119) to 110 increases dramatically when the fermentation is terminated in 1.5-2 days. Increasing the volume of the production medium in a fixed size flask, thereby reducing the relative level of dissolving oxygen often resulted in an increased amount of 112-119 relative to 110. Further restriction of dissolving oxygen by covering the flask with alumina foil afforded 117-119 as the predominant metabolites. In summary, the highest yields for 110 and 112-119 were obtained when 50 mL production medium in a 250-mL flask supplemented with 15% XAD-16 or 7% HP-20 resins was cultured for 42-48 hr (FIG. 6).

Large-scale fermentation, isolation, and structure elucidation of 110 and its new congeners 112-119.

Seed medium (50 mL) in a 250-mL flask was inoculated with spores of S. platensis and incubated on a rotary shaker at 250 rpm and 28° C. for 30 hr. The resultant seed culture (25 mL) was then inoculated into production medium (600 mL containing 70 g/L XAD-16 resins) in a 2-L flask, which was incubated on a rotary shaker at 250 rpm and 28° C. for 2 days. The combined production culture broth (7 L) was filtered through gauze sponges to capture the XAD-16 resins. The resins were washed with Milli-Q H₂O, dried at room temperature, and eluted with anhydrous ethanol. The ethanol extract was evaporated under reduced pressure to yield an oily residue. The residue was first analyzed by HPLC-UV (PDA) and LC-ESI-MS to confirm the presence of the various congeners of 110. Although they can survive during fermentation and be stabilized in H₂O upon absorption to resins, 110 and its congeners 112-119 undergo rapid rearrangement in aqueous solution. They are stable in most organic solvents such as CHCl₃, EtOAc, hexane, and absolute ethanol at room temperature. Consequently, isolation of these metabolites was performed mainly by repeated silica gel flash column chromatography using two different solvent systems (CHCl₃/MeOH and EtOAc/hexane).

The oily residue was initially applied on a silica gel flash column (3.8×45 cm), eluted with a CHCl₃/MeOH (10:0-9:1) gradient, roughly yielding six fractions (A-F), on the basis of analytical HPLC. Fraction A was subjected to silica gel flash column chromatography (3.8×45 cm) using a CHCl₃/MeOH (10:0-9.7:0.3) gradient elution to afford A1 (containing mainly 10) and A2 (containing 110 and 112). Fraction A1 was further purified by silica gel flash column chromatography (3.8×45 cm) using a EtOAc/hexane (4:6-8:2) gradient elution, giving pure compound 110 (550 mg). Fraction A2 was separated repeatedly by series of silica gel flash column chromatography (2.5×30 cm, 1.9×30 cm, 1.3×30 cm), eluted with a CHCl₃/MeOH (10:0-9.7:0.3) or a EtOAc/hexane (4:6-8:2) gradient to afford pure 112 (30 mg). Similarly, further separation of fraction B upon elution with a CHCl₃/MeOH (10:0-9.6:0.4) or a EtOAc/Hexane (4:6-9:1) gradient led to pure 117 (230 mg) and 118 (35 mg), of fraction C upon elution with a CHCl₃/MeOH (10:0-9.5:0.5) or a EtOAc/hexane (5:5-9:1 ) gradient essentially provided pure 113 (65 mg), and of fraction D upon elution with a CHCl₃/MeOH (10:0-9.4:0.6) or a EtOAc/hexane (5:5-10:0) gradient resulted in 114 (70 mg) and 119 (45 mg) in pure form and a fraction D3 (30 mg) that showed a single broad peak from analytical HPLC but contained both 115 and 119 as evidenced by ¹³C NMR spectral data. Attempts in obtaining high purity form of 115 were unsuccessful due to its relative low abundance and the very close polarity between 115 and 119. However, it was possible to prepare two batch of samples enriched in 115 and 119 at different ratio (approximate 1:3 and 3:1), which enabled us to solve the structure of 115 via analyses of 1D and 2D NMR and MS spectra. Due to its extremely low abundance, 116 was only detected by LC-ESI-MS but not purified (FIG. 6). It should be pointed out that the yields reported are isolated yields for 110 and its congeners. Given the difficulty in their purification, these yields most likely are underestimated, and their actual titers in S. platensis should be even higher.

Structure Elucidation of Compounds 112-119.

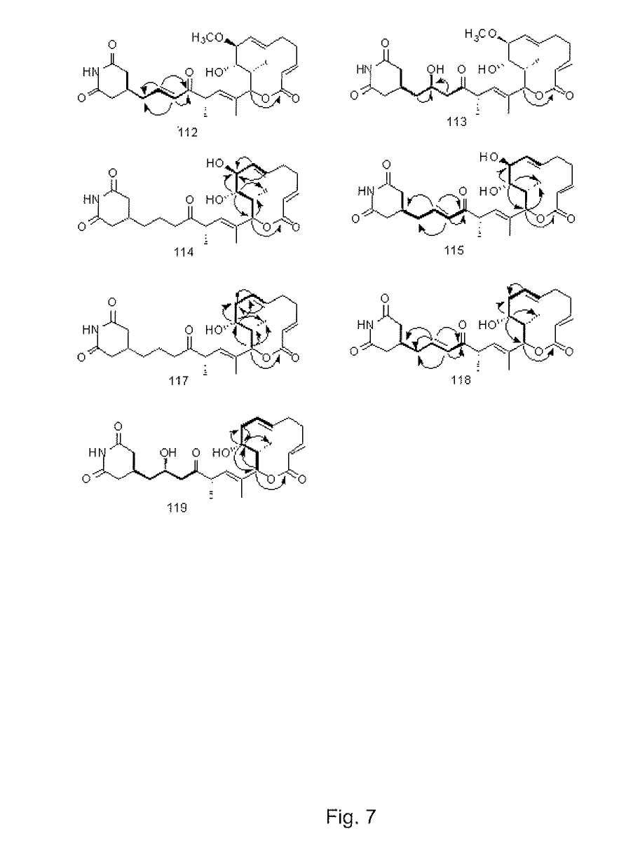

16,17-Didehydro-isomigrastatin (112): Colorless oil; [α]²⁵_{D}+123.9° (c0.18, CHCl₃); ¹H NMR, see Table 3; ¹³C NMR, see Table 5; selected COSY and gHMBC correlations, see FIG. 7; ESI−MS (−), m/z 486.1 ([M−H]⁺); ESI−MS (+), m/z 488.2 ([M+H]⁺), 510.1 ([M+Na]⁺); HR-MALDI-MS (+), m/z 510.2500 ([M+Na]⁺) (calcd 510.2468 for [C₂₇H₃₇O₇N Na]⁺).

17-Hydroxy-isomigrastatin (113): Colorless oil; [α]²⁵_{D}+140.0° (c0.10, CHCl₃); ¹H NMR, see Table 3; ¹³C NMR, see Table 5; selected COSY and gHMBC correlations, see FIG. 7; ESI−MS (−), m/z 504.2 ([M−H]⁺); ESI+MS (+), m/z 506.1 ([M+H]⁺), 528.0 ([M+Na]⁺); HR-MALDI-MS (+), m/z 528.2550 ([M+Na]⁺) (calcd 528.2573 for [C₂₇H₃₉O₈N Na]⁺).

8-Desmethyl-isomigrastatin (114): Colorless oil; [α]²⁵_{D}+83.0° (c0.10, CHCl₃); ¹H NMR, see Table 3; ¹³C NMR, see Table 5; selected COSY and gHMBC correlations, see FIG. 7; ESI−MS (−), m/z 474.2 ([M−H]⁺); ESI−MS (+), m/z 476.0 ([M+H]⁺), 498.0 ([M+Na]⁺); HR-MALDI-MS (+), m/z 498.2476 ([M+Na)⁺) (calcd 498.2440 for [C₂₆H₃₇O₆N Na]⁺).

16, 17-Didehydro-8-desmethyl-isomigrastatin (115): Colorless oil; 1H NMR, see Table 4; ¹³C NMR, see Table 5; selected COSY and gHMBC correlations, see FIG. 7; ESI−MS (−), m/z 472.2 ([M−H]⁺); ESI−MS (+), m/z 473.9 ([M+H]⁺), 496.0 ([M+Na])⁺.

17-Hydroxy-8-desmethyl-isomigrastatin (116): ESI−MS (−), m/z490.2 ([M−H]⁺); ESI−MS (+), m/z 492.1 ([M+H]⁺).

8-Desmethoxy-isomigrastatin (117): Colorless oil; [α]²⁵_{D}+77.0° (c0.10, CHCl₃); ¹H NMR, see Table 4; ¹³C NMR, see Table 5; selected COSY and gHMBC correlations, see FIG. 7; ESI−MS (−), m/z 458.3 [M−H]⁺; ESI−MS (+), m/z460.1 ([M+H]⁺), 482.0 ([M+Na]⁺); HR-MALDI-MS (+), m/z 482.2528 ([M+Na]⁺) (calcd 482.2519 for [C₂₆H₃₇O₆N Na]⁺).

16,17-Didehydro-8-desmethoxy-isomigrastatin (118): Colorless oil; [α]²⁵_{D}+7.3° (c0.05, CHCl₃); ¹H NMR, see Table 4; ¹³C NMR, see Table 5; selected COSY and gHMBC correlations, see FIG. 7; ESI−MS (−), m/z 456.2 ([M−H]⁺); ESI−MS (+), m/z 458.0 ([M+H]⁺), 480.0 ([M+Na]⁺).

17-Hydroxy-8-desmethoxy-isomigrastatin (119): Colorless oil; [α]²⁵_{D}+80.0° (c0.10, CHCl₃); ¹H NMR, see Table 4; ¹³C NMR, see Table 5; selected COSY and gHMBC correlations, see FIG. 7; ESI−MS (−), m/z 474.2 ([M−H]⁺); ESI−MS (+), m/z 476.0 ([M+H]⁺), 498.0 ([M+Na]⁺).

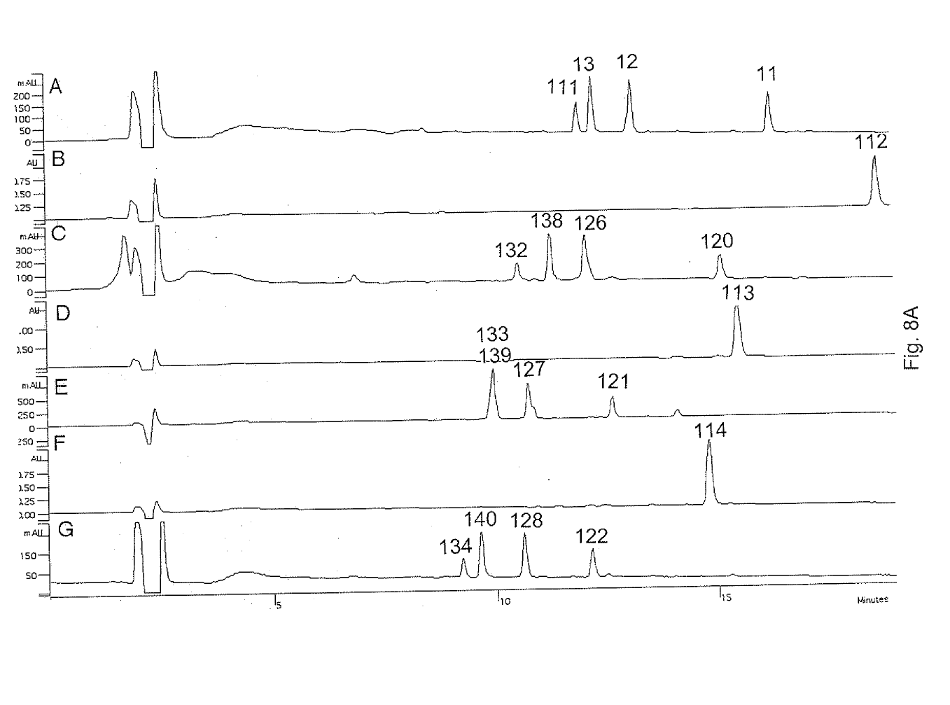

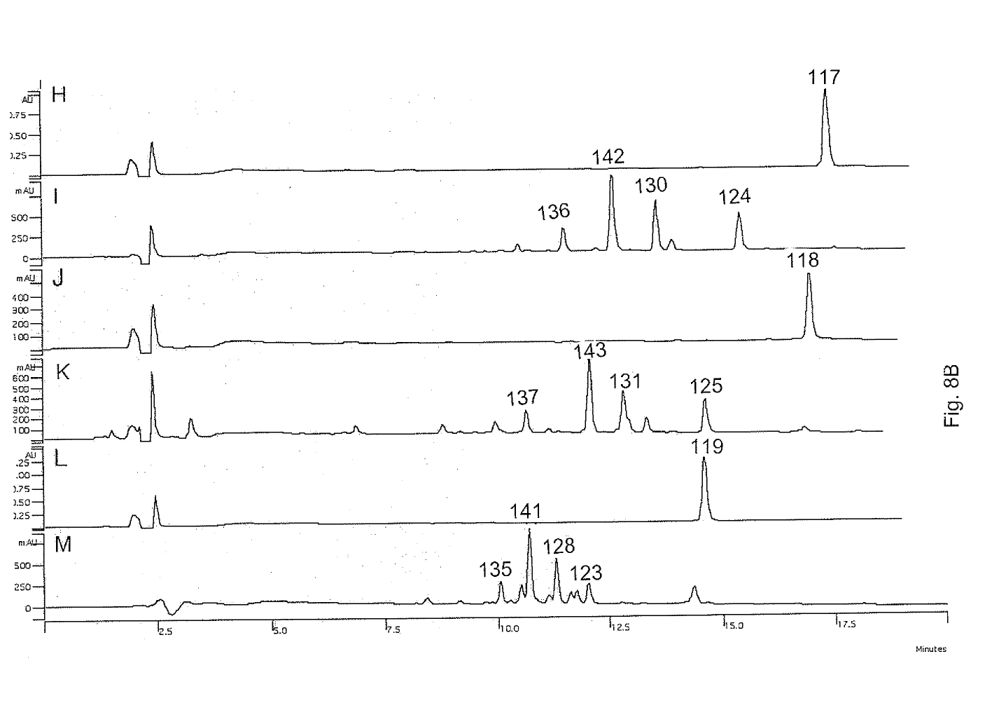

H₂O-mediated rearrangement of 112-114 and 117-119. A general reaction method is described as follows. Each of the purified 112-115 and 117-119 (15˜50 mg) was dissolved in appropriate volume of CHCl₃, transferred into a round flask (250˜500 mL) and evaporated to dryness. Then warm Milli-Q H₂O (37° C.) was added to the flask to form an approximate 0.4 mM solution. The mixture was stirred by rotation in water bath at 37° C. for 2 hr (this led to >90% conversion rate) and then at 90˜100° C. for additional 20 min to complete the conversion. The resulting mixture was evaporated under reduced pressure to dryness and the residue was subjected to HPLC and LC-ESI-MS analyses. All the congeners tested were converted to essentially four products (see FIG. 8 and Table 6).

Purification of the reaction products was achieved by silica gel flash column chromatography, eluted with a CHCl₃/MeOH (9.8:0.2-8:2, containing 0.1% HOAc) gradient and subsequently via semi-preparative HPLC. For example, the reaction products of 117 (50 mg) was loaded on silica gel flash column (1.3×30 cm) and eluted with a CHCl₃/MeOH/HOAc (98:2:0.1-90:10:0.1) gradient to afford purified 124 (10 mg), 142 (15 mg), and a mixture containing 136 and 130 that was further separated by semi-preparative HPLC to yield 136 (7 mg) and 130 (12 mg). Similar procedure resulted in the purification of all the compounds listed in Table 6.

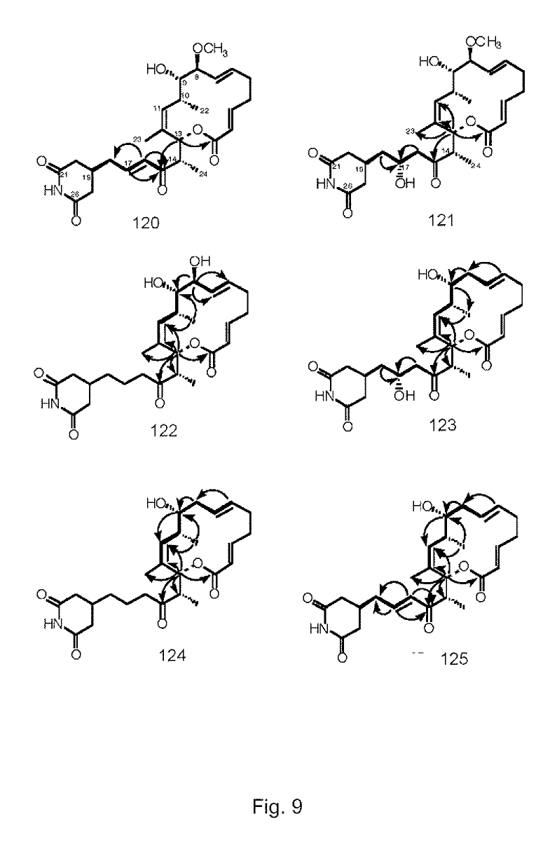

The structures of the products in Table 6 were deduced by comparison of their HPLC chromatographic properties with the corresponding rearrangement products of 110 that have been well established previously, and were further confirmed by ESI−MS data (Table 6). In addition, the structures of all the congeners (120-125) of 101 were further subjected to 1D and 2D NMR (COSY, TOCSY, HMQC and gHMBC) analyses (see FIG. 9 and Table 7-9). Representative dorrigocin congeners 127, 128, 130, 134, 136, 139, 140 and 142 were also confirmed by 1D and 2D NMR (COSY, HMQC and gHMBC) analyses (see Table 10-12).

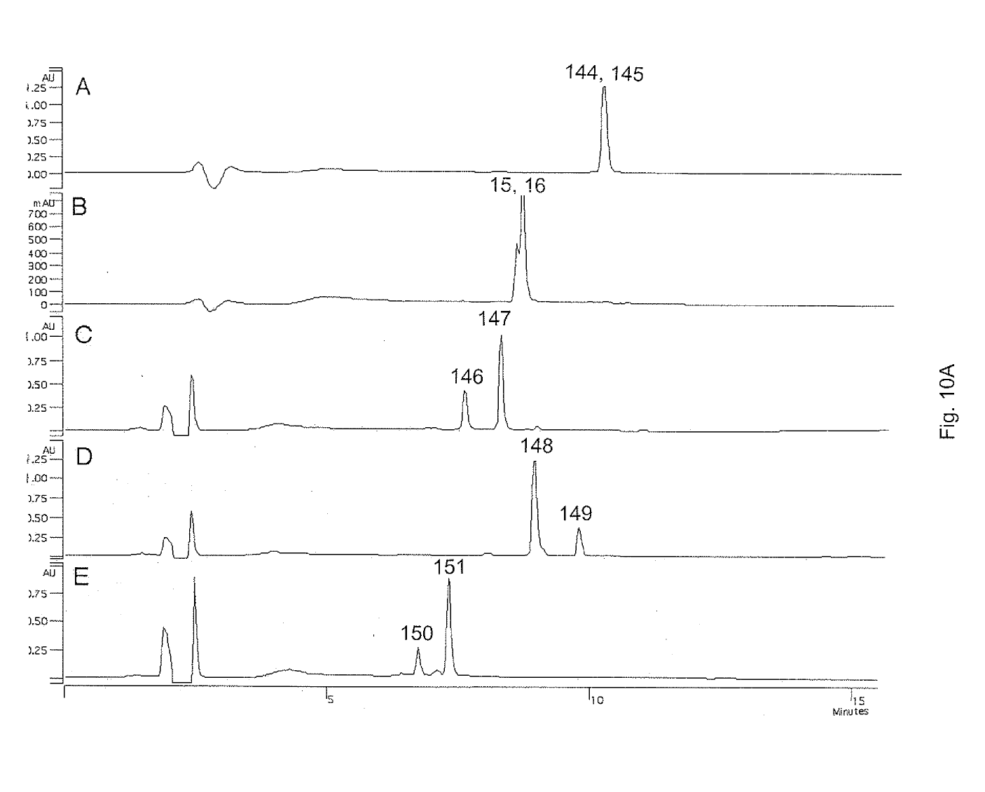

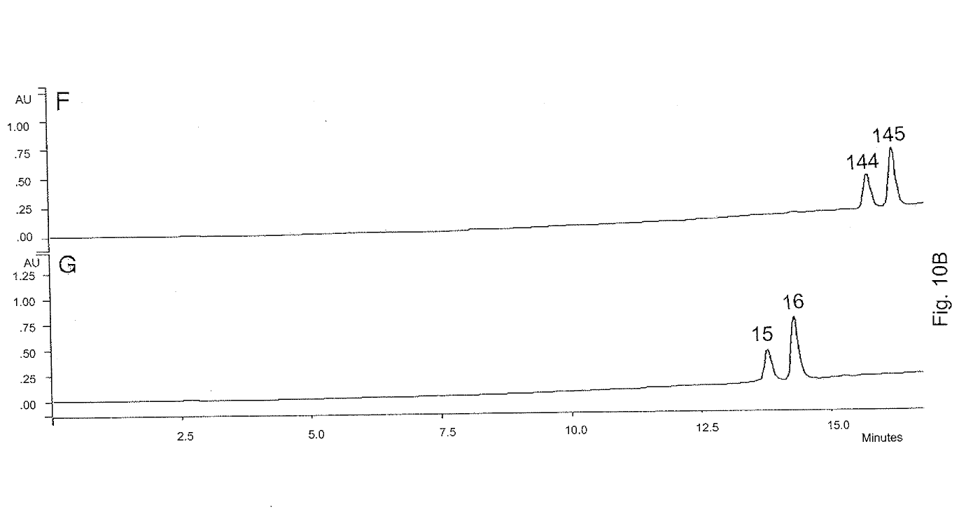

Cysteine 1,4-Michael addition of 110, 113, 114, 117 and 119: A general reaction approach is described as follows. To a solution of purified 110, 113, 114, 117, or 119 (10-30 mg) in MeOH ( 0.5 mM) at room temperature was slowly added an equal volume of solution of L-cysteine (1 mM) in H₂O. The resultant mixture was stirred at room temperature for 10 min. HPLC analysis revealed that the 1,4-Michael addition reaction was completed in 2 min. The reaction mixture was evaporated in vacuum to dryness. The residue was dissolved in small amount of MeOH-H₂O (1:1), filtrated, and analyzed by HPLC (see FIG. 10 and Table 13). Purification of the diasteromers (at C-3) was achieved by semi-preparative HPLC. While the two diasteromers of 44 and 45 (from 10) or 5 and 6 (from 13) were inseparable under the standard HPLC condition described above (i.e. a linear gradient of 15% CH₃CN to 80% CH₃CN in H₂O containing 0.1% HOAc over 20 min) described, they were well resolved by an alternative solvent system with a linear gradient of 20% MeOH to 80% MeOH in H₂O over 20 min (FIG. 10).

The structures of the cysteine adducts were confirmed by LC-ESI-MS (Table 13). Selected pairs of diasteromers, such as 105/106 and 144/145, were further verified by 1D and 2D NMR (COSY, HMQC and gHMBC) analyses (Tables 14-16). The ¹H and ¹³C NMR spectra data of 105 and 106 (all measured in D₂O) were in good agreement with those reported for NK30424 A and B, respectively.

Stereochemistry Determination.

Iso-Migrastatin (110) and its congeners (112-119). We have previously demonstrated that 110 undergoes rapid conversion to 101. Ju, J.; Lim, S.-K.; Jiang, H.; Shen, B. J. Am. Chem. Soc. 2005, 127, 1622-1623. Since the absolute stereochemistry of 101 has been determined by X-ray crystallographic analysis of an N-p-bromophenacylated derivative, we concluded that 110 has the same configuration as 101 at C-8, -9, -10, and -14 (i.e., 8S, 9S, 10S, and 14S for 110). Nakamura, N.; Takahashi, Y.; Naganawa, H.; Nakae, K.; Imoto, M.; Shiro, M.; Matsumura, K.; Watanabe, H.; Kitahara, T. J. Antibiot. 2002, 55, 442-444. This early work, however, left the stereogenic center at C-11 for 110 unassigned. The relative stereochemistry of 110 has recently been established by X-ray crystallographic analysis. Santi, D. V. Kosan Biosciences, Inc., Hayward, Calif., Determination of the relative stereochemistry of isomigrastation by X-ray crystallography. Taken together, these results have now allowed us finally complete the stereochemistry assignment for 110 with 11R configuration. Consequently, congeners 112-119 were assigned to have the same configuration at C-8, -9, -10, -11, and -14 (if applicable) as 110 on the basis of their biosynthetic origin (FIG. 7), which was further confirmed by comparative analyses of their ¹H and ¹³C NMR splitting patterns and chemical shifts. The C-16/C-17 trans double bond (for 112, 115, and 118) was based on its diagnostic coupling constant (J=16 Hz). The HO-group at C-17 (for 113 and 119) was assigned R configuration on the basis of its near identical splitting patterns and chemical shifts at C-15, -16, -17, -18, and -19 to those of streptimidone (108) (all measured in CDCl₃), the absolute R configuration of which had been determined and the high resolution NMR data of which had been reported previously. Allen, M. S.; Becker, A. M.; Rickards, R. W. Aust. J. Chem. 1976, 29, 673-679; Jeffs, P. W.; McWilliams, D. J. Am. Chem. Soc. 1981, 103, 6185-6192; Kim, B. S.; Moon, S. S.; Hwang, B. K. J. Agric. Food Chem. 1999, 47, 3372-3380 and references cited therein. The identical stereocenters at C-8, -9, -10, and -11 for 110 and its congeners (112-119) were consistent with the ¹H—¹H coupling constants [J_{H7,H8α}=4.0 Hz, J_{H8α,H9β}=9.0-9.5 Hz, J_{H9β,H8β}=3.5-4.0 Hz (if applicable), J_{H9β,H10β}=0 Hz, and J_{H11α,H10β}=3.5-4.0 Hz] and ¹³C NMR chemical as summarized in Tables 3-5.

Migrastatin (101) and its congeners (120-125). The absolute stereochemistry of 101 has been determined previously by X-ray crystallographic analysis. The absolute stereochemistry of 10 has been completely assigned in this work (see above). The stereochemistry relationship for 110 to undergo the H₂O-mediated rearrangement to 101 has been established previously. The same stereochemistry relationship between 110 and 101 was applied here to the stereochemistry assignments for congeners 120-125 according to their corresponding substrates 112, 113, 114, 119, 117, and 118, respectively. These assignments are in perfect agreement with their ¹H and ¹³C NMR data upon comparison with those of 101 as summarized in Tables 7-9.

Dorrigocin A (102) and 13-epi-dorrigocin A (111) and their congeners (126-137). The absolute stereochemistry of 102 and 111 has been determined, and the stereochemistry relationship for 110 to undergo the H₂O-mediated rearrangement to 102 and 111 has been established previously. The same stereochemistry relationship between 110 and its resultant products 102 and 111 was applied here to the stereochemistry assignments for congeners 126-137 according to their corresponding substrates 112, 113, 114, 119, 117, and 118, respectively. The chemical shifts of H-13 for 102 and its congeners resonate at ˜4.00 ppm (J=10.0 Hz), while those of H-13 for 111 and its congeners shift downfiled to 4.15 ppm (J=7.0 Hz). These assignments are in perfect agreement with selected ¹H and ¹³C NMR data upon comparison with 102 and 111, respectively, as summarized in Tables 10-12.

Dorrigocin B (103) and its congeners (138-143). We have previously assigned the stereochemistry for 103 as 8S, 9S, 10S, and 14S with the configuration at C-11 unassigned. Similar to those described for the complete stereochemistry assignment for 110 and its congeners 112-119, we can now complete the stereochemistry assignment for 103 with 11R configuration. The same stereochemistry relationship between 110 and 102 was applied here to the stereochemistry assignments for congeners 138-143 according to their corresponding substrates 112, 113, 114, 119, 117, and 118, respectively. These assignments are in perfect agreement with selected ¹H and ¹³C NMR data upon comparison with 103 as summarized in Tables 11 and 13.

NK30424 A (105) and B (106) and their congeners (144-151). First isolated from Streptomyces sp. NA30424, 105 and 106 were established as two stereoisomers at C-3, with their relative and absolute stereochemistry undetermined. Takayasu, Y.; Tsuchiya, K.; Aoyama, T.; Sukenaga, Y. J. Antibiot. 2001, 54, 1111-1115; Takayasu, Y.; Tsuchiya, K.; Su kenaga, Y. J. Antibiot. 2002, 55, 3 3 7-340. Viewing 105 and 106 as a cysteine 1,4-adduct at C-2/C-3 of 113, we incubated 113 with cysteine. It indeed afforded in quantitative yield two diastereomers at C-3 in an approximately 2:3 ratio (FIG. 10), whose identity as 105 and 106 was confirmed by their identical ¹H and ¹³C NMR data to those reported previously (Tables 15 and 16). Takayasu, Y.; Tsuchiya, K.; Aoyama, T.; Sukenaga, Y. J. Antibiot. 2001, 54, 1111-1115; Takayasu, Y.; Tsuchiya, K.; Sukenaga, Y. J. Antibiot. 2002, 55, 337-340. Since we have established the absolute stereochemistry of 113 (see above), the quantitative conversion of 113 to 105 and 106 upon cysteine addition allowed us to conclude that 105 and 106 have the same configuration as 113 at C-8, -9, -10, -11, -14, and -17 (i.e., 8S, 9S, 10S, 11R, 14S, and 17R). The same stereochemistry relationship between 113 and its resultant adducts 105 and 106 was applied here to the stereochemistry assignments for congeners 144-151 according to their corresponding substrates 110, 114, 117, and 119, respectively. These assignments are in perfect agreement with selected ¹H and ¹³C NMR data as summarized in Tables 14-16. Attempt to differentiate the two diastereomers at C-3 by NMR analysis was inconclusive, however, and the stereochemistry at C-3 for 105, 106 and their congeners 144-151, therefore, remained unassigned.

Table 3. ¹H NMR (500 MHz) spectral data of 110 and 112-114^{a} in CDCl₃.

TABLE 3

| Position | 110 | 112 | 113 | 114 |
|---|---|---|---|---|
| 2 | 5.69, 1H, d(16.0) | 5.69, 1H, d(15.5) | 5.70, 1H, d(16.0) | 5.70, 1H, d(15.5) |
| 3 | 6.65, 1H, ddd(16.0, 8.5, 7.5) | 6.64, 1H, ddd(15.5, 8.5, 7.5) | 6.66, 1H, ddd(16.0, 9.0, 7.0) | 6.65, 1H, ddd(15.5, 9.5, 7.5) |
| 4 | 2.15, 1H, m | 2.16, 1H, m | 2.18, 1H, m | 2.15, 1H, m |
| | 2.45, 1H, m | 2.45, 1H, m | 2.42, 1H, m | 2.43, 1H, m |
| 5 | 1.96, 1H, m | 1.96, 1H, m | 1.95, 1H, m | 1.95, 1H, m |
| | 2.60, 1H, m | 2.63, 1H, m | 2.62, 1H, m | 2.60, 1H, m |
| 6 | 5.60, 1H, ddd(15.5, 11.0, | 5.61, 1H, ddd(15.5, 11.0, | 5.61, 1H, ddd(15.5, 10.0, | 5.75, 1H, ddd(15.5, 9.5, 3.5) |
| | 4.5) | 4.0) | 4.0) | |
| 7 | 5.10, 1H, dd(15.5, 3.5) | 5.10, 1H, dd(15.5, 3.5) | 5.10, 1H, dd(15.5, 3.6) | 5.29, 1H, dd(15.5, 4.0) |
| 8 | 3.43, 1H, (overlap) | 3.43, 1H, m | 3.46, 1H, (overlap) | 3.96, 1H, dd(9.5, 4.0) |
| 9 | 3.75, 1H, d(9.5) | 3.77, 1H, d(9.0) | 3.76, 1H, d(9.0) | 3.94, 1H, d(9.0) |
| 10 | 1.85, 1H, m | 1.85, 1H, m | 1.86, 1H, m | 1.88, 1H, m |
| 11 | 5.20, 1H, (overlap) | 5.22, 1H, (overlap) | 5.22, 1H, d(4.0) | 5.21, 1H, d(4.0) |
| 13 | 5.20, 1H, (overlap) | 5.22, 1H, (overlap) | 5.17, 1H, br.d(9.5) | 5.24, 1H, br.d(10.0) |
| 14 | 3.43, 1H, m | 3.60, 1H, m | 3.46, 1H, (overlap) | 3.46, 1H, m |
| 16 | 2.39, 1H, m | 6.32, 1H, d(16.0) | 2.55, 1H, dd(18.0, 3.5); | 2.40, 1H, m |
| | 2.62, 1H, m | | 2.75, 1H, m | 2.62, 1H, m |
| 17 | 1.59, 2H, m | 6.76, 1H, dt(16.0, 7.0) | 4.08, 1H, m | 1.59, 2H, m |
| 18 | 1.36, 2H, m | 2.32, 2H, m | 1.58, 1H, ddd(14.0, 10.0, | 1.38, 2H, m |
| | | | 5.0) | |
| | | | 1.40, 1H, ddd(14.0, 9.0, 3.0) | |
| 19 | 2.10, 1H, m | 2.34, 1H, m | 2.46, 1H, m | 2.13, 1H, m |
| 20 | 2.25, 1H, m | 2.28, 1H, m | 2.32, 1H, m | 2.22, 1H, m |
| | 2.70, 1H, m | 2.73, 1H, m | 2.80, 1H, m | 2.68, 1H, m |
| 22 | 0.87, 3H, d(7.5) | 0.83, d(7.0) | 0.87, 3H, d(7.5) | 0.88, 3H, d(7.0) |
| 23 | 1.90, 3H, d(1.5) | 1.94, 3H, d(1.0) | 1.90, 3H, d(1.5) | 1.89, 3H, d(1.5) |
| 24 | 1.14, 3H, d(7.0) | 1.18, 3H, d(6.5) | 1.16, 3H, d(7.0) | 1.18, 3H, d(7.0) |
| 25 | 2.25, 1H, m | 2.28, 1H, m | 2.32, 1H, m | 2.22, 1H, m |
| | 2.70, 1H, m | 2.73, 1H, m | 2.75, 1H, m | 2.68, 1H, m |
| 8-OCH3 | 3.34, 3H, s | 3.34, 3H, s | 3.34, 3H, s | |
| NH | | 7.80, 1H, br.s | 7.86, 1H, br.s | 8.35, 1H, br.s |

^{a}Signals were assigned with aid of ¹H-¹H COSY, TOCSY, HMQC, and gHMBC experiments.

Table 4. ¹H NMR (500 MHz) spectral data of 115^{a} and 117-119^{a} in CDCl₃.

TABLE 4

| Position | 115 | 117 | 118 | 119 |
|---|---|---|---|---|
| 2 | 5.71, 1H, d(15.5) | 5.67, 1H, d(16.0) | 5.70, 1H, d(16.0) | 5.65, 1H, d(15.5) |
| 3 | 6.64, 1H, overlap | 6.62, 1H, ddd(16.0, 9.5, 7.5) | 6.64, 1H, ddd(16.0, 8.0, 8.0) | 6.61, 1H, ddd(15.5, 9.5, 7.5) |
| 4 | 2.16, 1H, m | 2.14, 1H, m | 2.12, 1H, m | 2.15, 1H, m |
| | 2.42, 1H, m | 2.42, 1H, m | 2.42, 1H, m | 2.42, 1H, m |
| 5 | 1.94, 1H, m | 1.88, 1H, m | 1.88, 1H, m | 1.90, 1H, m |
| | 2.60, 1H, m | 2.52, 1H, m | 2.52, 1H, m | 2.55, 1H, m |
| 6 | 5.76, 1H, (overlap) | 5.33, 1H, (overlap) | 5.36, 1H, ddd(15.5, 10.5, | 5.32, 1H, ddd(15.5, 10.5, |
| | | | 4.0) | 5.0) |
| 7 | 5.28, 1H, (overlap) | 5.02, 1H, ddd(15.5, 9.5, 6.0) | 5.04, 1H, ddd(15.5, 7.0, 6.5) | 5.00, 1H, ddd(15.5, 9.5, 6.0) |
| 8 | 3.97, 1H, dd(9.0, 4.0) | 2.10, 1H, m | 2.08, 1H, m | 2.15, 1H, m |
| | | 2.21, 1H, m | 2.22, 1H, m | 2.21, 1H, m |
| 9 | 3.77, 1H, d(9.5) | 4.01, 1H, dd(11.0, 4.0) | 4.02, 1H, dd(11.0, 3.5) | 3.97, 1H, dd(11.0, 4.0) |
| 10 | 1.90, 1H, m | 1.91, 1H, m | 1.92, 1H, m | 1.89, 1H, m |
| 11 | 5.24, 1H, (overlap) | 5.22, 1H, d(3.5) | 5.24, 1H, d(4.0) | 5.20, 1H, (overlap) |
| 13 | 5.25, 1H, (overlap) | 5.34, 1H, (overlap) | 5.32, 1H, d(9.5) | 5.20, 1H, (overlap) |
| 14 | 3.60, 1H, m | 3.43, 1H, m | 3.60, 1H, m | 3.42, 1H, m |
| 16 | 6.31, 1H, d(15.5) | 2.44, 1H, m | 6.26, 1H, d(15.0) | 2.50, 1H, m |
| | | 2.54, 1H, m | | 2.70, 1H, m |
| 17 | 6.79, 1H, dt(15.5, 7.0) | 1.60, 2H, m | 6.78, 1H, dt(15.0, 6.5) | 4.09, 1H, tt(9.5, 3.0) |
| 18 | 2.30, 2H, m | 1.34, 2H, m | 2.30, 2H, m | 1.55, 1H, ddd(14.0, 10.5, |
| | | | | 5.5) |
| | | | | 1.32, 1H, ddd(14.0, 8.5, 3.0) |
| 19 | 2.34, 1H, m | 1.90, 1H, m | 2.32, 1H, m | 2.45, 1H, m |
| 20 | 2.32, 1H, m | 2.26, 1H, m | 2.32, 1H, m | 2.31, 1H, m |
| | 2.76, 1H, m | 2.69, 1H, dd(17.0, 4.0) | 2.72, 1H, m | 2.76, 1H, m |
| 22 | 0.86, 3H, d(7.0) | 0.88, 3H, d(7.5) | 0.87, d(6.5) | 0.83, 3H, d(7.5) |
| 23 | 1.92, 3H, d(1.0) | 1.85, 3H, d(1.5) | 1.91, 3H, br.s | 1.85, 3H, d(1.0) |
| 24 | 1.20, 3H, d(7.0) | 1.17, 3H, d(7.0) | 1.22, 3H, d(7.0) | 1.16, 3H, d(7.0) |
| 25 | 2.30, 1H, m | 2.26, 1H, m | 2.32, 1H, m | 2.31, 1H, m |
| | 2.76, 1H, m | 2.69, 1H, dd(17.0, 4.0) | 2.72, 1H, m | 2.78, 1H, m |
| NH | 7.80, 1H, br.s | 8.39, 1H, br.s | 7.86, 1H, br.s | 7.95, 1H, br.s |

^{a}Signals were assigned with aid of ¹H-¹H COSY, TOCSY, HMQC, and gHMBC experiments.

Table 5. ¹³C NMR (125 MHz) spectral data of 110 and 112-119^{a} in CDCl₃.

TABLE 5

| Position | 110 | 112 | 113 | 114 | 115 | 117 | 118 | 119 |
|---|---|---|---|---|---|---|---|---|
| 1 | 167.8 | 167.0 | 167.8 | 167.7 | 167.8 | 167.8 | 168.0 | 167.9 |
| 2 | 125.2 | 125.3 | 125.1 | 125.2 | 125.2 | 125.1 | 125.3 | 125.1 |
| 3 | 150.8 | 150.6 | 151.0 | 151.0 | 151.0 | 150.9 | 150.8 | 151.0 |
| 4 | 30.3* | 30.3 | 30.3 | 30.4 | 30.4 | 30.6 | 30.6 | 30.6 |
| 5 | 32.9 | 32.9 | 33.0 | 32.8 | 32.8 | 32.9 | 33.0 | 33.0 |
| 6 | 129.3 | 129.3 | 129.5 | 128.3 | 128.3 | 131.4 | 131.2 | 130.7 |
| 7 | 130.5 | 130.6 | 130.4 | 134.5 | 134.8 | 130.8 | 130.9 | 131.5 |
| 8 | 81.8 | 81.9 | 81.7 | 71.7 | 71.8 | 38.0 | 38.2 | 38.2 |
| 9 | 73.4 | 73.4 | 73.7 | 74.6 | 74.8 | 71.4 | 71.5 | 71.7 |
| 10 | 38.3 | 38.3 | 38.4 | 38.6 | 38.7 | 39.7 | 39.7 | 39.8 |
| 11 | 82.4 | 82.5 | 82.2 | 81.7 | 81.9 | 81.5 | 81.9 | 81.5 |
| 12 | 134.3 | 134.5 | 135.3 | 134.2 | 134.3 | 134.9 | 135.1 | 135.8 |
| 13 | 128.2 | 127.9 | 127.3 | 127.9 | 127.8 | 127.4 | 127.5 | 126.6 |
| 14 | 46.2 | 44.8 | 46.8 | 46.1 | 44.9 | 46.0 | 44.8 | 46.6 |
| 15 | 211.0 | 199.7 | 212.3 | 210.9 | 199.6 | 210.9 | 199.6 | 212.2 |
| 16 | 40.1 | 131.6 | 47.5 | 40.2 | 131.3 | 40.2 | 131.4 | 47.6 |
| 17 | 20.5 | 141.2 | 65.3 | 20.4 | 141.8 | 20.5 | 141.8 | 65.0 |
| 18 | 34.4 | 37.7 | 41.2 | 34.2 | 37.6* | 34.2 | 37.5 | 40.8 |
| 19 | 30.3* | 29.8 | 27.3 | 30.2 | 29.7 | 30.4 | 29.6 | 27.2 |
| 20 | 37.9 | 37.5 | 38.7 | 38.0 | 37.5* | 37.8* | 37.4 | 38.6 |
| 21 | 172.3 | 171.6 | 172.3 | 172.4* | 171.7* | 172.6* | 171.7 | 172.4 |
| 22 | 10.7 | 10.7 | 10.8 | 10.7 | 10.8 | 9.4 | 9.5 | 9.4 |
| 23 | 13.5 | 13.3 | 13.7 | 14.2 | 14.0 | 14.6 | 14.2 | 14.5 |
| 24 | 15.9 | 15.5 | 15.7 | 16.2 | 16.0 | 16.4 | 16.2 | 15.9 |
| 25 | 37.9 | 37.5 | 37.5 | 37.9 | 37.4* | 37.8* | 37.3 | 37.4 |
| 26 | 172.3 | 171.6 | 172.2 | 172.4* | 171.7* | 172.6* | 171.7 | 172.4 |
| 8- | 57.3 | 57.3 | 57.3 | | | | | |
| OCH3 | | | | | | | | |

^{a}Signals were assigned with aid of ¹H—¹H COSY, TOCSY, HMQC, and gHMBC experiments.
*Overlapped signals or signals that could be interchangeable.

Table 6. Dorrigocin (126-143) and migrastatin (120-125) analogs resulted from H₂O-mediated rearrangement of 112-114 and 117-119 and their LC-ESI-MS data (acquired in both positive and negative mode)

TABLE 6

| | Products | | | |
|---|---|---|---|---|
| Substrates | 13-epi-dorrigocin A series | dorrigocin B-series | Dorrigocin A series | Migrastatin-series |
| 112 | 132 | 138 | 126 | 120 |
| | m/z527.9([M+Na]⁺); | m/z527.9([M+Na]⁺); | m/z527.9([M+Na]⁺); | m/z510.0([M+Na]⁺); |
| | m/z504.1([M−H])⁻ | m/z504.1([M−H])⁻ | m/z504.1([M−H])⁻ | m/z486.1([M−H])⁻ |
| 113 | 133 | 139 | 127 | 121 |
| | m/z545.9([M+Na]⁺); | m/z546.0([M+Na]⁺); | m/z545.9([M+Na]⁺); | m/z527.9([M+Na]⁺); |
| | m/z522.2([M−H])⁻ | m/Z522.2([M−H])⁻ | m/z522.2[M−H])⁻ | m/z504.2([M−H])⁻ |
| 114 | 134 | 140 | 128 | 122 |
| | m/z515.9([M+Na]⁺); | m/z515.9([M+Na]⁺); | m/z515.9([M+Na]⁺); | m/z476.0([M+H])⁺; |
| | m/z492.2([M−H])⁻ | m/z492.2([M−H])⁻ | m/z492.2([M−H])⁻ | m/z474.2([M−H])⁻ |
| 117 | 136 | 142 | 130 | 124 |
| | m/z500.0([M+Na]⁺); | m/z500.0([M+Na]⁺); | m/z500.0([M+Na]⁺); | m/z460.2([M+H])⁺; |
| | m/z476.3([M−H])⁻ | m/z476.3([M−H])⁻ | m/z476.3([M−H])⁻ | m/z458.3([M−H])⁻ |
| 118 | 137 | 143 | 131 | 125 |
| | m/z474.4([M−H])⁻ | m/z474.2([M−H])⁻ | m/z474.2([M−H])⁻ | m/z456.2([M−H])⁻ |
| 119 | 135 | 141 | 129 | 123 |
| | m/z515.9([M+H])⁺; | m/z516.0([M+H])⁺; | m/z516.0([M+H])⁺; | m/z474.2([M−H])⁻ |
| | m/z492.2([M−H])⁻ | m/z492.2([M−H])⁻ | m/z492.2([M−H])⁻ | |

Table 7. ¹H NMR (500 MHz) spectral data of 101 and 120-122^{a} in CDCl₃.

TABLE 7

| Position | 101 | 120 | 121 | 122 |
|---|---|---|---|---|
| 2 | 5.55, 1H, dd(16.0, 1.5) | 5.59, 1H, d(16.0) | 5.59, 1H, d(15.5) | 5.56, 1H, dd(16.0, 1.5) |
| 3 | 6.47, 1H, ddd(16.0, 10.0, | 6.50, 1H, ddd(16.0, 10.0, | 6.51, 1H, ddd(15.5, 10.5, | 6.47, 1H, ddd(16.0, 10.0, |
| | 3.5) | 3.5) | 4.0) | 3.0) |
| 4 | 2.20, 1H, m | 2.14, 1H, m | 2.22, 1H, m | 2.17, 1H, m |
| | 2.41, 1H, m | 2.42, 1H, m | 2.44, 1H, m | 2.44, 1H, m |
| 5 | 2.20, 1H, m | 2.18 1H, m | 2.24, 1H, m | 2.17, 1H, m |
| | 2.41, 1H, m | 2.42, 1H, m | 2.42, 1H, m | 2.44, 1H, m |
| 6 | 5.50, 1H, ddd(15.5, | 5.53, 1H, ddd(15.5, 9.0, 5.0) | 5.54, 1H, ddd(16.0, 9.0, 4.5) | 5.63, 1H, m |
| | 10.0, 5.0) | | | |
| 7 | 5.22, 1H, dd(15.5, 5.0) | 5.23, 1H, dd(15.5, 5.0) | 5.25, 1H, dd(16.0, 4.5) | 5.44, 1H, dd(15.5, 4.0) |
| 8 | 3.46, 1H, dd(8.5, 5.0) | 3.48, 1H, dd(8.0, 5.0) | 3.48, 1H, dd(8.5, 5.0) | 4.02, 1H, dd(, 4.5) |
| 9 | 3.02, 1H, dd(8.5, 1.5) | 3.08, 1H, br.d(8.5) | 3.05, 1H, br.d(8.5) | 2.97*, 1H, m |
| 10 | 2.91, 1H, m | 2.97, 1H, m | 2.94, 1H, m | 2.97*, 1H, m |
| 11 | 5.62, 1H, dd(10.5, 1.5) | 5.66, 1H, d(10.5) | 5.68, 1H, d(11.0) | 5.55, 1H, dd(10.5, 1.0) |
| 13 | 5.06, 1H, d(10.0) | 5.22, 1H, d(9.5) | 5.10, 1H, d(10.0) | 5.04, 1H, d(10.0) |
| 14 | 2.95, 1H, dd(10.0, 8.5) | 3.10, 1H, m | 2.96, 1H, m | 2.80, 1H, m |
| 16 | 2.48, 2H, t(7.0) | 6.35, 1H, d(15.5) | 2.57, 1H, dd(18.0, 8.5) | 2.50, 2H, m |
| | | | 2.66, 1H, dd(18.0, 2.0) | |
| 17 | 1.59, 2H, m | 6.82, 1H, dt(15.5, 7.0) | 4.12, 1H, t-like(9.0) | 1.62, 2H, m |
| 18 | 1.32, 2H, m | 2.30, 2H, m | 1.36, 1H, ddd(14.0, 9.0, 4.0) | 1.35, 2H, m |
| | | | 1.62, 1H, m | |
| 19 | 2.10, 1H, m | 2.32, 1H, m | 2.50, 1H, m | 2.14, 1H, m |
| 20 | 2.20, 1H, m | 2.34, 1H, m | 2.32, 1H, m | 2.26, 1H, m |
| | 2.66, 1H, m | 2.72, 1H, br.d(13.5) | 2.80, 1H, m | 2.69, 1H, m |
| 22 | 0.94, 3H, d(7.0) | 0.99, 3H, d(7.0) | 0.98, 3H, d(7.0) | 0.96, d(7.0) |
| 23 | 1.83, 3H, d(1.5) | 1.86, 3H, br.s | 1.87, 3H, br.s | 1.86, 3H, d(1.5) |
| 24 | 1.10, 3H, d(8.5) | 1.17, 3H, d(7.0) | 1.16, 3H, d(7.5) | 1.12, 3H, d(7.5) |
| 25 | 2.20, 1H, m | 2.34, 1H, m | 2.30, 1H, m | 2.26, 1H, m |
| | 2.66, 1H, m | 2.72, 1H, br.d(13.5) | 2.82, 1H, m | 2.69, 1H, m |
| 8-OCH₃ | 3.28, 3H, s | 3.32, 3H, s | 3.32, 3H, s | |
| NH | 8.40, br.s | 7.79, br.s | 7.80 br.s | 8.33 br.s |

^{a}Signals were assigned with aid of COSY, HMQC, TOCSY, and gHMBC experiments.
*Overlapped signals.

Table 8. ¹H NMR (500 MHz) spectral data of 123-125^{a} in CDCl₃.

TABLE 8

| Position | 123 | 124 | 125 |
|---|---|---|---|
| 2 | 5.56, 1H, dd(16.0, 2.0) | 5.56, 1H, dd(16.0, 1.5) | 5.56, 1H, dd(15.5, 1.5) |
| 3 | 6.49, 1H, ddd(16.0, 11.0, 3.5) | 6.47, 1H, ddd(16.0, 10.0, 3.0) | 6.46, 1H, ddd(16.0, 10.5, 3.5) |
| 4 | 2.12, 1H, m | 2.15, 1H, m | 2.10, 1H, m |
| | 2.46, 1H, m | 2.40, 1H, m | 2.42, 1H, m |
| 5 | 2.10, 1H, m | 2.15, 1H, m | 2.08 1H, m |
| | 2.38, 1H, m | 2.40, 1H, m | 2.34, 1H, m |
| 6 | 5.26*, 1H, m | 5.26*, 1H, m | 5.25*, 1H, m |
| 7 | 5.26*, 1H, m | 5.26*, 1H, m | 5.25*, 1H, m |
| 8 | 2.18, 2H, m | 2.19, 2H, m | 2.18, 2H, m |
| 9 | 3.29, 1H, td-like(8.5, 1.5) | 3.28, 1H, dd(8.5, 1.5) | 3.31, 1H, td-like(8.0, 1.5) |
| 10 | 2.94, 1H, m | 2.95, 1H, m | 2.99, 1H, m |
| 11 | 5.56, 1H, dd(11.5, 1.5) | 5.54, 1H, dd(10.0, 1.5) | 5.54, 1H, dd(11.0, 1.0) |
| 13 | 5.04, 1H, d(11.0) | 5.03, 1H, d(10.5) | 5.13, 1H, d(10.0) |
| 14 | 3.00, 1H, m | 3.00, 1H, dd(10.5, 7.0) | 3.15, 1H, m |
| 16 | 2.58, 1H, dd(18.0, 9.0) | 2.52, 2H, m | 6.35, 1H, d(15.5) |
| | 2.68, 1H, dd(18.0, 3.0) | | 6.82, 1H, dt(15.5, 7.0) |
| 17 | 4.13, 1H, t-like(9.5) | 1.63, 2H, m | 2.30, 2H, m |
| 18 | 1.37, 1H, ddd(14.0, 9.0, 3.0) | 1.36, 2H, m | 2.30, 2H, m |
| | 1.63, 1H, ddd(14.0, 10.5, 5.0) | | |
| 19 | 2.50, 1H, m | 2.10, 1H, m | 2.34, 1H, m |
| 20 | 2.34, 1H, m | 2.25, 1H, m | 2.34, 1H, m |
| | 2.76, 1H, m | 2.70, H, m | 2.72, 1H, m |
| 22 | 0.95, 3H, d(7.0) | 0.94, 3H, d(7.0) | 0.96, 3H, d(7.0) |
| 23 | 1.89, 3H, d(1.5) | 1.88, 3H, d(1.5) | 1.89, 3H, d(1.0) |
| 24 | 1.17, 3H, d(7.0) | 1.13, 3H, d(7.5) | 1.18, 3H, d(7.0) |
| 25 | 2.32, 1H, m | 2.25, 1H, m | 2.36, 1H, m |
| | 2.78, 1H, m | 2.70, 1H, m | 2.72, 1H, m |
| NH | 7.82 br.s | | 7.87 br.s |

^{a}Signals were assigned with aid of COSY, HMQC, TOCSY, and gHMBC experiments.
*Overlapped signals.

Table 9. ¹³C NMR (125 MHz) spectral data of 101 and 120-125^{a} in CDCl₃.

TABLE 9

| Position | 101 | 120 | 121 | 122 | 123 | 124 | 125 |
|---|---|---|---|---|---|---|---|
| 1 | 164.0 | 164.3 | 164.2 | 164.1 | 164.2 | 164.2 | 164.3 |
| 2 | 122.3 | 122.4 | 122.2 | 122.3 | 122.0 | 122.2 | 122.2 |
| 3 | 150.1 | 150.1 | 150.5 | 150.5 | 151.0 | 150.6 | 150.6 |
| 4 | 30.5 | 30.3 | 30.4 | 30.5* | 30.7 | 30.6* | 30.6 |
| 5 | 31.2 | 31.4 | 31.4 | 31.1 | 31.3 | 31.2 | 31.3 |
| 6 | 130.6 | 130.9 | 130.9 | 129.0 | 132.0 | 132.1 | 132.1 |
| 7 | 128.1 | 128.3 | 128.3 | 131.4 | 128.4 | 127.6 | 128.4 |
| 8 | 82.7 | 82.8 | 82.7 | 72.3 | 38.1 | 38.1 | 38.1 |
| 9 | 78.0 | 78.1 | 78.2 | 79.7 | 76.2 | 76.2 | 76.2 |
| 10 | 32.1 | 32.3 | 32.3 | 32.7 | 33.2 | 33.2 | 33.2 |
| 11 | 133.2 | 133.4 | 133.7 | 132.9 | 133.6 | 133.3 | 133.3 |
| 12 | 131.3 | 131.3 | 131.2 | 131.9 | 132.2 | 132.2 | 132.2 |
| 13 | 77.1 | 76.9 | 77.2^{b} | 77.3 | 77.2^{b} | 77.0 | 77.2^{b} |
| 14 | 51.2 | 49.9 | 52.0 | 51.3 | 51.9 | 51.4 | 49.7 |
| 15 | 211.0 | 199.8 | 213.1 | 211.0 | 213.1 | 211.1 | 199.9 |
| 16 | 40.3 | 130.5 | 47.6 | 40.4 | 47.8 | 40.4 | 130.6 |
| 17 | 20.3 | 142.3 | 65.0 | 20.3 | 65.0 | 20.4 | 142.3 |
| 18 | 34.3 | 37.6* | 40.9 | 34.3 | 40.9 | 34.4 | 37.6* |
| 19 | 30.2 | 30.0 | 27.4 | 30.5* | 27.3 | 30.6* | 29.9 |
| 20 | 37.8* | 37.6* | 38.7 | 37.9 | 38.7 | 37.9 | 37.6* |
| 21 | 172.5 | 171.1 | 172.1 | 172.4 | 172.1 | 172.1* | 171.2* |
| 22 | 13.5* | 13.6 | 13.6 | 13.3 | 11.9 | 11.9 | 11.9 |
| 23 | 26.1 | 26.0 | 26.3 | 26.2 | 26.5 | 26.4 | 26.3 |
| 24 | 13.5* | 13.4 | 13.5 | 13.6 | 13.6 | 13.6 | 13.6 |
| 25 | 37.8* | 37.5* | 37.4 | 37.9 | 37.4 | 37.9 | 37.5* |
| 26 | 172.5 | 171.1 | 172.0 | 172.4 | 171.9 | 172.1* | 171.2* |
| 8-OCH₃ | 57.0 | 57.1 | 57.2 | | | | |

^{a}Signals were assigned with aid of COSY, HMQC, TOCSY, and gHMBC experiments.
^{b}Signals were overlapped with solvent peak but can be resolved by HMQC experiment.
*Overlapped signals.

Table 10. ¹H NMR (500 MHz) spectral data of 127, 128, 130, and 134 in MeOH-d₄.

TABLE 10

| Position | 127^{a} | 128^{a} | 130^{a} | 134^{a} |
|---|---|---|---|---|
| 2 | 5.85, 1H, d(15.5) | 5.84, 1H, d(15.5) | 5.85, 1H, d(15.5) | 5.84, 1H, d(15.0) |
| 3 | 6.88, 1H, dt(15.5, 7.0) | 6.86, 1H, dt(15.5, 7.0) | 6.88, 1H, dt(15.5, 7.0) | 6.87, 1H, dt(15.5, 6.5) |
| 4 | 2.36, 2H, m | 2.31, 2H, m | 2.30, 2H, m | 2.34, 2H, m |
| 5 | 2.24, 2H, m | 2.24, 2H, m | 2.17, 2H, m | 2.30, 2H, m |
| 6 | 5.72, 1H, dt(15.5, 6.0) | 5.72, 1H, dt(15.5, 6.5) | 5.53*, 1H, m | 5.74, 1H, dt(15.5, 6.0) |
| 7 | 5.50, 1H, dd(15.5, 8.0) | 5.62, 1H, dd(15.5, 7.0) | 5.53*, 1H, m | 5.61, 1H, dd(15.5, 7.0) |
| 8 | 3.55, 1H, dd(8.5, 4.0) | 4.00*, 1H, m | 2.00, 1H, m | 3.87, 1H, dd(6.5, 3.5) |
| | | | 2.30, 1H, m | |
| 9 | 3.19, 1H, dd(9.0, 4.0) | 3.20, 1H, dd(7.0, 4.0) | 3.35, 1H, td(8.0, 4.0) | 3.11, 1H, dd(7.5, 3.5) |
| 10 | 2.74, 1H, m | 2.64, 1H, m | 2.46, 1H, m | 2.70, 1H, m |
| 11 | 5.30, 1H, d(11.0) | 5.31, 1H, d(8.5) | 5.27, 1H, d(11.0) | 5.25, 1H, d(10.0) |
| 13 | 4.03, 1H, d(10.0) | 4.00*, 1H, m | 4.01, 1H, d(10.0) | 4.15, 1H, d(7.0) |
| 14 | 2.80, 1H, m | 2.77, 1H, m | 2.77, 1H, m | 2.82, 1H, quintet(7.0) |
| 16 | 1.52, 2H, m | 2.63, 2H, m | 2.65, 2H, m | 2.55, 2H, m |
| 17 | 4.18, 1H, m | 1.61, 2H, m | 1.61, 2H, m | 1.56, 2H, m |
| 18 | 2.74, 1H, m | 1.38, 2H, m | 1.38, 2H, m | 1.38, 2H, m |
| | 2.82, 1H, m | | | |
| 19 | 2.40, 1H, m | 2.13, 1H, m | 2.18, 1H, m | 2.12, 1H, m |
| 20 | 2.38, 1H, m | 2.32, 1H, m | 2.32, 1H, m | 2.30, 1H, m |
| | 2.78, 1H, m | 2.64, 1H, m | 2.68, 1H, m | 2.68, 1H, m |
| 22 | 0.98, 3H, d(7.0) | 0.99, 3H, d(7.0) | 0.98, 3H, d(7.0) | 0.99, 3H, d(6.5) |
| 23 | 1.63, 3H, d(1.5) | 1.64, 3H, d(1.0) | 1.62, 3H, d(1.5) | 1.65, 3H, d(1.0) |
| 24 | 0.85, 3H, d(6.5) | 0.82, 3H, d(7.0) | 0.82, 3H, d(7.0) | 1.07, 3H, d(7.0) |
| 25 | 2.40, 1H, m | 2.32, 1H, m | 2.32, 1H, m | 2.30, 1H, m |
| | 2.70, 1H, m | 2.64, 1H, m | 2.68, 1H, m | 2.68, 1H, m |
| 8-OCH₃ | 3.20, 3H, s | | | |

^{a}Signals were assigned with aid of COSY, HMQC and gHMBC experiments.
*Overlapped signals.

Table 11. ¹H NMR (500 MHz) spectral data of 136, 139, 140, and 142 in MeOH-d₄.

TABLE 11

| Position | 136^{a} | 139^{a} | 140^{a} | 142^{a} |
|---|---|---|---|---|
| 2 | 5.85, 1H, d(16.0) | 5.85, 1H, d(15.5) | 5.84, 1H, d(15.5) | 5.81, 1H, d(15.5) |
| 3 | 6.80, 1H, dt(16.0, 6.5) | 6.92, 1H, dt(15.5, 7.0) | 6.82, 1H, dt(15.5, 6.5) | 6.94, 1H, dt(15.5, 7.0) |
| 4 | 2.20, 2H, m | 2.36, 2H, m | 2.32, 2H, m | 2.32, 2H, m |
| 5 | 2.28, 2H, m | 2.30, 2H, m | 2.26, 2H, m | 2.20, 2H, m |
| 6 | 5.52*, 1H, m | 5.79, 1H, dt(15.5, 7.0) | 5.76, 1H, dt(15.5, 6.5) | 5.50*, 1H, m |
| 7 | 5.52*, 1H, m | 5.25, 1H, dd(15.5, 7.0) | 5.46, 1H, dd(15.5, 8.0) | 5.50*, 1H, m |
| 8 | 1.98, 1H, m | 3.50*, 1H, m | 3.98*, 1H, m | 2.25, 2H, m |
| | 2.22, 1H, m | | | |
| 9 | 3.27, 1H, td(8.0, 3.5) | 3.46, 1H, dd(8.5, 2.0) | 3.30^{b}, 1H, m | 3.59, 1H, td(8.0, 3.0) |
| 10 | 2.42, 1H, m | 1.75, 1H, m | 1.79, 1H, dt(7.0, 2.5) | 1.70, 1H, m |
| 11 | 5.23, 1H, d(10.0) | 3.94, 1H, d(8.5) | 3.98*, 1H, m | 4.03, 1H, d(6.5) |
| 13 | 4.16, 1H, d(7.5) | 5.25, 1H, d(8.5) | 5.31, 1H, d(10.0) | 5.32, 1H, d(9.5) |
| 14 | 2.81, 1H, quintet(7.0) | 3.50*, 1H, m | 3.52, 1H, m | 3.52, 1H, m |
| 16 | 2.57, 2H, m | 1.60, 2H, m | 2.52, 2H, m | 2.52, 2H, m |
| 17 | 1.55, 2H, m | 4.14, 1H, m | 1.58, 2H, m | 1.57, 2H, m |
| 18 | 1.35, 2H, m | 2.49, 1H, dd(17.5, 4.0) | 1.37, 2H, m | 1.35, 2H, m |
| | | 2.70, 1H, m | | |
| 19 | 2.13, 1H, m | 2.38, 1H, m | 2.16, 1H, m | 2.20, 1H, m |
| 20 | 2.32, 1H, m | 2.36, 1H, m | 2.34, 1H, m | 2.32, 1H, m |
| | 2.66, 1H, m | 2.78, 1H, m | 2.64, 1H, dd(17.0, 4.5) | 2.64, 1H, m |
| 22 | 0.99, 3H, d(7.0) | 0.94, 3H, d(7.0) | 0.92, 3H, d(7.0) | 0.89, 3H, d(7.0) |
| 23 | 1.63, 3H, d(1.0) | 1.68, 3H, d(1.5) | 1.67, 3H, d(1.0) | 1.65, 3H, d(1.5) |
| 24 | 1.07, 3H, d(7.0) | 1.11, 3H, d(6.5) | 1.12, 3H, d(6.5) | 1.12, 3H, d(6.5) |
| 25 | 2.32, 1H, m | 2.38, 1H, m | 2.34, 1H, m | 2.32, 1H, m |
| | 2.66, 1H, m | 2.70, 1H, m | 2.64, 1H, dd(17.0, 4.5) | 2.64, 1H, m |
| 8-OCH₃ | | 3.30, 3H, s | | |

^{a}Signals were assigned with aid of COSY, HMQC and gHMBC experiments.
*Overlapped signals.
^{b}Overlapped with solvent peak.

Table 12 ¹³C NMR (125 MHz) spectral data of 128, 130, 134, 136, 139, 140, and 142 in MeOH-d₄.

TABLE 12

| position | 128^{a} | 130^{a} | 134^{a} | 136^{a} | 139^{a} | 140^{a} | 142^{a} |
|---|---|---|---|---|---|---|---|
| 1 | # | # | # | # | # | 168.0^{b} | 170.2 |
| 2 | 123.6^{b} | # | 123.8^{b} | # | # | 124.6^{b} | 123.1 |
| 3 | 147.4 | 148.0^{b} | 147.0^{b} | 146.6^{b} | 149.4^{b} | 146.4^{b} | 150.6 |
| 4 | 32.9 | 33.2 | 32.9 | 32.7 | 32.8 | 32.8 | 33.2 |
| 5 | 32.3 | 32.6 | 32.3 | 33.2 | 32.1 | 32.3 | 32.4 |
| 6 | 132.3 | 132.4 | 132.1 | 132.7 | 136.6 | 133.7 | 132.7 |
| 7 | 133.3 | 129.5 | 133.6 | 129.4 | 129.1 | 132.2 | 129.1 |
| 8 | 74.5 | 39.2 | 74.3 | 39.5 | 86.4 | 75.5 | 39.5 |
| 9 | 79.3 | 76.7 | 79.6 | 76.9 | 75.5 | 77.1 | 74.3 |
| 10 | 36.0 | 39.3 | 36.0 | 39.6 | 38.6 | 38.5 | 40.3 |
| 11 | 133.8 | 133.6 | 131.7 | 131.7 | 81.1 | 80.4 | 80.3 |
| 12 | 136.0 | 136.1 | 136.5 | 136.3 | 140.6 | 140.4 | 140.6 |
| 13 | 82.1 | 82.1 | 79.0 | 78.8 | 128.0 | 127.7 | 127.0 |
| 14 | 50.4 | 50.4 | 51.3 | 51.3 | 47.8 | 47.3 | 47.2 |
| 15 | 216.8 | 216.8 | 215.0 | 215.1 | 211.0 | 214.1 | 214.1 |
| 16 | 43.8 | 43.8 | 42.7 | 42.6 | 42.8 | 41.5 | 41.4 |
| 17 | 21.2 | 21.3 | 21.5 | 21.4 | 66.0 | 21.7 | 21.7 |
| 18 | 35.4 | 35.4 | 35.4 | 35.4 | 38.6 | 95.4 | 35.5 |
| 19 | 31.7 | 31.7 | 31.8 | 31.8 | 28.8 | 31.7 | 31.7 |
| 20 | 38.7* | 38.7* | 38.7* | 38.7* | 39.4 | 38.6* | 38.7* |
| 21 | 175.8* | 175.7* | 175.6* | 175.6* | 175.7 | 175.8* | 175.7* |
| 22 | 16.5 | 16.9 | 16.7 | 17.1 | 8.8 | 8.6 | 7.7 |
| 23 | 11.0 | 11.2 | 12.9 | 13.0 | 12.1 | 12.7 | 13.1 |
| 24 | 14.6 | 14.6 | 12.6 | 12.6 | 16.0 | 16.7 | 16.7 |
| 25 | 38.7* | 38.7* | 38.7* | 38.7* | 38.1 | 38.6* | 38.7* |
| 26 | 175.8* | 175.7* | 175.6* | 175.6* | 175.5 | 175.8* | 175.7* |
| 8-OCH₃ | | | | | 56.7 | | |

^{a}Signals were assigned with aid of COSY, HMQC and gHMBC experiments.
^{b}Signals were assigned on the basis of HMQC or gHMBC experiment only.
# not observed.
*Overlapped signals.

Table 13. Cysteine 1,4-Michael addition products of 110, 113, 114, 117, 119 and their LC-MS data (acquired in both positive and negative mode).

TABLE 13

| 110 or its congener | Products | |
|---|---|---|
| 110 | 144 | 145 |
| | m/z476.2([M+H])⁺; | m/z476.2([M+H])⁺; |
| | m/z476.2([M−H])⁻ | m/z476.2([M−H])⁻ |
| 113 | 105 | 106 |
| | m/z627.0([M+H])⁺; | m/z626.9([M+H])⁺; |
| | m/z625.3([M−H])⁻ | m/z625.3([M−H])⁻ |
| 114 | 146 | 147 |
| | m/z597.0([M+H])⁺; | m/z597.0([M+H])⁺; |
| | m/z595.2([M−H])⁻ | m/z595.2([M−H])⁻ |
| 117 | 148 | 149 |
| | m/z579.2([M+H])⁺; | m/z579.2([M+H])⁺; |
| | m/z581.0([M−H])⁻ | m/z581.0([M−H])⁻ |
| 119 | 150 | 151 |
| | m/z596.9([M+H])⁺; | m/z597.0([M+H])⁺; |
| | m/z595.2([M−H])⁻ | m/z595.2([M−H])⁻ |

Table 14: ¹H NMR (500 MHz) spectral data of compounds 144 and 145 in MeOH-d₄.

TABLE 14

| Position | 144^{a} | 145^{a} |
|---|---|---|
| 2 | 2.54, 1H, dd(18.0, 11.0) | 2.38*, 1H, m |
| | 2.77, 1H, dd(18.0, 3.0) | 2.95*, 1H, m |
| 3 | 3.40, 1H, m | 3.13, 1H, m |
| 4 | 1.90, 1H, m | 1.60, 1H, m |
| | 2.18, 1H, m | 1.82, 1H, m |
| 5 | 2.23, 2H, m | 2.24, 2H, m |
| 6 | 5.71, 1H, ddd(15.5, 10.0, 6.5) | 5.74, 1H, ddd(15.5, 10.0, 7.0) |
| 7 | 5.38, 1H, dd(15.5, 6.5) | 5.35, 1H, dd(15.5, 7.0) |
| 8 | 3.35*, 1H, m | 3.40, 1H, dd(9.0, 7.0) |
| 9 | 3.64, 1H, d(9.0) | 3.68, 1H, d(9.0) |
| 10 | 2.04, 1H, m | 2.14, 1H, m |
| 11 | 4.99, 1H, d(3.5) | 5.10, 1H, d(3.0) |
| 13 | 5.15, 1H, d(10.0) | 5.15, 1H, d(10.0) |
| 14 | 3.53, 1H, m | 3.53, 1H, m |
| 16 | 2.48, 1H, m | 2.48, 1H, m |
| | 2.68, 1H, m | 2.66, 1H, m |
| 17 | 1.56, 2H, m | 1.56, 2H, m |
| 18 | 1.34, 2H, m | 1.34, 2H, m |
| 19 | 2.20, 1H, m | 2.20, 1H, m |
| 20 | 2.32, 1H, m | 2.33, 1H, m |
| | 2.68, 1H, m | 2.68, 1H, m |
| 22 | 0.91, 3H, d(7.5) | 0.92, 3H, d(7.5) |
| 23 | 1.84, 3H, br.s | 1.89, 3H, d(1.0) |
| 24 | 1.10, 3H, d(6.5) | 1.09, 3H, d(6.5) |
| 25 | 2.32, 1H, m | 2.33, 1H, m |
| | 2.68, 1H, m | 2.68, 1H, m |
| 8-OCH₃ | 3.31, 3H, s | 3.31, 3H, s |
| 2′ | 3.70, 1H, dd(9.0, 4.0) | 3.74, 1H, m |
| 3′ | 2.96, 1H, dd(14.5, 9.0) | 2.95*, 1H, m |
| | 3.22, 1H, dd(14.5, 4.0) | 3.34^{b}, 1H, m |

^{a}Signals were assigned with aid of ¹H-¹H COSY, HMQC, and gHMBC experiments.
*Overlapped signals.
^{b}Overlapped with solvent peak.

Table 15 ¹H NMR (500 MHz) spectral data of compounds 105 (NK30424A) and 106 (NK30424B) in D₂O.

TABLE 15

| Position | NK30424A^{a} | 105^{b} | NK30424B^{a} | 106^{b} |
|---|---|---|---|---|
| 2 | 2.67, 1H, dd(17.4, 11.8) | 2.67, 1H, dd(17.5, 12.0) | 2.52, 1H, dd(15.4, 10.2) | 2.52, 1H, dd(15.0, 10.5) |
| | 2.85, 1H, dd(17.4, 3.2) | 2.85, 1H, dd(17.5, 3.0) | 3.00, 1H, dd(15.4, 2.8) | 3.00, 1H, dd(15.0, 2.5) |
| 3 | 3.34, 1H, m | 3.35, 1H, m | 3.20, 1H, m | 3.20, 1H, m |
| 4 | 1.87, 1H, m | 1.84, 1H, m | 1.58, 1H, m | 1.58, 1H, m |
| | 2.11, 1H, m | 2.09, 1H, m | 1.87, 1H, m | 1.88, 1H, m |
| 5 | 2.15, 1H, m | 2.15, 2H, m | 2.21, 1H, m | 2.23, 2H, m |
| | 2.13, 1H, m | | 2.27, 1H, m | |
| 6 | 5.81, 1H, ddd(16.0, 6.4, 6.4) | 5.82, 1H, dt(16.0, 6.5) | 5.86, 1H, ddd(16.2, 7.4, 7.4) | 5.86, 1H, dt(16.0, 7.0) |
| 7 | 5.48, 1H, dd(16.0, 7.4) | 5.48, 1H, dd(16.0, 7.0) | 5.49, 1H, dd(16.2, 6.8) | 5.48, 1H, dd(16.0, 7.0) |
| 8 | 3.55, 1H, dd(9.4, 7.4) | 3.55, 1H, dd(9.0, 7.0) | 3.58, 1H, dd(9.4, 6.8) | 3.59, 1H, dd(9.0, 6.5) |
| 9 | 3.75, 1H, dd(9.4, 4.8) | 3.75, 1H, dd(9.0, 6.0) | 3.75, 1H, dd(9.4, 2.4) | 3.75, 1H, dd(9.0, 3.0) |
| 10 | 2.03, 1H, m | 2.03, 1H, m | 2.05, 1H, m | 2.04, 1H, m |
| 11 | 4.94, 1H, d(2.4) | 4.94, 1H, d(2.5) | 5.09, 1H, br.d(2.4) | 5.08, 1H, d(2.0) |
| 13 | 5.26, 1H, br.d(9.6) | 5.27, 1H, d(11.0) | 5.30, 1H, br.d(9.6) | 5.30, 1H, br.d(10.5) |
| 14 | 3.63, 1H, dq(9.8, 6.4) | 3.62, 1H, dq(10.0, 6.5) | 3.64, 1H, dq(9.6, 6.6) | 3.65, 1H, dq(10.0, 6.5) |
| 16 | 2.72, 2H, m | 2.74, 2H, m | 2.75, 2H, m | 2.74, 2H, m |
| 17 | 4.19, 1H, m | 4.19, 1H, m | 4.20, 1H, m | 4.20, 1H, m |
| 18 | 1.50, 1H, m | 1.51, 1H, m | 1.51, 1H, m | 1.51, 1H, m |
| | 1.60, 1H, m | 1.60, 1H, m | 1.61, 1H, m | 1.62, 1H, m |
| 19 | 2.41, 1H, m | 2.42, 1H, m | 2.45, 1H, m | 2.43, 1H, m |
| 20 | 2.46, 1H, m | 2.46, 1H, m | 2.48, 1H, m | 2.50, 1H, m |
| | 2.73, 1H, m | 2.72, 1H, m | 2.77, 1H, m | 2.78, 1H, m |
| 22 | 0.98, 3H, d(7.2) | 0.99, 3H, d(7.0) | 1.00, 3H, d(7.4) | 1.00, 3H, d(7.0) |
| 23 | 1.78, 3H, d(1.0) | 1.79, 3H, br.s | 1.83, 3H, d(1.2) | 1.82, 3H, br.s |
| 24 | 1.15, 3H, d(6.6) | 1.15, 3H, d(6.5) | 1.16, 3H, d(6.6) | 1.16, 3H, d(7.0) |
| 25 | 2.45, 1H, m | 2.45, 1H, m | 2.47, 1H, m | 2.48, 1H, m |
| | 2.80, 1H, m | 2.78, 1H, m | 2.80, 1H, m | 2.80, 1H, m |
| 8-OCH₃ | 3.31, 3H, s | 3.32, 3H, s | 3.35, 3H, s | 3.35, 3H, s |
| Cys-2′ | 3.96, 1H, dd(9.6, 4.4) | 3.97, 1H, dd(7.5, 4.0) | 3.95, 1H, dd(7.8, 4.0) | 3.95, 1H, dd(8.0, 4.0) |
| Cys-3′ | 3.05, 1H, dd(14.6, 9.6) | 3.06, 1H, dd(14.5, 8.0) | 3.10, 1H, dd(14.2, 7.8) | 3.10, 1H, dd(14.5, 8.0) |
| | 3.21, 1H, dd(14.6, 4.4) | 3.22, 1H, dd(14.5, 4.5) | 3.25, 1H, dd(14.2, 4.0) | 3.24, 1H, dd(14.5, 4.0) |

^{a}NMR spectral data of NK30424A and NK30424B listed here were taken from literature (J. Antibiot. 2001, 54, 1111-1115.).
^{b}Signals were assigned with aid of ¹H-¹H COSY, HMQC, and gHMBC experiments in this study.

Table 16 ¹³C NMR (125 MHz) spectral data of compounds 105 (NK30424A) and 106 (NK30424B) in D₂O, and 144 and 145 in MeOH-d₄.

TABLE 16

| Position | NK30424A^{a} | 105^{b} | NK30424B^{a} | 106^{b} | 144^{b} | 145^{b} |
|---|---|---|---|---|---|---|
| 1 | 173.2 | 172.5 | 172.9 | 172.7 | 171.7 | 171.2 |
| 2 | 39.4 | 38.9 | 41.9 | 41.4 | 39.5 | 43.1 |
| 3 | 41.6 | 41.0 | 40.5 | 40.0 | 41.3 | 41.8 |
| 4 | 31.9 | 31.2 | 34.1 | 33.6 | 31.7 | 34.8 |
| 5 | 29.1 | 28.6 | 28.7 | 28.2 | 29.0 | 29.4 |
| 6 | 137.9 | 137.4 | 135.7 | 135.3* | 137.5 | 135.4 |
| 7 | 128.4 | 127.9 | 131.1 | 130.6 | 130.4 | 133.4 |
| 8 | 83.2 | 82.7 | 82.9 | 82.4 | 84.6 | 84.0 |
| 9 | 73.2 | 72.8 | 73.4 | 72.8 | 73.1 | 73.1 |
| 10 | 39.0 | 38.5 | 39.1 | 38.6 | 39.4 | 39.8 |
| 11 | 80.3 | 79.8 | 80.9 | 80.5 | 82.0 | 81.4 |
| 12 | 135.0 | 135.3 | 135.4 | 135.3* | 135.5 | 135.8 |
| 13 | 126.6 | 126.4 | 127.1 | 126.5 | 128.6 | 129.1 |
| 14 | 46.7 | 46.3 | 46.8 | 46.2 | 47.1 | 47.1 |
| 15 | 216.2 | 215.7 | 216.0 | 215.8 | 213.5 | 213.6 |
| 16 | 48.6 | 48.1 | 48.6 | 48.1 | 41.3 | 41.3 |
| 17 | 65.4 | 64.9 | 65.3 | 64.3 | 21.5 | 21.5 |
| 18 | 41.5 | 41.1 | 41.5 | 41.0 | 35.3 | 35.3 |
| 19 | 27.3 | 26.9 | 27.4 | 26.9 | 31.4 | 31.4 |
| 20 | 38.1 | 37.6 | 38.1 | 37.6 | 38.6 | 38.7 |
| 21 | 177.5 | 177.1* | 177.5 | 176.9 | 175.8 | 175.8 |
| 22 | 11.6 | 11.1 | 11.3 | 10.8 | 11.8 | 11.6 |
| 23 | 14.5 | 14.0 | 14.4 | 13.9 | 13.9 | 14.4 |
| 24 | 16.0 | 15.5 | 15.9 | 15.4 | 16.4 | 16.3 |
| 25 | 37.1 | 36.5 | 37.0 | 36.6* | 38.6 | 38.7 |
| 26 | 177.4 | 177.0* | 177.4 | 176.9* | 175.8* | 175.8* |
| 8-OCH3 | 56.6 | 56.1* | 57.0 | 56.5* | 57.1* | 57.1* |
| 1′ | 177.4 | 177.1 | 177.4 | 177.0* | 175.8* | 175.8* |
| 2′ | 56.6 | 56.1* | 57.0 | 56.5* | 56.8* | 56.8* |
| 3′ | 37.1 | 36.6 | 37.0 | 36.6* | 32.8 | 33.2 |

^{a}NMR spectral data of NK30424A and NK30424B listed here were taken from literature (J. Antibiot. 2001, 54, 1111-1115).
^{b}Signals were assigned with aid of ¹H—¹H COSY, HMQC, and gHMBC experiments.
*Overlapped signals or signals that could be interchangeable.

### EXAMPLE III

Fermentation Optimization and Natural Products Isolation from S. platensis.

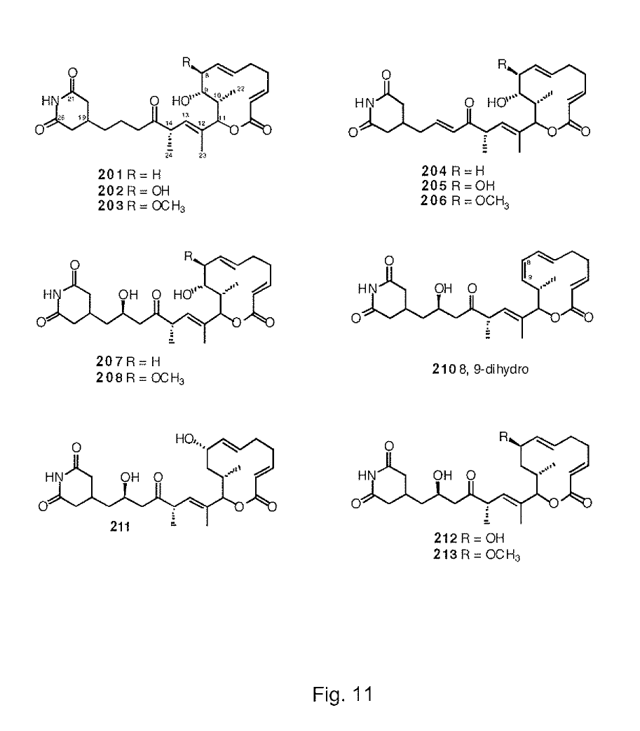

It has been well known that the culture conditions (e.g. medium composition, aeration, culture vessel, temperature, PH, etc.) can affect the secondary metabolite profiles of the microorganism. Bode, H. B., B. Bethe, R. Hofs, and A. Zeeck. Big effects from small changes: possible ways to explore nature's chemical diversity. Chembiochem. 2002, 3, 619-627. The relative abundance of each component in the mixture of metabolites obtained from S. platensis cultures was found to be variable and dependent upon the culture conditions used. Metabolite titers were optimized by altering the seed culture, amount of adsorbent resin supplemented, duration of fermentation time, and level of oxygen supplied. High yields were obtained by the combination of (a) relatively young seed culture (30 hrs), (b) production medium supplemented with high percentage of resins, (c) relatively short fermentation time, and (d) reduced oxygen supply. Thus, no adverse effect on S. platensis growth was observed upon supplementation to the production medium with 2%-15% (w/v) XAD-16 or 2%-7% HP-20 resins. In summary, the highest yields for production of compounds 201-208 were obtained when 50 mL production medium in a 250-mL flask (or 500 mL production medium in a 2-L flask) supplemented with 15% XAD-16 resin was cultured for 42-48 hours. Under the optimized condition, compounds 201-208 were produced and purified from wild-type S. platensis, with isolated yields of 2-35 mg/L (Table 17, FIG. 11). Ju, J.; Lim, S.-K.; Jiang, H.; Seo, J.-W; Shen, B. J. Am. Chem. Soc., 2005, 127, 11930-11931.

TABLE 17
A library of compounds generated by fermentation methods and
semi-synthesis.

| | | | | | | | C2/C3 double | |
|---|---|---|---|---|---|---|---|---|
| | | H₂O mediated | | | | Cysteine | reduction | N-acylated |
| | Origin | rearrangement products | | | | addults | products | products |
| 201 | S. platensis | 201a | 202b | 201c | 201d | 201e | | |
| | MgsJ mutant | | | | | | | |
| 202 | S. platensis | 202a | 203b | 202c | 202d | 202e | | |
| | MgsJ mutant | | | | | | | |
| 203 | S. platensis | 203a | 203b | 203c | 203d | 203e | 203f | 203g |
| 204 | S. platensis | 204a | 204b | 204c | 204d | | | |
| | MgsJ mutant | | | | | | | |
| 205 | S. platensis | 205a | 205c | 205c | 205d | | | |
| | MgsJ mutant | | | | | | | |
| 206 | S. platensis | 206a | 206b | 206c | 206d | | | |
| 207 | S. platensis | 207a | 207b | 207c | 207d | 207e | | |
| | MgsJ mutant | | | | | | | |
| | S. amphibiosporus | | | | | | | |
| 208 | S. platensis | 208a | 208b | 208c | 208d | 208e | | |
| 209 | S. amphibiosporus | | | | | | 209f | |
| 210 | S. amphibiosporus | 210a | 210b | 211c | 210d | 210e | 210f | |
| | LtmK mutant | | | | | | | |
| 211 | S. amphibiosporus | 211a | 211b | 211c | 211d | 211e | | |
| 212 | S. amphibiosporus | 212a | 212b | 212c | 212d | 212e | | |
| | containing MgsK | | | | | | | |
| | gene mutant strain | | | | | | | |
| 213 | S. amphibiosporus | 213a | 213b | 213c | 213d | 213e | | |
| | containing MgsI, J, K | | | | | | | |
| | genes mutant strain | | | | | | | |

Fermentation and Natural Product Isolation from S. amphibiosporus

The metabolite profiles of S. amphibiosporus were also examined at various conditions by altering the media composition and particularly by supplementation of XAD-16 adsorbent resin. We have found that the titer of LTM (209) was greatly enhanced up to 20-fold when 2-3.5% (w/v) XAD-adsorbent resin was supplemented in the production medium. In addition, under the optimized condition, three minor metabolites (207, 210, 211) were observed. Unlike that up to 15% XAD-16 resin could be added in the culture medium of S. platensis, high percentage of resin (>4%) in culture medium of S. amphibiosporus led to sharply reduced or no production of LTM. Under the optimized condition, compounds 209, 210, 207, and 211 were purified from wild-type S. amphibiosporus, in an isolated yield of approximate 70 mg/L, 2 mg/L, 5 mg/L and 5 mg/L, respectively (Table 17, FIG. 11).

Engineering and Isolation of Novel iso-MGS and LTM Analogs by Combinatorial Biosynthesis Methods

Gene inactivation in S. platensis. The biosynthetic gene cluster for isomigrastatin (203) has been cloned and sequenced in our lab. The iso-MGS gene cluster consisted of three putative post-tailoring enzymes, namely, MgsI, MgsJ, and MgsK. MgsJ shows 53% identity and 68% similarity to MitM (NP617440), an O-methyl transferase, and highly conserved SAM binding motif. The three tailoring genes mgsI, J, andKwere mutated according to the established protocol based on λ-RED PCR targeting system. No new metabolites were observed from both the mgsI and mgsK mutants. While the mgsJ mutant, predominantly accumulated compounds 201 and 202. When fermented using the same condition as that for wild-type strain, mgsJ mutant showed similar metabolite profiles (201, 202, 204, 205, 207) as observed from wild-type strain except that the three methylated compounds (203, 206, 208) were not produced.

Gene inactivation in S. amphibiosporus. The LTM (209) biosynthetic gene cluster has been recently identified in our lab. LtmK consisting of 413 amino acids (1239 bps) was found to be a putative post-PKS tailoring enzyme in gene cluster for biosynthesis of LTM. Homology alignment of the deduced amino acids sequence revealed that LtmK is a putative cytochrome P-450 protein showing 52% similarity to PikC hydroxylase involved in prikromycin biosynthesis in S. venezyelae. To explore the exact role of the ItmK gene product in LTM biosynthesis, ItmK was inactivated by replacement with apramycin resistance gene (Apr^{R}) cassette using REDIRECT technology method. Fermentation of the ΔItmK mutant strain was carried out similarly as conducted for WT-strain. The LtmK mutant exclusively produced 8,9-dihydro-LTM (210), the isolated yield of which is 15 mg/L.

Introduction of the tailoring steps of iso-MGS into LTM. The three tailoring genes mgsI, mgsJ, and mgsK from S. platensis, the iso-MGS biosynthetic pathway, were introduced into the S. amphibiosporus wild-type strain, the LTM producer, resulted in a series of mutants. Among the mutants generated, the mgsK-S. amphibiosporus mutant and mgsIJK-S. amphibiosporus mutant were found to produce new compounds 212 and 213, respectively. The isolated yields of 212 and 213 were each 15 mg/L. [12, 9]

H₂O-Mediated Ring-Expansion and Ring-Opening Rearrangement

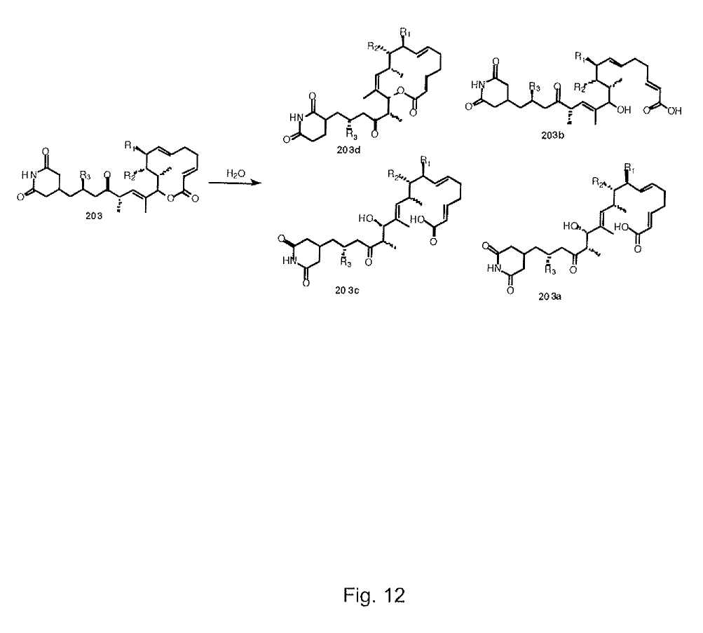

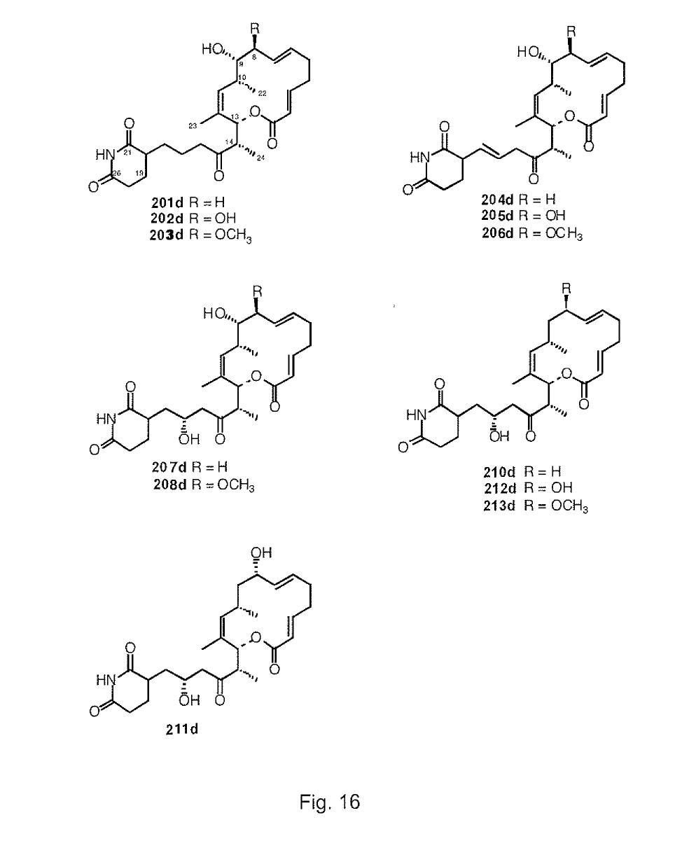

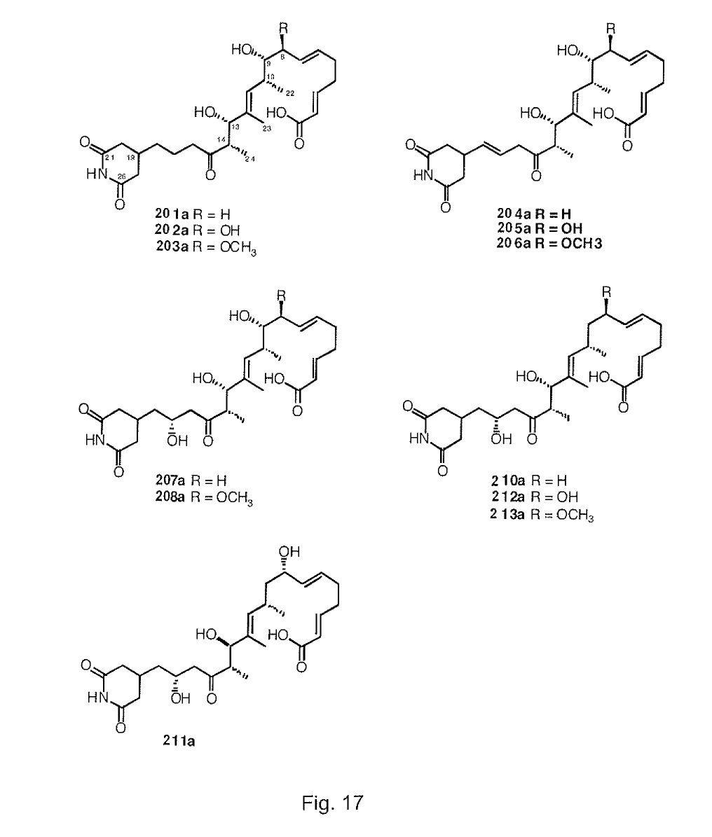

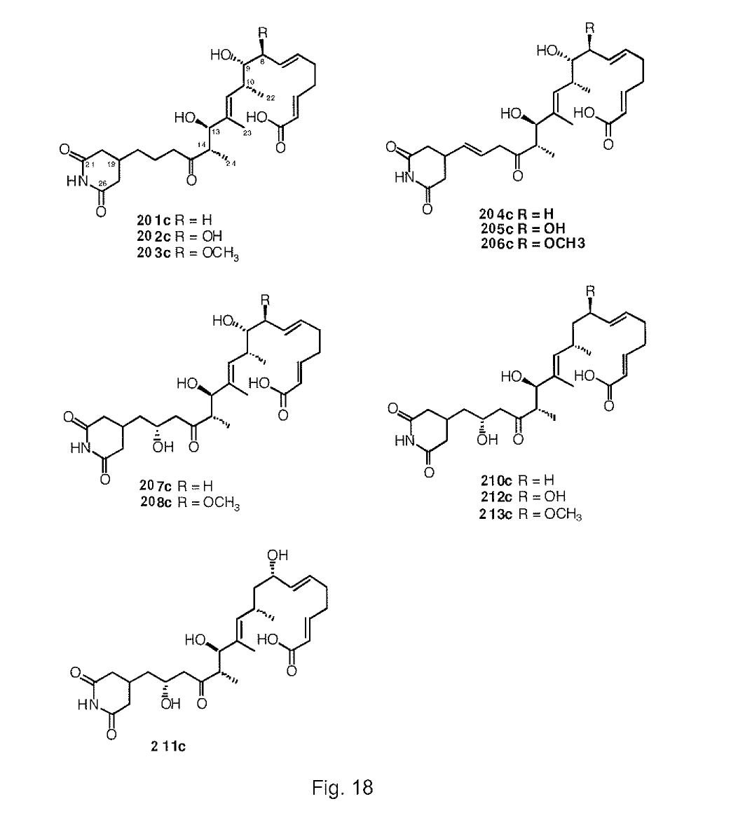

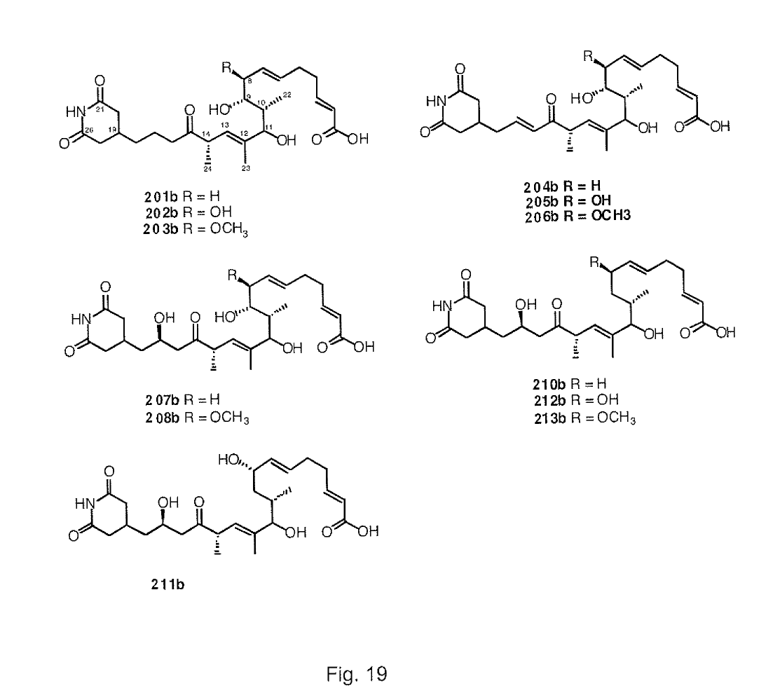

We have reported that isomigrastatin (203) suffered from H₂O-mediated rearrangement into their corresponding 14-membered migrastatin (203d) and open-chain form of dorrigocins (203a-203c) products, two families of bioactive molecules previously discovered (FIG. 12). Ju, J.; Lim, S.-K.; Jiang, H.; Shen, B. J. Am. Chem. Soc. 2005, 127, 1622-1623. The other congeners (201-202, 203-208, 210-213) but LTM (209) undergo the similar H₂O-mediated ring-opening and ring-expansion rearrangements to their corresponding MGS and DGN analogs (Table 17, FIG. 16-19).

Chemical Modification

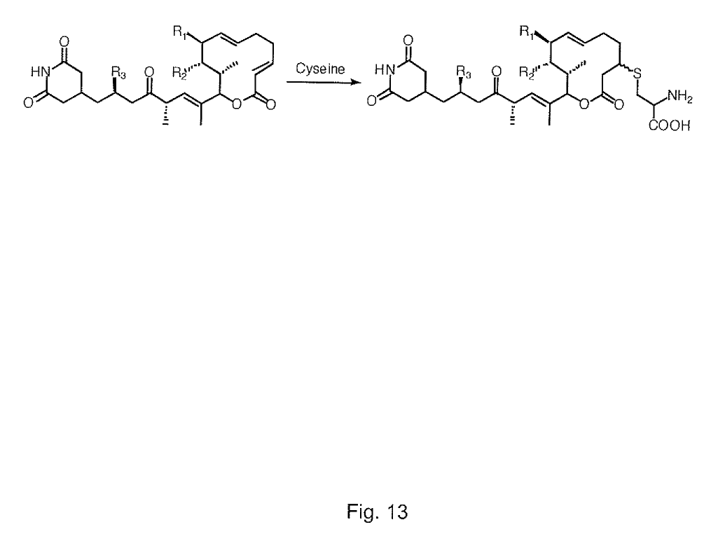

1,4-Michael addition by a sulfer nucleophile (i.e., cysteine) to access the NK30424 scaffold. The C2/C3 double of all the 12-membered macrolactones (201-208, 210-213) readily suffered from 1,4-Michael addition reaction, and subsequently each macrolide resulted in a pair of diasteroisomers (FIG. 13). LTM (209) was not examined, while migrastatin does not possess this property.

These results provide experimental evidence supporting the idea that the C8-C9 double bond changes the 12-membered ring configuration, thereby the propensity to undergo the ring-opening and ring-expansion rearrangement.

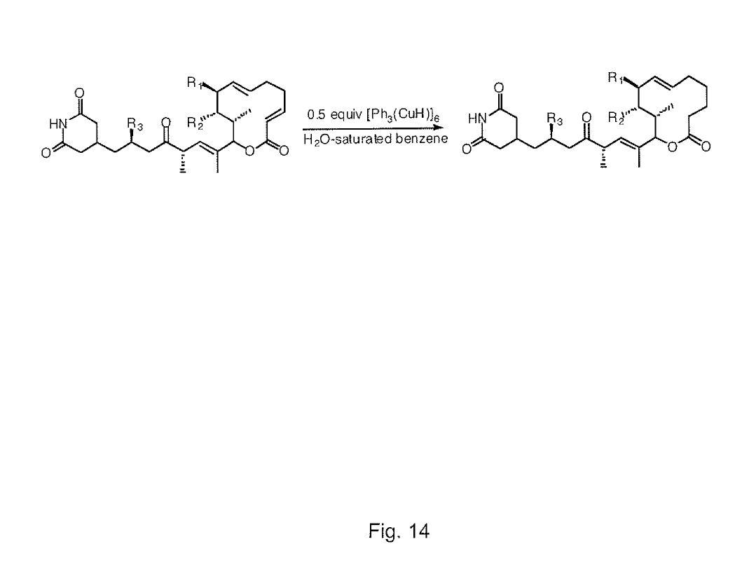

Regioselective 1,4-reduction to access the reduced NK30424 scaffold. The α,β-unsaturated ester group could be regioselectively reduced by Stryker's reagent. Angelo de Fatima, Synlett., 2005,11,1805-1806. In order to saturate the C2/C3 double bond to stabilize the 12-membered macrolides, compounds 203, 207, 209, and 210 were reduced with this reagent (FIG. 14). The reactions for 203, 209, and 210 were complete in 2, 8, and 12 hrs, respectively, as monitored by HPLC (yields>80%). Reduction of 207 failed under the similar condition. The reduced compounds 203f, 209f, 21 Of can survive in H₂O for more than 24 hrs.

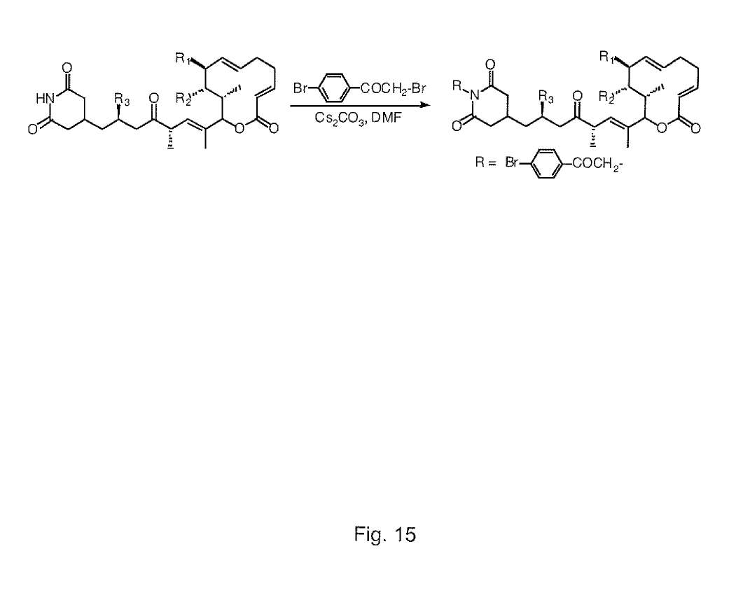

N-acylation. The glutarimide moiety in the macrolides (both 12-membered and 14-membered) provided an excellent opportunity for modification via N-acylation. Treatment of iso-MGS (203) with p-bromophenacyl bromide and Cs₂CO₃ in DMF led to N-p-bromophenacylated-isomigrastatin (FIG. 15).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A glutarimide-containing polyketide analog selected from the group consisting of: 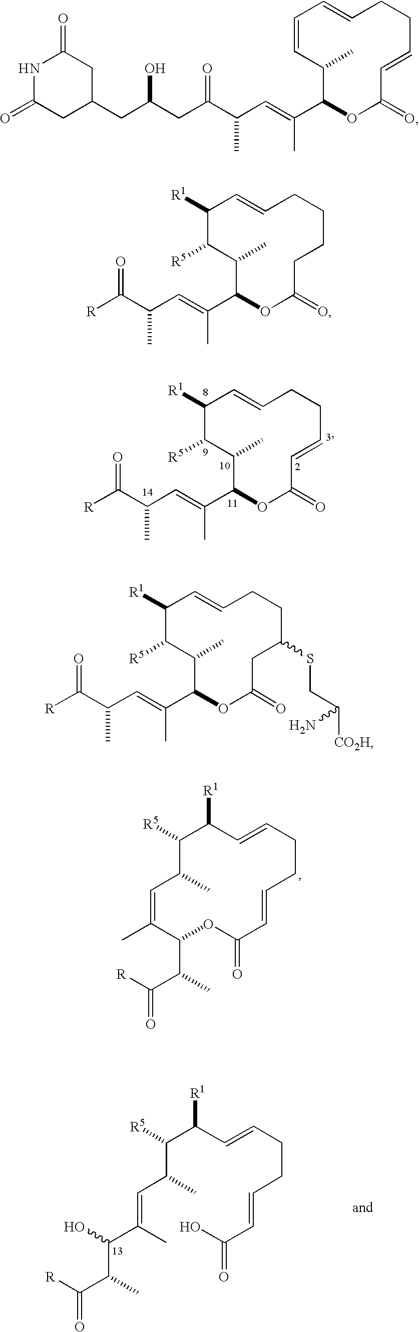 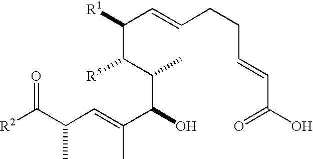
wherein R is selected from the group consisting of: 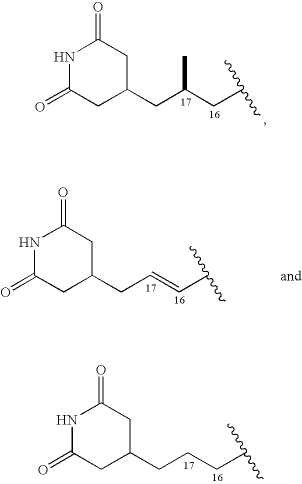
wherein R¹ and R⁵ are independently selected from the group consisting of H, OH, OCH₃, with the provisio that in compound 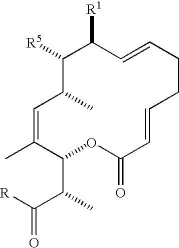
R is not 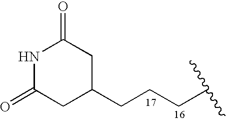
when R¹ is OCH₃ and R⁵ is OH.

2. A method of synthesizing a glutarimide-containing polyketide analog said method comprising:
(I) fermenting
(a) a wild type strain of Streptomyces amphibiosporus or Streptomyces platensis in a culture medium under conditions whereby said wild type strain expresses said glutarimide-containing polyketide analog; or
(b) a mutant strain of Streptomyces amphibiosporus or Streptomyces platensis in a culture medium under conditions whereby said mutant strain expresses said glutarimide-containing polyketide analog; wherein the mutant strain of Streptomyces amphibiosporus or Streptomyces platensis comprises a nucleic acid selected from the group consisting of SEQ ID. 1, SEQ ID. 2, SEQ ID. 3, SEQ ID. 4, and combinations thereof; or
(c) a recombinantly modified iso-migrastatin or lactimidomycin gene cluster under conditions whereby said gene cluster expresses said glutarimide-containing polyketide analog, wherein said gene cluster is present in a bacterium selected from the group consisting Streptomyces amphibiosporus and Streptomyces platensis; and
(II) isolating at least one glutarimide-containing polyketide analog.

3. A method of chemically modifying a glutarimide-containing polyketide analog said method comprising:
(I) obtaining an isolated glutarimide-containing polyketide analog, according to claims 2;
(II) conducting at least one of the modification to said glutarimide-containing polyketide analog to result in a chemically modified glutarimide-containing polyketide analog, wherein the modification is selected from the group consisting of:
(a) a water mediated ring opening of the glutarimide-containing polyketide analog;
(b) a 1,4-addition of the glutarimide-containing polyketide analog by a sulfur-containing nucleophile;
(c) a regioselective 1,4-reduction of the glutarimide-containing polyketide analog; and
(d) a N-acylation of the glutarimide moiety of the glutarimide-containing polyketide analog; and
(III) isolating and purifying at least one chemically modified glutarimide-containing polyketide analog.

4. A glutarimide-containing polyketide analog produced by the method of claims 2 or 3.

5. A pharmaceutical composition comprising
(a) An analog according to claim 1 or claim 6; or
(b) a pharmaceutically-acceptable salt of said analog; and
(c) a pharmaceutically-acceptable carrier.

6. An isolated, purified or enriched nucleic acid comprising a sequence selected from the group consisting of SEQ ID. Nos. 1, 2, 3 and 4; the sequences complimentary to SEQ ID. Nos. 1, 2, 3 and 4 and fragments comprising at least 10 consecutive nucleotides of the sequences complementary to SEQ ID. Nos. 1, 2, 3 and 4.

7. An isolated, purified or enriched nucleic acid capable of hybridizing to the nucleic acid of claim 6 under conditions of high stringency.


SEQUENCE LISTING (4 sequences)

SEQ ID NO: 1 - DNA, 1242 bp, Streptomyces amphibiosporus
gtgaccacccacctcaccaccgacatcgacgagatcgtcacggacgtcttccagtccaccgaggggaagaagaacccctaccccctgtaccggcgcctccaggagctgggccaggtgcaccgctccgagcaactgggctgggtcgccacgggatacgaggtgtgcagcgccgcgctgcgcgacccccgcgtcatcaagggccccgagcagatccagccgggccgccccgaccccgccgagcactccgccgaggcgttgctgcgcggcacgatgcaccggctcgacccgccggaccacacccggctgcgccgcctcgtcaacggcgccttcacgccgcgcagcgtcgccgcgctggagccggacatccaggaactgatcgacgacctgatcacaccggccgtcaagaaggcggaggcgggcgagcccgtcgacatgatgagcggcttcgccttcccgctctccgtcgccgtgatcggccgcatgctcggcgtgcccgcgtccgactggcaccgcttccacgacgtcgtgctcgacctgtcctccatggtcgaactcggcttcaccggcgacgagttgcccaaggccgacgccgccgcggacgaactgatcgcctacttccgcaagttgggcgccgagcgcatgcgcaaccccgccgacgacctgacctccacgctcgccaacgcgaccgaggccggtgaccgcctcaccgagcaggaactcgtcaccatgctgatcctgctgttcatggccggtttcgagacgacgacccactccatgggcaacggcatgttcgccctgctggagaacccggagcagacgcagtggctgcgccgcaacatggacgccatgcccgcggccgtcgaggagctgatccgctacgactccccggtgcagttcatcgccggctacaccaaggagcccgtggaactggcggacggcaccgccgtccccgcggacgagtatctgttcctgatgatcggcgcggccaaccgcgacccgcgcgtcttctccgaccccgaactgctgcgtctggaccgcggtgaggccgcgccgatgagcttcggcggcggcatccactactgcctcggcgcgggcctggcccggctggagatccggaagatcttcacctcgctgctcacccgcttctccgcgatcgaactggccgagcccgaaccggaacgccgcagcggactcgccctgcgcggctacgcccgcatcccgatgtggctcaccccggcgtaa

SEQ ID NO: 2 - DNA, 1212 bp, Streptomyces platensis
gtgaccggaccgaccgacggcggcatcccgcctgccttccccttcccccgcacctgcccctaccggctgcccgccgagtacaacggcctccgcgagagcgggcccgtgtcgaaggtgatcctgccgaccggtaagccggcgtggatcctgacccgctacgacgacgttcgcaaggcgctgctcgacccgcatctcagctccgaccgggccgatccgaactttccgatgctggtcgagctcgcgcggagcgacaaggactccacactgtccctggcgggcatggacgcgcccgaacacacccgcgcgcggcgcgccgtggtcggcgagttcaccttccgccggatggaagccctgcgcccgcgcgtccaggagatcgtcgacgagtgcgtcgacgcgatgctcgccggccccaacccggccgacctggtgaagacgatgtccttcccggtgccctccctggtcatctgcgagctgctcggcgtgtccgtcgccgaccgcgacttcttcgaggaccgcacctcccgcatgctcagcatgaccctcccgccgctgacccggcagcaggcgttcttcgacctgcagacctacttggacgagctcgtgacggccaaggagaaagtcccgggggacgacctgctcagccgccagatcgccaagggccgaaaggacagcgcgtacgaccacgacgcactcgtcgacctggcgttcctgctgctgaccgccgggcacgacacgaccggaaacatgatctcgctgggcatgctcgccctgatggagacgcccgagctgcgggcgcgcatcaccgacgaccccggcaccacgccgcaggtcgtcgaggaactgctgcgctacttcaccatcaccgacatcatcaccacgcgcgtggccaaggaggacgtcgagatcggcgggcagaccatccgcgccggcgaaggcgtcttcgcgctgggcggctcggccaaccacgacccggacgtcttcgagaaccccggaaagctggacgtcgaccgcggcgcacgccagcacctcgccttcggccacggaccgcaccagtgcctcgggcagagcctcgcccgcatggaactggagatcgtctacgacaccctgctccgccgcatccccggactccggccggccggccccgctgaagacctgccgctgaagaacgacgcggccatcttcggcctgcacgaactcccggtcatctggtag

SEQ ID NO: 3 - DNA, 846 bp, Streptomyces platensis
atgtccagcacgtccacgaccgcccccgtctccgtccccgtctccgcccccgtccccgaagaggtcggacacctctacgaccgcctcaccgcactggacaccgaagcggccggcggcagcctccacctcggctactgggacgtcgacgacaacgacaccccgctcgtggaagcggccgaccggctcaccgacacgatgaccgaccgcctgcggatcgaccagggacagcgggtcctcgacgtcggctgcggagtcggccagccggccatgcggatcgcccggcgcaccggcgcccatgtcacgggcatcgcgatcagcaaggaccagatcgcccgcgccaccgccctcgccgagggcgccggcctgagcgaccgcgtggagttccggcacgccgacgccatggaactgcccttccccgacgactccttcgacgccgccatcgccatcgagtcgatcttccacatgcccgaccgcggacgggtcctcgccgagatccgccgcgtactgcgccccggcggccgcctggtcctcaccgacttcttcgagcgcggccccgtccccgccgagaagcagcccgcggtggaccggctcctccgcgacttcatcatgacgctggcccggcccgaggactacgtgcccatgctgcgcgacgcaggcctgcgcttcgtcgagctcctcgacatcaccgagcagagcgtgcgtcagaccttcgagcagatgagccagggctcccaggagatgcagaccgtcttcgacgacgaggcagaggaaaagttcagccccgcctccatgatcgacgtcgacgaattcggctccgttctgctgaccgcccaaaagcccctctga

SEQ ID NO: 4 - DNA, 1017 bp, Streptomyces platensis
atgaccgttccgtccacgggcaccgaagtccggctcgccacccgccccgaggggtggccgaccactgagaacttctcggtcgtgcaggcggaaccgcccgcggtcaggaccggccaggtgctgatccgcaacctggtgatgagcgtcgacccgtacatgcgcgggcggatgaacaaaaccaggtcctacgttccgccgttcgccgtcggcaaggcgctcgacgggggcgccgtcggcgaggtcgtcgtctcgaagtcatcgcaactcgccgtcggtgacctggtcctgcacggcctcggctggcgggagtacgccgtcgtgggcgctgccggcgcggtcaggatcgacccggcgctcgcgccgcccggcgcgtatctcggagtgctcggcatgccggggcacgccgcctacacggggttgctcaaggccgccgaattccggcccggcgacaccgtgttcgtctccggggccgcgggcgcggtgggctccctcgtcggtcagatcgcccggctctgcggcgcggaacgcgtgatcggatcggcgggcagcgccgagaaagtcgcctatctgaccggggagctcggcttcgacgcggcattcgactacaaggacgggccggttctcgaacagctggcgaaggccgcgccgacgggcatcgacgtgtacttcgacaacgtgggcggcgaccacctggacgccgccctggtcctggccaggatgggcgcgcggttcgccctctgcggcaacatctcgcaggccaacgagaaggacccgccggccggcccacggaacctgacgcaggccatcgccaagggcatcaccctgcgcggcgtcctcgtcggaggccacgccgacctcccggacgagttcaccgcccgcatgggtggctggctggcagacgggagaatctcctaccgggagaccgtcgtcaggggactggagaacgcacccgccgccttcatcgacatgctgcgcggcgccaacaccggcaaaatgctcgtgagaatcgccgaatga